SEQUENCE LISTING (2 sequences)

SEQ ID NO: 1 - protein, 306 aa, Homo sapiens [sequence omitted]

SEQ ID NO: 2 - protein, 306 aa, Homo sapiens [sequence omitted]

(19) United States
(12) Patent Application Publication
(10) Pub. No.: US 2025/0304559 A1
(43) Pub. Date: Oct. 2, 2025

(54) Ether-Linked Antiviral Compounds

(71) Applicant: Pfizer Inc., New York, NY (US)

(72) Inventors: Dafydd Rhys Owen, Concord, MA (US); Matthew Richard Reese, Mystic, CT (US); Matthew Forrest Sammons, Quincy, MA (US); Jamison Bryce Tuttle, Marblehead, MA (US); Qingyi Yang, Lexington, MA (US)

(73) Assignee: Pfizer Inc., New York, NY (US)

(21) Appl. No.: 18/552,453

(22) PCT Filed: Mar. 25, 2022
(86) PCT No.: PCT/IB2022/052755

§ 371 Date: Sep. 26, 2023

Related U.S. Application Data
(60) Provisional application No. 63/167,714, filed on Mar. 30, 2021.

(51) Int. Cl. C07D 403/12; A61K 31/4025; A61K 31/422; A61K 31/427; A61K 31/454; C07D 401/14; C07D 403/14; C07D 413/14; C07D 417/14

(52) U.S. Cl. CPC C07D 403/12; A61K 31/4025; A61K 31/422; A61K 31/427; A61K 31/454; C07D 401/14; C07D 403/14; C07D 413/14; C07D 417/14

(57) ABSTRACT

The invention relates to compounds of Formula I

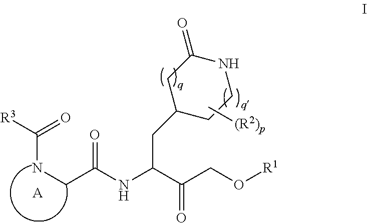

and pharmaceutically acceptable salts thereof wherein R¹, R², R³, p, q, q′ and Ring A are as defined herein, pharmaceutical compositions comprising the compounds, methods of treating coronavirus infection such as COVID-19 in a patient by administering therapeutically effective amounts of the compounds, and methods of inhibiting or preventing replication of coronaviruses such as SARS-CoV-2 with the compounds.

### BACKGROUND OF THE INVENTION

The invention relates to compounds and methods of inhibiting viral replication activity comprising contacting a SARS-CoV-2-related 3C-like (“3CL”) proteinase with a therapeutically effective amount of a SARS-CoV-2-related 3C-like protease inhibitor. The invention also relates to methods of treating Coronavirus Disease 2019 (“COVID-19”) in a patient by administering a therapeutically effective amount of a SARS-CoV-2-related 3C-like protease inhibitor to a patient in need thereof. The invention further relates to methods of treating COVID-19 in a patient, the method comprising administering a pharmaceutical composition comprising a therapeutically effective amount of the SARS-CoV-2-related 3C-like protease inhibitor to a patient in need thereof.

A worldwide outbreak of Coronavirus Disease 2019 (“COVID-19”) has been associated with exposures originating in late 2019 in Wuhan, Hubei Province, China. By mid-2020 the outbreak of COVID-19 had evolved into a global pandemic with millions of people having been confirmed as infected and resulting in hundreds of thousands of deaths and by March 2021 there have been approximately 125 million confirmed cases and approximately 2.75 million deaths. The causative agent for COVID-19 has been identified as a novel coronavirus which has been named Severe Acute Respiratory Syndrome Corona Virus 2 (“SARS-CoV-2”). The genome sequence of SARS-CoV-2 has been sequenced from isolates obtained from nine patients in Wuhan, China and has been found to be of the subgenus Sarbecovirus of the genus Betacoronavirus. Lu, R. et al. The Lancet, 395, 10224, 565-574; online Jan. 29, 2020. The sequence of SARS-CoV-2 was found to have 88% homology with two bat-derived SARS-like coronaviruses, bat-SL-CoVZC45 and bat-SL-CoVZXC21, which were collected in 2018 in Zhoushan, eastern China. SARS-CoV-2 was also found to share about 79% homology with Severe Acute Respiratory Syndrome Corona Virus (“SARS-CoV”), the causative agent of the SARS outbreak in 2002-2003, and about 50% homology with Middle East Respiratory Syndrome Coronavirus (“MERS-CoV”), the causative agent of a respiratory viral outbreak originating in the Middle East in 2012.

Based on a recent analysis of 103 sequenced genomes of SARS-CoV-2 it has been proposed that SARS-CoV-2 can be divided into two major types (L and S types) with the S type being ancestral and the L type having evolved from the S-type. Lu, J.; Cui, J. et al. On the origin and continuing evolution of SARS-CoV-2; http://doi.org/10.1093/nsr/nwaa036. The S and L types can be clearly defined by just two tightly linked SNPs at positions 8,782 (orf1ab:T8517C, synonymous) and 28,144 (ORF8: C251T, S84L). In the 103 genomes analyzed approximately 70% were of the L-type and approximately 30% were of the S-type. It is unclear if the evolution of the L-type from the S-type occurred in humans or through a zoonotic intermediate but it appears that the L-type is more aggressive than the S-type and human interference in attempting to contain the outbreak may have shifted the relative abundance of the L and S types soon after the SARS-CoV-2 outbreak began. The discovery of the proposed S- and L-subtypes of SARS-CoV-2 raises the possibility that an individual could potentially be infected sequentially with the individual subtypes or be infected with both subtypes at the same time. In view of this evolving threat there is an acute need in the art for an effective treatment for COVID-19 and for methods of inhibiting replication of the SARS-CoV-2 coronavirus.

Recent evidence clearly shows that the newly emerged coronavirus SARS-CoV-2, the causative agent of COVID-19 (Centers for Disease Control, CDC) has acquired the ability of human-to-human transmission leading to community spread of the virus. The sequence of the SARS-CoV-2 spike protein receptor-binding domain (“RBD”), including its receptor-binding motif (RBM) that directly contacts the angiotensin-converting enzyme 2 receptor, ACE2, is similar to the RBD and RBM of SARS-CoV, strongly suggesting that SARS-CoV-2 uses ACE2 as its receptor. Wan, Y.; Shang, J.; Graham, R.; Baric, R. S.; Li, F.; Receptor recognition by the novel coronavirus from Wuhan: An analysis based on decade-long structural studies of SARS coronavirus; J. Virol. 2020; doi:10.1128/JVI.00127-20. Several critical residues in SARS-CoV-2 RBM (particularly Gln⁴⁹³) provide favorable interactions with human ACE2, consistent with SARS-CoV-2's capacity for human cell infection. Several other critical residues in SARS-CoV-2's RBM (particularly Asn⁵⁰¹) are compatible with, but not ideal for, binding human ACE2, suggesting that SARS-CoV-2 uses ACE2 binding in some capacity for human-to-human transmission.

Coronavirus replication and transcription function is encoded by the so-called “replicase” gene (Ziebuhr, J., Snijder, E. J., and Gorbalenya, A. E.; Virus-encoded proteinases and proteolytic processing in the Nidovirales. J. Gen. Virol. 2000, 81, 853-879; and Fehr, A. R.; Perlman, S.; Coronaviruses: An Overview of Their Replication and Pathogenesis, Methods Mol. Biol. 2015; 1282: 1-23. doi:10.1007/978-1-4939-2438-7_1), which consists of two overlapping polyproteins that are extensively processed by viral proteases. The C-proximal region is processed at eleven conserved interdomain junctions by the coronavirus main or “3C-like” protease (Ziebuhr, Snijder, Gorbalenya, 2000 and Fehr, Perlman et al., 2015). The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. The SARS-CoV-2 3CL protease sequence (Accession No. YP_009725301.1) has been found to share 96.08% homology when compared with the SARS-CoV 3CL protease (Accession No. YP_009725301.1) Xu, J.; Zhao, S.; Teng, T.; Abdalla, A. E.; Zhu, W.; Xie, L.; Wang, Y.; Guo, X.; Systematic Comparison of Two Animal-to-Human Transmitted Human Coronaviruses: SARS-CoV-2 and SARS-CoV; Viruses 2020, 12, 244; doi:10.3390/v12020244. Very recently, Hilgenfeld and colleagues published a high-resolution X-ray structure of the SARS-CoV-2 coronavirus main protease (3CL) Zhang, L.; Lin, D.; Sun, X.; Rox, K.; Hilgenfeld, R.; X-ray Structure of Main Protease of the Novel Coronavirus SARS-CoV-2 Enables Design of α-Ketoamide Inhibitors; bioRxiv preprint doi: https://doi.org/10.1101/2020.02.17.952879. The structure indicates that there are differences when comparing the 3CL proteases of SARS-CoV-2 and SARS-CoV. In the SARS-CoV but not in the SARS-CoV-2 3CL protease dimer, there is a polar interaction between the two domains Ill involving a 2.60-Å hydrogen bond between the side-chain hydroxyl groups of residue Thr²⁸⁵ of each protomer, and supported by a hydrophobic contact between the side-chain of Ile²⁸⁶ and Thr²⁸⁵ Cγ₂. In the SARS-CoV-2 3CL, the threonine is replaced by alanine, and the isoleucine by leucine when compared with the same residues in the SARS-CoV 3CL. The Thr285Ala replacement observed in the SARS-CoV-2 3CL protease allows the two domains Ill to approach each other somewhat closer (the distance between the Cα atoms of residues 285 in molecules A and B is 6.77 Å in SARS-CoV 3CL protease and 5.21 Å in SARS-CoV-2 3CL protease and the distance between the centers of mass of the two domains Ill shrinks from 33.4 Å to 32.1 Å). In the active site of SARS-CoV-2 3CL, Cys¹⁴⁵ and His⁴¹ form a catalytic dyad, which when taken together with a with a buried water molecule that is hydrogen-bonded to His⁴¹ can be considered to constitute a catalytic triad of the SARS-CoV-2 3CL protease. In view of the ongoing SARS-CoV-2 spread that has caused the current worldwide COVID-19 outbreak, it is desirable to have new methods of inhibiting SARS-CoV-2 viral replication and of treating COVID-19 in patients.

### SUMMARY OF THE INVENTION

The present invention provides novel compounds which act in inhibiting or preventing coronavirus replication, such as SARS-CoV-2 viral replication, and thus are useful in the treatment of coronavirus infections including, but not limited to, COVID-19. The present invention also provides pharmaceutical compositions comprising the compounds and methods of treating coronavirus infections, including COVID-19 and inhibiting coronavirus replication, such as SARS-CoV-2 viral replication, by administering the compounds of the invention or pharmaceutical compositions comprising the compounds of the invention.

The following embodiments, including embodiments E1 to E32, are non-limiting embodiments of the present invention.

E1 is a compound of Formula I

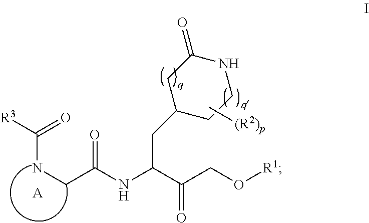

or a pharmaceutically acceptable salt thereof;
wherein
R¹ is selected from the group consisting of C₁-C₆ alkyl, C₃-C₆ cycloalkyl, (C₃-C₆ cycloalkyl)-C₁-C₆ alkyl, C₆-C₁₀ aryl, (C₆-C₁₀ aryl)-C₁-C₆ alkyl, (C₆-C₁₀ aryl)-C₂-C₆ alkenyl, (C₆-C₁₀ aryloxy)-C₁-C₆ alkyl, 5- to 10-membered heteroaryl wherein the heteroaryl moiety comprises one to five heteroatoms independently selected from N, O and S; (5- to 10-membered heteroaryl)-(C₁-C₆) alkyl wherein the heteroaryl moiety comprises one to five heteroatoms independently selected from N, O and S, (5- to 10-membered heteroaryl)-(C₂-C₆) alkenyl wherein the heteroaryl moiety comprises one to five heteroatoms independently selected from N, O and S, (5- to 10-membered heteroaryloxy)-(C₁-C₆) alkyl wherein the heteroaryl moiety comprises one to five heteroatoms independently selected from N, O and S, 4- to 12-membered heterocycloalkyl wherein said heterocycloalkyl moiety comprises one to four heteroatoms independently selected from N, O and S(O)ₙ, and (4- to 12-membered heterocycloalkyl)-C₁-C₆ alkyl wherein said heterocycloalkyl moiety comprises one to four heteroatoms independently selected from N, O and S(O)ₙ; wherein each R¹ is optionally independently substituted with one to four R^{1A};
R^{1A} at each occurrence is independently selected from the group consisting of halo, hydroxy, cyano, phenyl, benzyl, amino, (C₁-C₆ alkyl)amino optionally substituted with one to five fluoro, di(C₁-C₆ alkyl)amino optionally substituted with one to ten fluoro, C₁-C₆ alkyl optionally substituted with one to five fluoro, C₁-C₆ alkoxy optionally substituted with one to five fluoro, C₁-C₃ alkoxy-C₁-C₃ alkyl optionally substituted with one to five fluoro, C₃-C₆ cycloalkyl optionally substituted with one to three fluoro or C₁-C₃ alkyl;
R² at each occurrence is independently hydroxy or oxo;
p is 0, 1 or 2;
q and q′ are each independently 0, 1 or 2;
Ring A is an azetidine, pyrrolidine, piperidine or azepane ring which is optionally substituted with one to four R^{A};
R^{A} at each occurrence is independently selected from the group consisting of fluoro, hydroxy, C₁-C₆ alkyl optionally substituted with one to three fluoro or hydroxy and C₁-C₆ alkoxy optionally substituted with one to three fluoro or hydroxy; or two R^{A} groups when attached to adjacent carbons and taken together with the carbons to which they are attached are a fused C₃-C₆ cycloalkyl which is optionally substituted with one to four R^{A2}; or two R^{A} groups when attached to the same carbon and taken together with the carbon to which they are attached are a spiro C₃-C₆ cycloalkyl which is optionally substituted with one to four R^{A2};
R^{A2} at each occurrence is independently selected from fluoro, hydroxy, C₁-C₃ alkyl optionally independently substituted with one to three fluoro or hydroxy and C₁-C₃ alkoxy optionally independently substituted with one to three fluoro or hydroxy;
R³ is selected from the group consisting of C₁-C₈ alkyl, C₁-C₈ alkoxy, (C₁-C₆ alkoxy)-C₁-C₆ alkyl, C₂-C₆ alkynyl, C₂-C₆ alkynyloxy, C₃-C₁₂ cycloalkyl optionally fused with a 5- to 6-membered heteroaryl or phenyl, (C₃-C₁₂ cycloalkyl)-C₁-C₆ alkyl, C₃-C₁₂ cycloalkoxy, (C₃-C₁₂ cycloalkoxy)-C₁-C₆ alkyl, 4- to 12-membered heterocycloalkyl which is optionally fused with a 5- to 6-membered heteroaryl or phenyl and wherein said heterocycloalkyl comprises one to four heteroatoms independently selected from N, O and S(O)ₙ, (4- to 12-membered heterocycloalkyl)-C₁-C₆ alkyl wherein said heterocycloalkyl moiety comprises one to four heteroatoms independently selected from N, O and S(O)ₙ, C₆-C₁₀ aryl optionally fused with a C₄-C₆ cycloalkyl or a 4- to 7-membered heterocycloalkyl, (C₆-C₁₀ aryl)-C₁-C₆ alkyl, (C₆-C₁₀ aryloxy)-C₁-C₆ alkyl, (C₆-C₁₀ aryl)-(C₂-C₆) alkenyl, 5- to 10-membered heteroaryl comprising one to five heteroatoms independently selected from N, O and S, which is optionally fused with a C₅-C₆ cycloalkyl; (5- to 10-membered heteroaryl)-C₁-C₆ alkyl wherein the heteroaryl moiety comprises one to five heteroatoms independently selected from N, O and S; (5- to 10-membered heteroaryl)-(C₂-C₆) alkenyl wherein the heteroaryl moiety comprises one to five heteroatoms independently selected from N, O and S; (C₆-C₁₀ aryl)-(5- to 10-membered heteroaryl)- wherein the heteroaryl moiety comprises one to five heteroatoms independently selected from N, O and S, (5- to 10-membered heteroaryloxy)-C₁-C₆ alkyl wherein the heteroaryl moiety comprises one to five heteroatoms independently selected from N, O and S; (5- to 6-membered heteroaryl)-(5- to 6-membered heteroaryl)- wherein each heteroaryl moiety comprises one to four heteroatoms independently selected from N, O and S; (4- to 7-membered heterocycloalkyl)-(5- to 6-membered heteroaryl)- wherein the heterocycloalkyl moiety comprises one to three heteroatoms independently selected from N, O and S(O)ₙ and the heteroaryl moiety comprises one to four heteroatoms independently selected from N, O and S; (5- to 6-membered heteroaryl)-(4- to 7-membered heterocycloalkyl)- wherein the heterocycloalkyl moiety comprises one to three heteroatoms independently selected from N, O and S(O)ₙ and the heteroaryl moiety comprises one to four heteroatoms independently selected from N, O and S; wherein each R³ group is optionally substituted with one to five R⁴;
R⁴ at each occurrence is independently selected from the group consisting of oxo, halo, hydroxy, cyano, phenyl, benzyl, amino, (C₁-C₆ alkyl)amino optionally substituted with one to five fluoro, di(C₁-C₆ alkyl)amino optionally substituted with one to ten fluoro, C₁-C₆ alkyl optionally substituted with one to five fluoro, C₁-C₆ alkoxy optionally substituted with one to five fluoro, C₁-C₃ alkoxy-C₁-C₃ alkyl optionally substituted with one to five fluoro, C₃-C₆ cycloalkyl optionally substituted with one to three fluoro or C₁-C₃ alkyl, C₁-C₆ alkyl-C(O)NH— optionally substituted with one to five fluoro or with one R⁵, C₁-C₆ alkyl-OC(O)NH— optionally substituted with one to five fluoro or with one R⁵, C₁-C₆ alkyl-NHC(O)NH— optionally substituted with one to five fluoro or with one R⁵, C₁-C₆ alkyl-S(O)₂NH— optionally substituted with one to five fluoro or with one R⁵, C₁-C₆ alkyl-C(O)— optionally substituted with one to five fluoro or with one R⁵, C₁-C₆ alkyl-S(O)ₙ— optionally substituted with one to five fluoro or with one R⁵;
R⁵ is selected from phenyl, phenoxy, C₃-C₆ cycloalkyl, C₃-C₆ cycloalkoxy, 4- to 7-membered heterocycloalkyl- wherein the heterocycloalkyl moiety comprises one to three heteroatoms independently selected from N, O and S(O)ₙ and 5- to 6-membered heteroaryl- wherein the heteroaryl moiety comprises one to four heteroatoms independently selected from N, O and S; wherein each R⁵ is optionally independently substituted with one to three halo, C₁-C₃ alkyl and C₁-C₃ alkoxy; and n at each occurrence is independently selected from 0, 1 and 2.

E2 is the compound of E1 wherein q is 0 and q′ is 1; or a pharmaceutically acceptable salt thereof.

E3 is the compound of E2 of Formula Ia

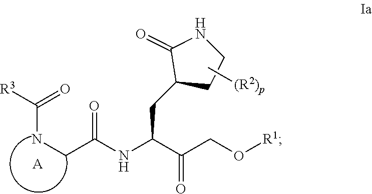

or a pharmaceutically acceptable salt thereof.
E4 is the compound of any one of E1 to E3 wherein Ring A is a pyrrolidine or piperidine ring which is optionally substituted with one to four R^{A}; or a pharmaceutically acceptable salt thereof.

E5 is the compound of any one of E1 to E4 wherein R^{A} at each occurrence is independently selected from the group consisting of fluoro, methyl, isopropyl, trifluoromethyl, tert-butyl and tert-butoxy; or two R^{A} groups when attached to adjacent carbons and taken together with the carbons to which they are attached are a fused cyclopentane or cyclopropane ring which is optionally substituted with one to four R^{A2}; or two R^{A} groups when attached to the same carbon and taken together with the carbon to which they are attached are a spirocyclopropane or spirocyclopentane ring which is optionally substituted with one to four R^{A2}; or a pharmaceutically acceptable salt thereof.

E6 is the compound of E5 wherein R^{A2} at each occurrence is independently selected from the group consisting of fluoro, methyl and methoxy; or a pharmaceutically acceptable salt thereof.

E7 is the compound of E3 selected from the group consisting of formulae Ia-1 through Ia-8

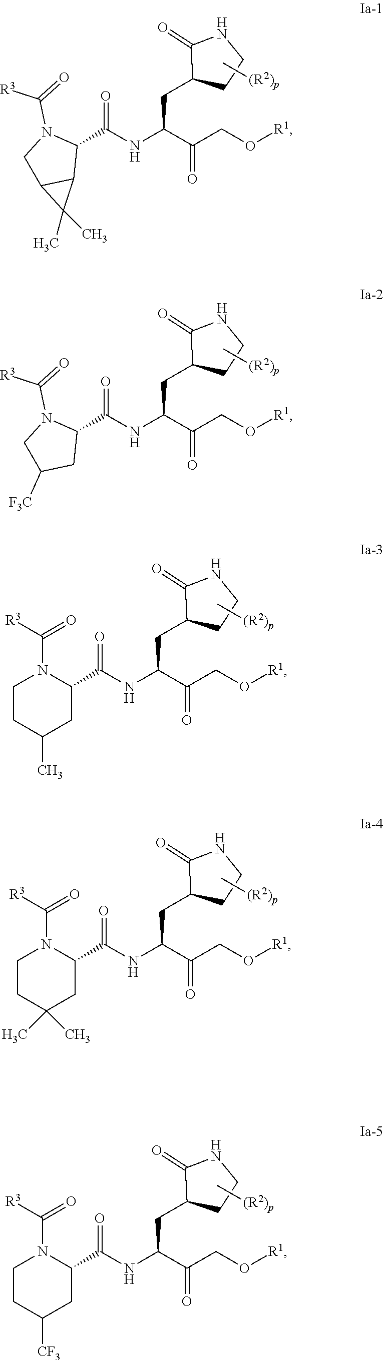

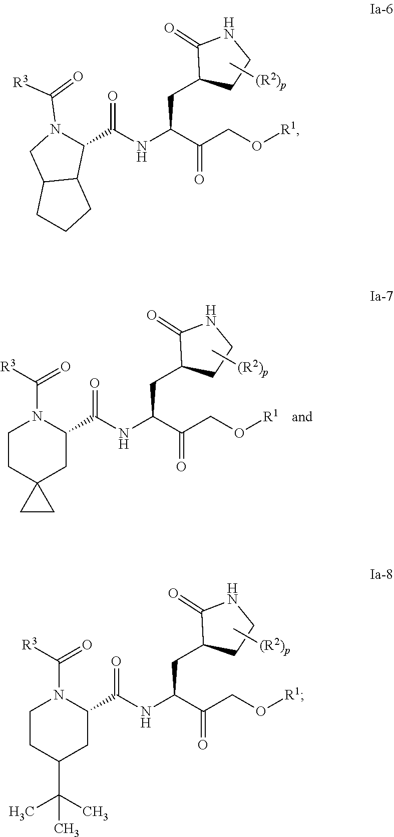

or a pharmaceutically acceptable salt thereof.

E8 is the compound of E7 selected from the group consisting of Ia-1′ through Ia-8″

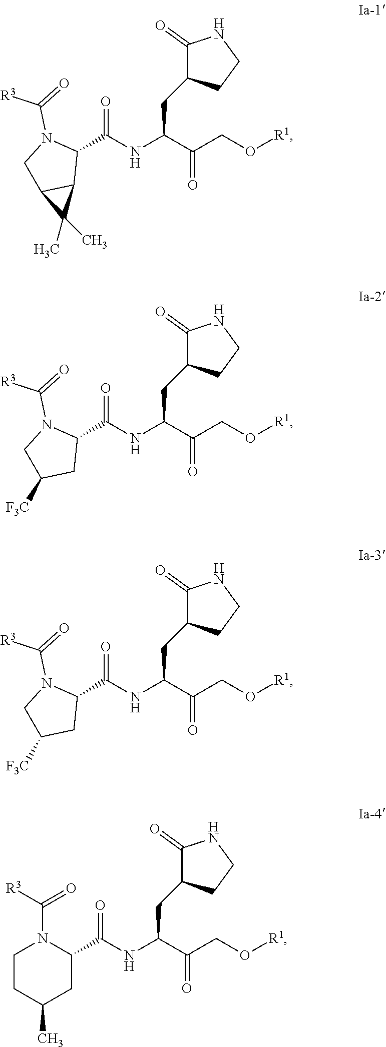

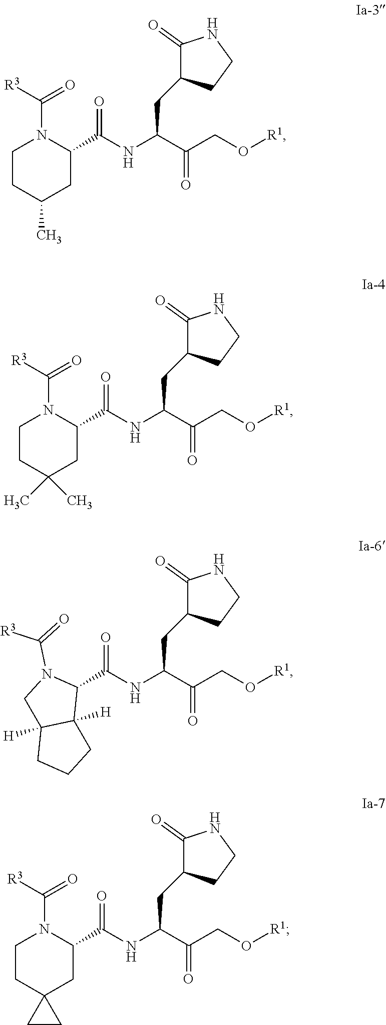

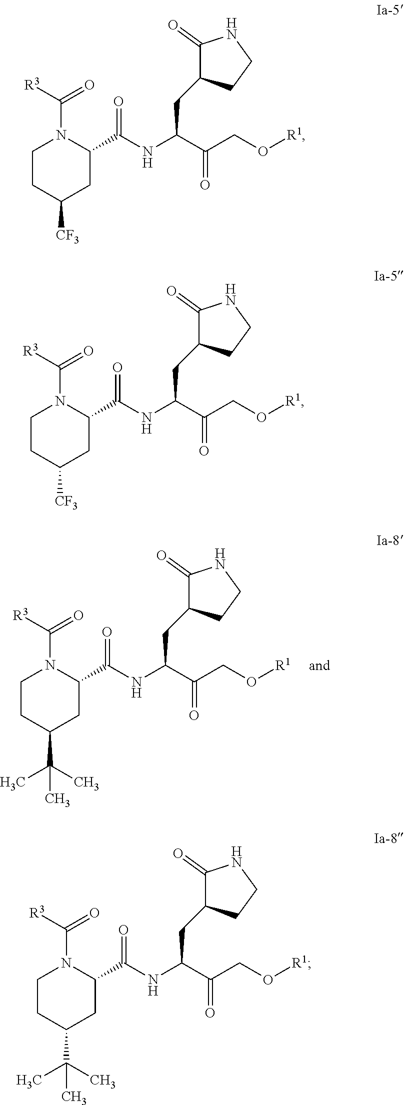

or a pharmaceutically acceptable salt thereof.

E9 is the compound of any one of E1 to E8 wherein R³ is substituted with one R⁴ selected from the group consisting of C₁-C₆ alkyl-C(O)NH— optionally substituted with one to five fluoro or with one R⁵, C₁-C₆ alkyl-OC(O)NH— optionally substituted with one to five fluoro or with one R⁵, C₁-C₆ alkyl-NHC(O)NH— optionally substituted with one to five fluoro or with one R⁵, C₁-C₆ alkyl-S(O)₂NH— optionally substituted with one to five fluoro or with one R⁵, C₁-C₆ alkyl-C(O)— optionally substituted with one to five fluoro or with one R⁵, C₁-C₆ alkyl-S(O)ₙ— optionally substituted with one to five fluoro or with one R⁵; and the R³ is optionally substituted with one to three R⁴; or a pharmaceutically acceptable salt thereof.

E10 is the compound of E9 wherein R³ is selected from the group consisting of C₁-C₈ alkyl, (C₁-C₆ alkoxy)-C₁-C₆ alkyl, C₃-C₇ cycloalkyl and (C₃-C₇ cycloalkyl)-C₁-C₆ alkyl, each of which is substituted with one R⁴ selected from the group consisting of CF₃S(O)₂NH—, CH₃S(O)₂NH—, CF₃C(O)NH—, CH₃C(O)NH— and CH₃OC(O)NH—, and is optionally substituted with one to three R⁴; or a pharmaceutically acceptable salt thereof.

E11 is the compound of E10 wherein R³ is selected from the group consisting of

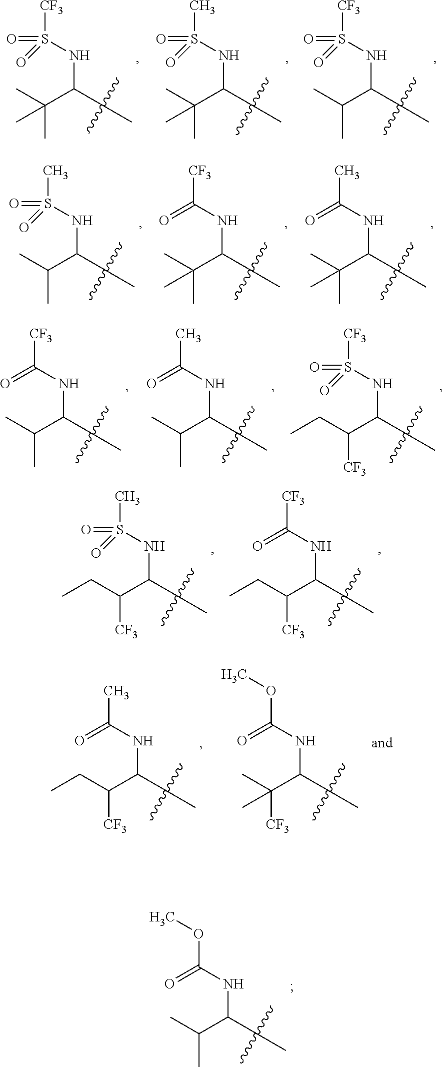

or a pharmaceutically acceptable salt thereof.

E12 is the compound of any one of E1 to E11 wherein R¹ is phenyl which is optionally substituted with one to three R^{1A}; or a pharmaceutically acceptable salt thereof.

E13 is the compound of any one of E1 to E11 wherein R¹ is benzyl which is optionally substituted with one to three R^{1A}; or a pharmaceutically acceptable salt thereof.

E14 is the compound of any one of E1 to E11 wherein R¹ is a 5- to 10-membered heteroaryl wherein the heteroaryl moiety comprises one to four heteroatoms independently selected from N, O and S, and the heteroaryl is optionally substituted with one to three R^{1A}; or a pharmaceutically acceptable salt thereof.

E15 is the compound of E14 wherein R¹ is a 5- to 10-membered heteroaryl selected from the group consisting of pyrrolyl, furanyl, thienyl, imidazolyl, pyrazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl, indolyl, benzoxazolyl, benzimidazolyl, benzothiazolyl, pyridinopyrrolyl, quinolinyl, quinoxalinyl, benzotriazolyl, imidazo[1,2-a]pyridinyl, imidazo[2,1-b][1,3]thiazolyl, 4H-furo[3,2-b]pyrrolyl, 4H-thieno[3,2-b]pyrrolyl, [1,2,4]triazolo[1,5-a]pyrimidinyl, [1,2,3]triazolo[1,5-a]pyridinyl and naphthyridinyl; each of which is optionally substituted with one to three R^{1A}; or a pharmaceutically acceptable salt thereof.

E16 is the compound of any one of E1 to E11 wherein R¹ is C₁-C₆ alkyl which is optionally substituted with one to three R^{1A}; or a pharmaceutically acceptable salt thereof.

E17 is the compound of E16 wherein R¹ is selected from methyl, ethyl, isopropyl, 2,2,2-trifluoroethyl and 1,1,1,3,3,3-hexafluoropropan-2-yl; or a pharmaceutically acceptable salt thereof.

E18 is the compound of any one of E1 to E17 wherein R^{1A} at each occurrence is independently selected from the group consisting of chloro, fluoro, cyano, methyl, difluoromethyl, trifluoromethyl, ethyl, propyl, isopropyl, butyl, tert-butyl, methoxy and trifluoromethoxy; or a pharmaceutically acceptable salt thereof.

E19 is a compound of E1 selected from the group consisting of
(1R,2S,5S)-N-{(2S)-4-[(4-methoxyphenyl)methoxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(2R*,4S*)-4-tert-butyl-N-{(2S)-4-methoxy-3-oxo-1-[(3S)-2-oxopyrrolidin-3-yl]butan-2-yl}-1-[N-(trifluoromethanesulfonyl)-L-valyl]piperidine-2-carboxamide, DIAST-1;
methyl {(2S)-1-[(1R,2S,5S)-2-({(2S)-4-(3-chlorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-6,6-dimethyl-2-({(2S)-3-oxo-1-[(3S)-2-oxopyrrolidin-3-yl]-4-[3-(trifluoromethyl)phenoxy]butan-2-yl}carbamoyl)-3-azabicyclo[3.1.0]hexan-3-yl]-3,3-dimethyl-1-oxobutan-2-yl}carbamate;
(1R,2S,5S)-6,6-dimethyl-N-{(2S)-4-[(6-methylpyridin-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-[(6-methylpyridin-3-yl)oxy]-3-oxo-1-[(3S)-2-oxopyrrolidin-3-yl]butan-2-yl}-3-[3-methyl-N-(trifluoroacetyl)-L-valyl]-3-azabicyclo[3.1.0]hexane-2-carboxamide;
(1R,2S,5S)-6,6-dimethyl-3-[3-methyl-N-(trifluoroacetyl)-L-valyl]-N-{(2S)-3-oxo-1-[(3S)-2-oxopyrrolidin-3-yl]-4-phenoxybutan-2-yl}-3-azabicyclo[3.1.0]hexane-2-carboxamide
methyl {(2S)-1-[(1R,2S,5S)-2-({(2S)-4-(4-fluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-2-({(2S)-4-(2-fluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-6,6-dimethyl-N-{(2S)-3-oxo-1-[(3S)-2-oxopyrrolidin-3-yl]-4-phenoxybutan-2-yl}-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-3-[N-(methanesulfonyl)-3-methyl-L-valyl]-6,6-dimethyl-3-azabicyclo[3.1.0]hexane-2-carboxamide;
methyl {(2S)-1-[(1R,2S,5S)-2-({(2S)-4-(2,4-difluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-2-({(2S)-4-(3-chlorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3-methyl-1-oxobutan-2-yl}carbamate;
(1R,2S,5S)-3-[N-(methanesulfonyl)-3-methyl-L-valyl]-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-[(3-ethyl-1-methyl-1H-pyrazol-5-yl)oxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-[(1-ethyl-3-methyl-1H-pyrazol-5-yl)oxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2R)-4-(4-fluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
methyl {(2S)-1-[(1R,2S,5S)-6,6-dimethyl-2-({(2S)-3-oxo-1-[(3S)-2-oxopyrrolidin-3-yl]-4-phenoxybutan-2-yl}carbamoyl)-3-azabicyclo[3.1.0]hexan-3-yl]-3,3-dimethyl-1-oxobutan-2-yl}carbamate;
(1R,2S,5S)-N-{(2S)-4-[(6-fluoropyridin-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoroacetyl)-L-isoleucyl]-3-azabicyclo[3.1.0]hexane-2-carboxamide;
methyl {(2S)-1-[(1R,2S,5S)-2-({(2S)-4-[(1-ethyl-3-methyl-1H-pyrazol-5-yl)oxy]-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-6,6-dimethyl-3-[3-methyl-N-(trifluoroacetyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-(2,5-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2RS)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
methyl {(2S)-1-[(1R,2S,5S)-6,6-dimethyl-2-({(2S)-4-{[1-methyl-3-(trifluoromethyl)-1H-pyrazol-5-yl]oxy}-3-oxo-1-[(3S)-2-oxopyrrolidin-3-yl]butan-2-yl}carbamoyl)-3-azabicyclo[3.1.0]hexan-3-yl]-3-methyl-1-oxobutan-2-yl}carbamate;
methyl {(2S)-1-[(1R,2S,5S)-2-({(2S)-4-[(3-ethyl-1-methyl-1H-pyrazol-5-yl)oxy]-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoroacetyl)-L-valyl]-N-{(2S)-3-oxo-1-[(3S)-2-oxopyrrolidin-3-yl]-4-[3-(trifluoromethyl)phenoxy]butan-2-yl}-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoromethanesulfonyl)-L-valyl]-3-azabicyclo[3.1.0]hexane-2-carboxamide;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[N-(trifluoromethanesulfonyl)-L-valyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4-(trifluoromethyl)piperidine-2-carboxamide, DIAST-2;
(1R,2S,5S)-N-{(2S)-4-(2,4-difluorophenoxy)-1-[(3R)-2,5-dioxopyrrolidin-3-yl]-3-oxobutan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[3-(trifluoromethyl)phenoxy]butan-2-yl}-3-azabicyclo[3.1.0]hexane-2-carboxamide;
(1R,2S,5S)-N-{(2S)-4-[(1-ethyl-3-methyl-1H-pyrazol-5-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-1-[(3R)-2,5-dioxopyrrolidin-3-yl]-3-oxobutan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-[(3-ethyl-1-methyl-1H-pyrazol-5-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(4-methyl-1,2,5-thiadiazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[3-(trifluoromethyl)phenoxy]butan-2-yl}-3-[N-(trifluoromethanesulfonyl)-L-valyl]-3-azabicyclo[3.1.0]hexane-2-carboxamide;
(1R,2S,5S)-N-{(2S)-4-(2-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(3,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-[N-(trifluoromethanesulfonyl)-L-valyl]-3-azabicyclo[3.1.0]hexane-2-carboxamide;
(1R,2S,5S)-N-{(2S)-4-(2,4-difluorophenoxy)-1-[(3R)-2,5-dioxopyrrolidin-3-yl]-3-oxo butan-2-yl}-6,6-dimethyl-3-[N-(trifluoromethanesulfonyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(6-methyl-1,2-benzoxazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(4-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,5-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[N-(trifluoromethanesulfonyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-1,2,4-triazol-5-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[3-(propan-2-yl)phenoxy]butan-2-yl}-3-[N-(trifluoroacetyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoroacetyl)-L-isoleucyl]-3-azabicyclo[3.1.0]hexane-2-carboxamide;
(1R,2S,5S)-N-{(2S)-4-(3,5-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(5R*)-N-{(2S)-4-(2,4-difluorophenoxy)-3-oxo-1-[(3S)-2-oxopyrrolidin-3-yl]butan-2-yl}-6-[N-(methanesulfonyl)-L-valyl]-6-azaspiro[2.5]octane-5-carboxamide, DIAST-1;
(2R*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4,4-dimethylpiperidine-2-carboxamide;
(1R,2S,5S)-6,6-dimethyl-N-{(2S)-4-[(5-methyl-1,2-oxazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[(1,2,5-thiadiazol-3-yl)oxy]butan-2-yl}-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoromethanesulfonyl)-L-isoleucyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[(1-phenyl-1H-1,2,4-triazol-5-yl)oxy]butan-2-yl}-3-[N-(trifluoroacetyl)-L-valyl]-3-azabicyclo[3.1.0]hexane-2-carboxamide;
(1R,2S,5S)-6,6-dimethyl-3-[N-methyl-N-(propan-2-yl)-L-leucyl]-N-{(2R*)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide, DIAST-1;
(5R*)-N-{(2S)-4-(2,4-difluorophenoxy)-3-oxo-1-[(3S)-2-oxopyrrolidin-3-yl]butan-2-yl}-6-[N-(methanesulfonyl)-L-valyl]-6-azaspiro[2.5]octane-5-carboxamide, DIAST-2;
(1R,2S,5S)-N-{(2S)-4-[(4-ethyl-5-methyl-1,2-oxazol-3-yl)oxy]-3-oxo-1-[(3S)-2-oxo pyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2R)-4-(2,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2R)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-4-methyl-1-[N-(trifluoroacetyl)-L-valyl]piperidine-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoromethanesulfonyl)-L-isoleucyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4-(trifluoromethyl)piperidine-2-carboxamide, DIAST-1;
(1R,2S,5S)-6,6-dimethyl-N-{(2S)-4-[(6-methyl-1,3-benzoxazol-2-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-3-[N-methyl-N-(propan-2-yl)-L-leucyl]-N-{(2R*)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide, DIAST-2;
(1R,2S,5S)-6,6-dimethyl-N-{(2S)-4-{[4-methyl-5-(trifluoromethyl)-4H-1,2,4-triazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-[(8-fluoro[1,2,4]triazolo[4,3-a]pyridin-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(3-methyl-1,2,4-oxadiazol-5-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(2R*,4S*)-4-tert-butyl-N-{(2S)-4-methoxy-3-oxo-1-[(3S)-2-oxopyrrolidin-3-yl]butan-2-yl}-1-[N-(trifluoromethanesulfonyl)-L-valyl]piperidine-2-carboxamide, DIAST-2;
(1R,2S,5S)-N-{(2S)-4-[(5-tert-butyl-4-methyl-4H-1,2,4-triazol-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[4-methyl-5-(propan-2-yl)-4H-1,2,4-triazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-(2-methylphenoxy)-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(3-cyanophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-[(5-tert-butyl-4-methyl-1,2-oxazol-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-5-(trifluoromethyl)-1H-pyrazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-[(5-fluoro-1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-methoxy-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-methoxy-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
methyl {(2S)-1-[(1R,2S,5S)-6,6-dimethyl-2-({(2S)-4-{[1-methyl-3-(trifluoromethyl)-1H-pyrazol-5-yl]oxy}-3-oxo-1-[(3S)-2-oxopyrrolidin-3-yl]butan-2-yl}carbamoyl)-3-azabicyclo[3.1.0]hexan-3-yl]-3,3-dimethyl-1-oxobutan-2-yl}carbamate;
methyl {(2S)-1-[(1R,2S,5S)-6,6-dimethyl-2-({(2S)-4-[(6-methylpyridin-3-yl)oxy]-3-oxo-1-[(3S)-2-oxopyrrolidin-3-yl]butan-2-yl}carbamoyl)-3-azabicyclo[3.1.0]hexan-3-yl]-3,3-dimethyl-1-oxobutan-2-yl}carbamate;
(1R,2S,5S)-N-{(2S)-4-(2-fluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
methyl {(2S)-1-[(1R,2S,5S)-2-({(2S)-4-(2,5-difluorophenoxy)-3-oxo-1-[(3S)-2-oxo pyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-N-{(2S)-4-methoxy-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-N-{(2S)-4-[(2-methylpyridin-4-yl)oxy]-3-oxo-1-[(3S)-2-oxo pyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(5-chloro-2-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-{[5-(difluoromethyl)-1-methyl-1H-pyrazol-3-yl]oxy}-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(2S,4R)-4-tert-butyl-N-{(2S)-4-methoxy-3-oxo-1-[(3S)-2-oxopyrrolidin-3-yl]butan-2-yl}-1-[3-methyl-N-(trifluoromethanesulfonyl)-L-valyl]piperidine-2-carboxamide;
3-methyl-N-(trifluoromethanesulfonyl)-L-valyl-(4R)-N-{(2S)-4-(2,4-difluorophenoxy)-3-oxo-1-[(3S)-2-oxopyrrolidin-3-yl]butan-2-yl}-4-(trifluoromethyl)-L-prolinamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[3-(trifluoromethoxy)phenoxy]butan-2-yl}-3-azabicyclo[3.1.0]hexane-2-carboxamide;
(1R,2S,5S)-N-{(2S)-4-(4-cyanophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[(1,2-thiazol-3-yl)oxy]butan-2-yl}-3-azabicyclo[3.1.0]hexane-2-carboxamide;
(1R,2S,5S)-N-{(2S)-4-[(1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-N-{(2S)-4-[(2-methylpyridin-3-yl)oxy]-3-oxo-1-[(3S)-2-oxopyrrolidin-3-yl]butan-2-yl}-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-phenoxybutan-2-yl}-3-azabicyclo[3.1.0]hexane-2-carboxamide;
(1R,2S,5S)-N-{(2S)-4-[(5-chloro-1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-(3-ethylphenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[(1,3-thiazol-2-yl)oxy]butan-2-yl}-3-azabicyclo[3.1.0]hexane-2-carboxamide;
(1R,2S,5S)-N-{(2S)-4-[(1-ethyl-1H-pyrazol-3-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
3-methyl-N-(trifluoromethanesulfonyl)-L-valyl-(4R)-N-{(2S)-4-(3-chlorophenoxy)-3-oxo-1-[(3S)-2-oxopyrrolidin-3-yl]butan-2-yl}-4-(trifluoromethyl)-L-prolinamide;
3-methyl-N-(trifluoromethanesulfonyl)-L-valyl-(4R)-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-4-(trifluoromethyl)-L-prolinamide;
(1R,2S,5S)-N-{(2S)-4-(3,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2R)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(2S,4R)-4-tert-butyl-1-{(2S)-2-cyclopentyl-2-[(trifluoromethanesulfonyl)amino]acetyl}-N-{(2S)-4-methoxy-3-oxo-1-[(3S)-2-oxopyrrolidin-3-yl]butan-2-yl}piperidine-2-carboxamide;
(1S,3aR,6aS)-N-{(2S)-4-methoxy-3-oxo-1-[(3S)-2-oxopyrrolidin-3-yl]butan-2-yl}-2-{(2S)-3-methyl-2-[(trifluoromethanesulfonyl)amino]butanoyl}octahydrocyclopenta[c]pyrrole-1-carboxamide; and
(1S,3aR,6aS)-2-{(2S)-3,3-dimethyl-2-[(trifluoromethanesulfonyl)amino]butanoyl}-N-{(2S)-4-methoxy-3-oxo-1-[(3S)-2-oxopyrrolidin-3-yl]butan-2-yl}octahydrocyclopenta[c]pyrrole-1-carboxamide;
   or a pharmaceutically acceptable thereof.

E20 is a pharmaceutical composition comprising a therapeutically effective amount of a compound according to any one of E1 to E19; or a pharmaceutically acceptable salt thereof together with a pharmaceutically acceptable carrier.

E21 is a method of treating a coronavirus infection in a patient, the method comprising administering a therapeutically effective amount of a compound according to any one of E1 to E19; or a pharmaceutically acceptable salt thereof.

E22 is the method of E21 wherein the coronavirus infection is COVID-19.

E23 is the method of E21 or E22 wherein the compound; or a pharmaceutically acceptable salt thereof is administered orally.

E24 is the method of any one of E21 to E23 further comprising administration of one or more additional therapeutic agents.

E25 is the method of E24 wherein the one or more additional therapeutic agent is selected from the group consisting of remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, ABX464, dexamethasone, hydrocortisone, convalescent plasma, gelsolin (Rhu-p65N), regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, casirivimab/imdevimab (REGN-Cov2), MK-7110 (CD24Fc/SACCOVID), heparin, apixaban, tocilizumab (Actemra), sarilumab (Kevzara), apilimod dimesylate, DNL758, DC402234, PB1046, dapaglifozin, abivertinib, ATR-002, bemcentinib, acalabrutinib, baricitinib, tofacitinib, losmapimod, famotidine, niclosamide and diminazene.

E26 is the method of E25 wherein the one or more additional therapeutic agent is selected from the group consisting of remdesivir, dexamethasone, bamlanivimab, casirivimab/imdevimab, tofacitinib and baricitinib.

E27 is a compound according to any one of E1 to E19; or a pharmaceutically acceptable salt thereof for use in the treatment of a coronavirus.

E28 is the compound for use according to E27 wherein the coronavirus infection is COVID-19.

E29 is the compound for use according to E27 or E28 wherein the compound; or a pharmaceutically acceptable salt thereof is administered orally.

E30 is the compound for use according to E27 or E28 further comprising administration of one or more additional therapeutic agents.

E31 is the compound for use according to claim 30 wherein the one or more additional therapeutic agent is selected from the group consisting of remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, ABX464, dexamethasone, hydrocortisone, convalescent plasma, gelsolin (Rhu-p65N), regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, casirivimab/imdevimab (REGN-Cov2), MK-7110 (CD24Fc/SACCOVID), heparin, apixaban, tocilizumab (Actemra), sarilumab (Kevzara), apilimod dimesylate, DNL758, DC402234, PB1046, dapaglifozin, abivertinib, ATR-002, bemcentinib, acalabrutinib, baricitinib, tofacitinib, losmapimod, famotidine, niclosamide and diminazene.

E32 is the compound for use according to E31 wherein the one or more additional therapeutic agent is selected from the group consisting of remdesivir, dexamethasone, bamlanivimab, casirivimab/imdevimab, baricitinib and tofacitinib.

Another embodiment of the invention is a method of inhibiting or preventing SARS-CoV-2 viral replication comprising contacting the SARS-CoV-2 coronavirus 3CL protease with a therapeutically effective amount of a compound or a pharmaceutically acceptable salt thereof of any one of E1 to E19.

Another embodiment of the invention is a method of inhibiting or preventing SARS-CoV-2 viral replication in a patient comprising administering to the patient in need of inhibition of or prevention of SARS-CoV-2 viral replication a therapeutically effective amount of a compound or a pharmaceutically acceptable salt thereof of any one of E1 to E19.

Another embodiment of the invention is the use of a compound or a pharmaceutically acceptable salt thereof of any one of E1 to E19 for the treatment of a coronavirus infection. Another embodiment of the invention is the use of the immediately preceding embodiment wherein the coronavirus infection is COVID-19.

Another embodiment of the invention is the use of a compound or a pharmaceutically acceptable salt thereof of any one of E1 to E19 for the preparation of a medicament that is useful for the treatment of a coronavirus infection. The use of the immediately preceding embodiment wherein the coronavirus infection is COVID-19.

Another embodiment of the present invention is a method of treating a coronavirus infection in a patient, the method comprising administering a therapeutically effective amount of a compound of any one of E1 to E19 to a patient in need thereof.

Another embodiment of the present invention is the method of the immediately preceding embodiment wherein the coronavirus infection is COVID-19.

Another embodiment of the present invention is a method of treating a coronavirus infection in a patient, the method comprising administering a therapeutically effective amount of a compound any one of E1 to E19 wherein an additional therapeutic agent is administered and the additional therapeutic agent is selected from the group consisting of remdesivir, galidesivir, favilavir/avifavir, molnupiravir, AT-527, AT-301, BLD-2660, favipiravir, camostat, SLV213, emtrictabine/tenofivir, clevudine, dalcetrapib, boceprevir, ABX464, dexamethasone, hydrocortisone, convalescent plasma, gelsolin (Rhu-p65N), regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, casirivimab/imdevimab (REGN-Cov2), MK-7110 (CD24Fc/SACCOVID), heparin, apixaban, tocilizumab (Actemra), sarilumab (Kevzara), apilimod dimesylate, DNL758, DC402234, PB1046, dapaglifozin, abivertinib, ATR-002, bemcentinib, acalabrutinibbaricitinib, tofacitinib, losmapimod, famotidine, ritonavir, niclosamide and diminazene.

The present invention also provides a method of targeting SARS-CoV-2 inhibition as a means of treating indications caused by SARS-CoV-2-related viral infections.

The present invention also provides a method of identifying cellular or viral pathways interfering with the functioning of the members of which could be used for treating indications caused by SARS-CoV-2 infections by administering a SARS-CoV-2 protease inhibitor of Formula I as described herein.

The present invention also provides a method of using SARS-CoV-2 protease inhibitors of Formula I as described herein as tools for understanding mechanism of action of other SARS-CoV-2 inhibitors.

The present invention also provides a method of using SARS-CoV-2 3C-like protease inhibitors of Formula I for carrying out gene-profiling experiments for monitoring the up- or down-regulation of genes for the purpose of identifying inhibitors for treating indications caused by SARS-CoV-2 infections such as COVID-19.

The present invention further provides a pharmaceutical composition for the treatment of COVID-19 in a mammal containing an amount of a SARS-CoV-2 3C-like protease inhibitor of any one of E1 to E19 that is effective in treating COVID-19 together with a pharmaceutically acceptable carrier.

Another embodiment of the present invention is a method of treating MERS in a patient, the method comprising administering a therapeutically effective amount of a compound of any one of E1 to 19 to a patient in need thereof.

Another embodiment of the invention is a method of treating MERS in a patient, the method comprising administering a pharmaceutical composition comprising a compound of any one of E1 to E19 to a patient in need thereof.

Another embodiment of the present invention is a method of inhibiting or preventing coronavirus viral replication in a patient comprising administering to the patient in need of inhibition of or prevention of coronavirus viral replication a therapeutically effective amount of a compound of any one of E1 to E19 or a pharmaceutically acceptable salt thereof.

### DETAILED DESCRIPTION OF THE INVENTION

For the purposes of the present invention, as described and claimed herein, the following terms are defined as follows:

As used herein, the terms “comprising” and “including” are used in their open, non-limiting sense. The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “alkyl” as used herein refers to a linear or branched-chain saturated hydrocarbyl substituent (i.e., a substituent obtained from a hydrocarbon by removal of a hydrogen); in one embodiment containing from one to eight carbon atoms, in another one to six carbon atoms and in yet another one to three carbon atoms. Non-limiting examples of such substituents include methyl, ethyl, propyl (including n-propyl and isopropyl), butyl (including n-butyl, isobutyl, sec-butyl and tert-butyl), pentyl, isoamyl, hexyl, heptyl, octyl and the like. In another embodiment containing one to three carbons and consisting of methyl, ethyl, n-propyl and isopropyl.

The term “alkenyl” as used herein refers to a linear or branched-chain unsaturated hydrocarbyl substituent that contains a carbon-carbon double bond (i.e., a substituent obtained from a double bond-containing hydrocarbon by removal of a hydrogen); in one embodiment containing from two to six carbon atoms. Non-limiting examples of such substituents include vinyl, prop-2-en-1-yl, but-3-en-1-yl, pent-4-en-1-yl and hex-5-en-1-yl. When a vinyl group is attached to another group, for example a phenyl group, the moiety formed can be a styryl group.

The term “alkynyl” as used herein refers to a linear or branched-chain unsaturated hydrocarbyl substituent that contains a carbon-carbon triple bond (i.e., a substituent obtained from a triple bond-containing hydrocarbon by removal of a hydrogen); in one embodiment containing from two to six carbon atoms. Non-limiting examples of such substituents include prop-2-yn-1-yl, but-3-yn-1-yl, pent-4-yn-1-yl and hex-5-yn-1-yl.

The term “alkoxy” refers to a linear or branched-chain saturated hydrocarbyl substituent attached to an oxygen radical (i.e., a substituent obtained from a hydrocarbon alcohol by removal of the hydrogen from the OH); in one embodiment containing from one to six carbon atoms. Non-limiting examples of such substituents include methoxy, ethoxy, propoxy (including n-propoxy and isopropoxy), butoxy (including n-butoxy, isobutoxy, sec-butoxy and tert-butoxy), pentoxy, hexoxy and the like. In another embodiment having one to three carbons and consisting of methoxy, ethoxy, n-propoxy and isopropoxy. An alkoxy group which is attached to an alkyl group is referred to as an alkoxyalkyl. An example of an alkoxyalkyl group is methoxymethyl.

The term “alkynyloxy” refers to a linear or branched-chain unsaturated hydrocarbyl substituent containing a carbon-carbon triple bond attached to an oxygen radical (i.e., a substituent obtained from a triple bond-containing hydrocarbon alcohol by removal of the hydrogen from the OH); in one embodiment containing from three to six carbon atoms. Non-limiting examples of such substituents include propynyloxy, butynyloxy and pentynyloxy and the like.

In some instances, the number of carbon atoms in a hydrocarbyl substituent (i.e., alkyl, cycloalkyl, etc.) is indicated by the prefix “Cₓ-C_{y}-” or “C_{x-y}”, wherein x is the minimum and y is the maximum number of carbon atoms in the substituent. Thus, for example, “C₁-C₈ alkyl” or “C₁₋₈ alkyl” refers to an alkyl substituent containing from 1 to 8 carbon atoms, “C₁-C₆ alkyl” or “C₁₋₆ alkyl” refers to an alkyl substituent containing from 1 to 6 carbon atoms, “C₁-C₃ alkyl” or “C₁₋₃ alkyl” refers to an alkyl substituent containing from 1 to 3 carbon atoms. Illustrating further, C₃-C₆ cycloalkyl or C₃₋₆-cycloalkyl refers to a saturated cycloalkyl group containing from 3 to 6 carbon ring atoms.

The term “cycloalkyl” refers to a carbocyclic substituent obtained by removing a hydrogen from a saturated carbocyclic molecule, for example one having three to seven carbon atoms. The term “cycloalkyl” includes monocyclic saturated carbocycles. The term “C₃-C₇ cycloalkyl” means a radical of a three- to seven-membered ring system which includes the groups cyclopropyl, cyclobutyl, cyclopentyl, cyclohexyl, and cycloheptyl. The term “C₃-C₆ cycloalkyl” means a radical of a three- to six-membered ring system which includes the groups cyclopropyl, cyclobutyl, cyclopentyl and cyclohexyl. The cycloalkyl groups can also be bicyclic or spirocyclic carbocycles. For example, the term “C₃-C₁₂ cycloalkyl” includes monocyclic carbocycles and bicyclic and spirocyclic cycloalkyl moieties such as bicyclopentyl, bicyclohexyl, bicycloheptyl, bicyclooctyl, bicyclononyl, spiropentyl, spirohexyl, spiroheptyl, spirooctyl and spirononyl.

The term “C₃-C₆ cycloalkoxy” refers to a three- to six-membered cycloalkyl group attached to an oxygen radical. Examples include cyclopropoxy, cyclobutoxy, cyclopentoxy and cyclohexoxy.

The term “aryl” refers to a carbocyclic aromatic system. The term “C₆-C₁₀ aryl” refers to carbocyclic aromatic systems with 6 to 10 atoms and includes phenyl and naphthyl. The term “C₆-C₁₀ aryloxy” is a 6 to 10 atom aromatic carbocycle linked to an oxygen radical, and includes groups such as phenoxy and naphthyloxy.

In some instances, the number of atoms in a cyclic substituent containing one or more heteroatoms (i.e., heteroaryl or heterocycloalkyl) is indicated by the prefix “x- to y-membered”, wherein x is the minimum and y is the maximum number of atoms forming the cyclic moiety of the substituent. Thus, for example, “4- to 6-membered heterocycloalkyl” refers to a heterocycloalkyl containing from 4 to 6 atoms, including one to three heteroatoms, in the cyclic moiety of the heterocycloalkyl. Likewise, the phrase “5- to 6-membered heteroaryl” refers to a heteroaryl containing from 5 to 6 atoms, and “5- to 10-membered heteroaryl” refers to a heteroaryl containing from 5 to 10 atoms, each including one or more heteroatoms, in the cyclic moiety of the heteroaryl. Furthermore, the phrases “5-membered heteroaryl” and “6-membered heteroaryl” refer to a five-membered heteroaromatic ring system and a six-membered heteroaromatic ring system, respectively. The heteroatoms present in these ring systems are selected from N, O and S.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol. The terms cyano and nitrile refer to a —CN group. The term “oxo” means an oxygen which is attached to a carbon by a double bond (i.e., when R⁴ is oxo then R⁴ together with the carbon to which it is attached are a C═O moiety).

The term “halo” or “halogen” refers to fluorine (which may be depicted as —F), chlorine (which may be depicted as —Cl), bromine (which may be depicted as —Br), or iodine (which may be depicted as —I).

The term “heterocycloalkyl” refers to a substituent obtained by removing a hydrogen from a saturated or partially saturated ring structure containing a total of the specified number of atoms, such as 4 to 6 ring atoms or 4 to 12 atoms, wherein at least one of the ring atoms is a heteroatom (i.e., oxygen, nitrogen, or sulfur), with the remaining ring atoms being independently selected from the group consisting of carbon, oxygen, nitrogen, and sulfur. The sulfur may be oxidized [i.e., S(O) or S(O)₂] or not. In a group that has a heterocycloalkyl substituent, the ring atom of the heterocycloalkyl substituent that is bound to the group may be a nitrogen heteroatom, or it may be a ring carbon atom. Similarly, if the heterocycloalkyl substituent is in turn substituted with a group or substituent, the group or substituent may be bound to a nitrogen heteroatom, or it may be bound to a ring carbon atom. It is to be understood that a heterocyclic group may be monocyclic, bicyclic, polycyclic or spirocyclic.

The term “heteroaryl” refers to an aromatic ring structure containing the specified number of ring atoms in which at least one of the ring atoms is a heteroatom (i.e., oxygen, nitrogen, or sulfur), with the remaining ring atoms being independently selected from the group consisting of carbon, oxygen, nitrogen, and sulfur. Examples of heteroaryl substituents include 6-membered heteroaryl substituents such as pyridyl, pyrazyl, pyrimidinyl, and pyridazinyl; and 5-membered heteroaryl substituents such as triazolyl, imidazolyl, furanyl, thiophenyl, pyrazolyl, pyrrolyl, oxazolyl, isoxazolyl, thiazolyl, 1,2,3-, 1,2,4-, 1,2,5-, or 1,3,4-oxadiazolyl and isothiazolyl. The heteroaryl group can also be a bicyclic heteroaromatic group such as indolyl, benzofuranyl, benzothienyl, benzimidazolyl, benzothiazolyl, benzoxazolyl, benzoisoxazolyl, oxazolopyridinyl, imidazopyridinyl, imidazopyrimidinyl and the like. In a group that has a heteroaryl substituent, the ring atom of the heteroaryl substituent that is bound to the group may be one of the heteroatoms, or it may be a ring carbon atom. Similarly, if the heteroaryl substituent is in turn substituted with a group or substituent, the group or substituent may be bound to one of the heteroatoms, or it may be bound to a ring carbon atom. The term “heteroaryl” also includes pyridyl N-oxides and groups containing a pyridine N-oxide ring. In addition, the heteroaryl group may contain an oxo group such as the one present in a pyridone group. Further examples include furyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, pyridin-2(1H)-onyl, pyridazin-2(1H)-onyl, pyrimidin-2(1H)-onyl, pyrazin-2(1H)-onyl, imidazo[1,2-a]pyridinyl, and pyrazolo[1,5-a]pyridinyl. The heteroaryl can be further substituted as defined herein.

Examples of single-ring heteroaryls and heterocycloalkyls include furanyl, dihydrofuranyl, tetrahydrofuranyl, thiophenyl, dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaoxadiazolyl, oxathiazolyl, oxadiazolyl (including oxadiazolyl, 1,2,4-oxadiazolyl, 1,2,5-oxadiazolyl, or 1,3,4-oxadiazolyl), pyranyl (including 1,2-pyranyl or 1,4-pyranyl), dihydropyranyl, pyridinyl, piperidinyl, diazinyl (including pyridazinyl, pyrimidinyl, piperazinyl, triazinyl (including s-triazinyl, as-triazinyl and v-triazinyl), oxazinyl (including 2H-1,2-oxazinyl, 6H-1,3-oxazinyl, or 2H-1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 2H-1,2,4-oxadiazinyl or 2H-1,2,5-oxadiazinyl), and morpholinyl.

The term “heteroaryl” can also include, when specified as such, ring systems having two rings wherein such rings may be fused and wherein one ring is aromatic and the other ring is not fully part of the conjugated aromatic system (i.e., the heteroaromatic ring can be fused to a cycloalkyl or heterocycloalkyl ring). Non-limiting examples of such ring systems include 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

The term “heteroaryloxy” means heteroaryl groups as described herein that are attached to an oxygen radical and include groups such as pyridinyloxy, thienyloxy, furanyloxy and the like.

If substituents are described as “independently” having more than one variable, each instance of a substituent is selected independent of the other(s) from the list of variables available. Each substituent therefore may be identical to or different from the other substituent(s).

If substituents are described as being “independently selected” from a group, each instance of a substituent is selected independent of the other(s). Each substituent therefore may be identical to or different from the other substituent(s).

As used herein, the term “Formula I” may be hereinafter referred to as a “compound(s) of the invention,” “the present invention,” and “compound of Formula I.” Such terms are also defined to include all forms of the compound of Formula I, including hydrates, solvates, isomers, crystalline and non-crystalline forms, isomorphs, polymorphs, and metabolites thereof. For example, the compounds of the invention, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist as clathrates or other complexes. Included within the scope of the invention are complexes such as clathrates, drug-host inclusion complexes wherein the drug and host are present in stoichiometric or non-stoichiometric amounts. Also included are complexes of the compounds of the invention containing two or more organic and/or inorganic components, which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

The compounds of the invention have asymmetric carbon atoms. The carbon-carbon bonds of the compounds of the invention may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I can exist as enantiomers and diastereomers or as racemates and mixtures thereof. The use of a solid line to depict bonds to one or more asymmetric carbon atoms in a compound of Formula I and the use of a solid or dotted wedge to depict bonds to other asymmetric carbon atoms in the same compound is meant to indicate that a mixture of diastereomers is present.

Stereoisomers of Formula I include cis and trans isomers, optical isomers such as R and S enantiomers, diastereomers, geometric isomers, rotational isomers, conformational isomers, and tautomers of the compounds of the invention, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I may exhibit the phenomenon of tautomerism; such tautomers are also regarded as compounds of the invention. All such tautomeric forms, and mixtures thereof, are included within the scope of compounds of Formula I. Tautomers exist as mixtures of a tautomeric set in solution. In solid form, usually one tautomer predominates. Even though one tautomer may be described, the present invention includes all tautomers of the compounds of Formula I and salts thereof.

The phrase “pharmaceutically acceptable salts(s)”, as used herein, unless otherwise indicated, includes salts of acidic or basic groups which may be present in the compounds described herein. The compounds used in the methods of the invention that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, hexylresorcinate, hydrabamine, hydrobromide, hydrochloride, iodide, isethionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

With respect to the compounds of the invention used in the methods of the invention, if the compounds also exist as tautomeric forms then this invention relates to those tautomers and the use of all such tautomers and mixtures thereof.

The subject invention also includes compounds and methods of treatment of coronavirus infections such as COVID-19 and methods of inhibiting SARS-CoV-2 with isotopically labelled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or isotopes of other atoms are with the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds used in the methods of this invention and prodrugs thereof can generally be prepared by carrying out the procedures for preparing the compounds disclosed in the art by substituting a readily available isotopically labelled reagent for a non-isotopically labelled reagent.

This invention also encompasses methods using pharmaceutical compositions and methods of treating coronavirus infections such as COVID-19 infections through administering prodrugs of compounds of the invention. Compounds having free amino, amido or hydroxy groups can be converted into prodrugs. Prodrugs include compounds wherein an amino acid residue, or a polypeptide chain of two or more (e.g., two, three or four) amino acid residues is covalently joined through an ester bond to a hydroxy of compounds used in the methods of this invention. The amino acid residues include but are not limited to the 20 naturally occurring amino acids commonly designated by three letter symbols and also include 4-hydroxyproline, hydroxylysine, desmosine, isodesmosine, 3-methylhistidine, norvalin, beta-alanine, gamma-aminobutyric acid, citrulline, homocysteine, homoserine, ornithine and methionine sulfone. Additional types of prodrugs are also encompassed. For instance, free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem., 1996, 29, 10. Free amines can also be derivatized as amides, sulfonamides or phosphonamides. All of these prodrug moieties may incorporate groups including but not limited to ether, amine and carboxylic acid functionalities.

The compounds of the present invention can be used in the methods of the invention in combination with other drugs. For example, dosing a SARS-CoV-2 coronavirus-infected patient (i.e., a patient with COVID-19) with the SARS-CoV-2 coronavirus 3CL protease inhibitor of the invention and an interferon, such as interferon alpha, or a pegylated interferon, such as PEG-Intron or Pegasus, may provide a greater clinical benefit than dosing either the interferon, pegylated interferon or the SARS-CoV-2 coronavirus inhibitor alone. Other additional agents that can be used in the methods of the present invention include dexamethasone, azithromycin and remdesivir. Examples of greater clinical benefits could include a larger reduction in COVID-19 symptoms, a faster time to alleviation of symptoms, reduced lung pathology, a larger reduction in the amount of SARS-CoV-2 coronavirus in the patient (viral load), and decreased mortality.

The SARS-CoV-2 coronavirus infects cells which express P-glycoprotein. Some of the SARS-CoV-2 coronavirus 3CL protease inhibitors of the invention are P-glycoprotein substrates. Compounds which inhibit the SARS-CoV-2 coronavirus which are also P-glycoprotein substrates may be dosed with a P-glycoprotein inhibitor. Examples of P-glycoprotein inhibitors are verapamil, vinblastine, ketoconazole, nelfinavir, ritonavir or cyclosporine. The P-glycoprotein inhibitors act by inhibiting the efflux of the SARS-CoV-2 coronavirus inhibitors of the invention out of the cell. The inhibition of the P-glycoprotein-based efflux will prevent reduction of intracellular concentrations of the SARS-CoV-2 coronavirus inhibitor due to P-glycoprotein efflux. Inhibition of the P-glycoprotein efflux will result in larger intracellular concentrations of the SARS-CoV-2 coronavirus inhibitors. Dosing a SARS-CoV-2 coronavirus-infected patient with the SARS-CoV-2 coronavirus 3CL protease inhibitors of the invention and a P-glycoprotein inhibitor may lower the amount of SARS-CoV-2 coronavirus 3CL protease inhibitor required to achieve an efficacious dose by increasing the intracellular concentration of the SARS-CoV-2 coronavirus 3CL protease inhibitor.

Among the agents that may be used to increase the exposure of a mammal to a compound of the present invention are those that can act as inhibitors of at least one isoform of the cytochrome P450 (CYP450) enzymes. The isoforms of CYP450 that may be beneficially inhibited include, but are not limited to CYP1A2, CYP2D6, CYP2C9, CYP2C19 and CYP3A4. The compounds used in the methods of the invention include compounds that may be CYP3A4 substrates and are metabolized by CYP3A4. Dosing a SARS-CoV-2 coronavirus-infected patient with a SARS-CoV-2 coronavirus inhibitor which is a CYP3A4 substrate, such as SARS-CoV-2 coronavirus 3CL protease inhibitor, and a CYP3A4 inhibitor, such as ritonavir, nelfinavir or delavirdine, will reduce the metabolism of the SARS-CoV-2 coronavirus inhibitor by CYP3A4. This will result in reduced clearance of the SARS-CoV-2 coronavirus inhibitor and increased SARS-CoV-2 coronavirus inhibitor plasma concentrations. The reduced clearance and higher plasma concentrations may result in a lower efficacious dose of the SARS-CoV-2 coronavirus inhibitor.

Additional therapeutic agents that can be used in combination with the SARS-CoV-2 inhibitors in the methods of the present invention include the following:

PLpro inhibitors, Apilomod, EIDD-2801, Ribavirin, Valganciclovir, #-Thymidine, Aspartame, Oxprenolol, Doxycycline, Acetophenazine, Iopromide, Riboflavin, Reproterol, 2,2′-Cyclocytidine, Chloramphenicol, Chlorphenesin carbamate, Levodropropizine, Cefamandole, Floxuridine, Tigecycline, Pemetrexed, L(+)-Ascorbic acid, Glutathione, Hesperetin, Ademetionine, Masoprocol, Isotretinoin, Dantrolene, Sulfasalazine Anti-bacterial, Silybin, Nicardipine, Sildenafil, Platycodin, Chrysin, Neohesperidin, Baicalin, Sugetriol-3,9-diacetate, (−)-Epigallocatechin gallate, Phaitanthrin D, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, 2,2-di(3-indolyl)-3-indolone, (S)-(1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl-2-amino-3-phenylpropanoate, Piceatannol, Rosmarinic acid, and Magnolol.

3CLpro inhibitors, Lymecycline, Chlorhexidine, Alfuzosin, Cilastatin, Famotidine, Almitrine, Progabide, Nepafenac, Carvedilol, Amprenavir, Tigecycline, Montelukast, Carminic acid, Mimosine, Flavin, Lutein, Cefpiramide, Phenethicillin, Candoxatril, Nicardipine, Estradiol valerate, Pioglitazone, Conivaptan, Telmisartan, Doxycycline, Oxytetracycline, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl) pentanoate, Betulonal, Chrysin-7-O-β-glucuronide, Andrographiside, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 2-nitrobenzoate, 2β-Hydroxy-3,4-seco-friedelolactone-27-oic acid (S)-(1S,2R,4aS,5R,8aS)-1-Formamido-1,4a-dimethyl-6-methylene-5-((E)-2-(2-oxo-2,5-dihydrofuran-3-yl)ethenyl) decahydronaphthalen-2-yl-2-amino-3-phenylpropanoate, Isodecortinol, Cerevisterol, Hesperidin, Neohesperidin, Andrograpanin, 2-((1R,5R,6R,8aS)-6-Hydroxy-5-(hydroxymethyl)-5,8a-dimethyl-2-methylenedecahydronaphthalen-1-yl)ethyl benzoate, Cosmosiin, Cleistocaltone A, 2,2-Di(3-indolyl)-3-indolone, Biorobin, Gnidicin, Phyllaemblinol, Theaflavin 3,3′-di-O-gallate, Rosmarinic acid, Kouitchenside I, Oleanolic acid, Stigmast-5-en-3-ol, Deacetylcentapicrin, and Berchemol.

RdRp inhibitors, Valganciclovir, Chlorhexidine, Ceftibuten, Fenoterol, Fludarabine, Itraconazole, Cefuroxime, Atovaquone, Chenodeoxycholic acid, Cromolyn, Pancuronium bromide, Cortisone, Tibolone, Novobiocin, Silybin, Idarubicin Bromocriptine, Diphenoxylate, Benzylpenicilloyl G, Dabigatran etexilate, Betulonal, Gnidicin, 2β,30β-Dihydroxy-3,4-seco-friedelolactone-27-lactone, 14-Deoxy-11,12-didehydroandrographolide, Gniditrin, Theaflavin 3,3′-di-O-gallate, (R)-((1R,5aS,6R,9aS)-1,5a-Dimethyl-7-methylene-3-oxo-6-((E)-2-(2-oxo-2,5-dihydrofuran-3-yl)ethenyl)decahydro-1H-benzo[c]azepin-1-yl)methyl 2-amino-3-phenylpropanoate, 2β-Hydroxy-3,4-seco-friedelolactone-27-oic acid, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, Phyllaemblicin B, 14-hydroxycyperotundone, Andrographiside, 2-((1R,5R,6R,8aS)-6-Hydroxy-5-(hydroxymethyl)-5,8a-dimethyl-2-methylenedecahydro naphthalen-1-yl)ethyl benzoate, Andrographolide, Sugetriol-3,9-diacetate, Baicalin, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl)pentanoate, 1,7-Dihydroxy-3-methoxyxanthone, 1,2,6-Trimethoxy-8-[(6-O-β-D-xylopyranosyl-β-D-glucopyranosyl)oxy]-9H-xanthen-9-one, and 1,8-Dihydroxy-6-methoxy-2-[(6-O-β-D-xylopyranosyl-β-D-glucopyranosyl)oxy]-9H-xanthen-9-one, 8-(β-D-Glucopyranosyloxy)-1,3,5-trihydroxy-9H-xanthen-9-one,

Additional therapeutic agents that can be used in the methods of the invention include Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, Lopinavir/Ritonavir+Ribavirin, Alferon, and prednisone. Other additional agents useful in the methods of the present invention include dexamethasone, azithromycin and remdesivir as well as boceprevir, umifenovir and favipiravir.

Other additional agents that can be used in the methods of the present invention include α-ketoamides compounds designated as 11r, 13a and 13b, shown below, as described in Zhang, L.; Lin, D.; Sun, X.; Rox, K.; Hilgenfeld, R.; X-ray Structure of Main Protease of the Novel Coronavirus SARS-CoV-2 Enables Design of α-Ketoamide Inhibitors; bioRxiv preprint doi: https://doi.org/10.1101/2020.02.17.952879

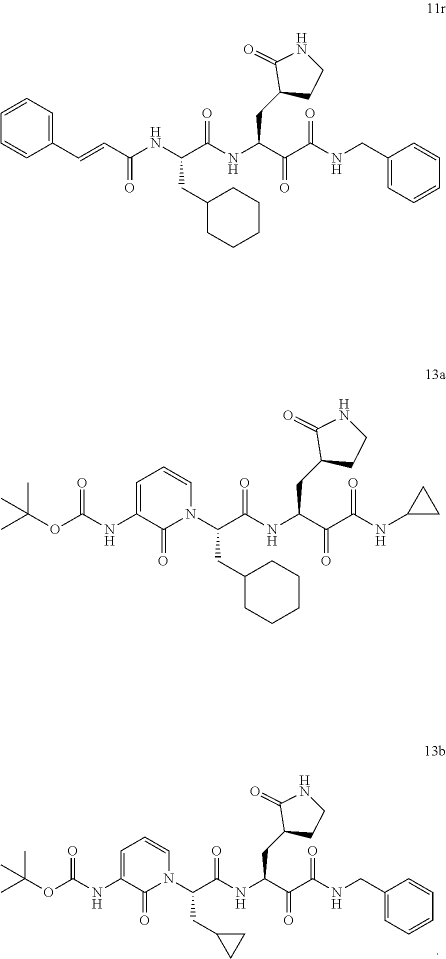 [SMILES: O=C(/C=C/c1ccccc1)N[C@@H](CC1CCCCC1)C(=O)N[C@@H](C[C@@H]1CCNC1=O)C(=O)C(=O)NCc1ccccc1 ; CC(C)(C)OC(=O)Nc1cccn([C@@H](CC2CCCCC2)C(=O)N[C@@H](C[C@@H]2CCNC2=O)C(=O)C(=O)NC2CC2)c1=O ; CC(C)(C)OC(=O)Nc1cccn([C@@H](CC2CC2)C(=O)N[C@@H](C[C@@H]2CCNC2=O)C(=O)C(=O)NCc2ccccc2)c1=O]

Additional agents that can be used in the methods of the present invention include RIG 1 pathway activators such as those described in U.S. Pat. No. 9,884,876.

Other additional therapeutic agents include protease inhibitors such as those described in Dai W, Zhang B, Jiang X-M, et al Structure-based design of antiviral drug candidates targeting the SARS-CoV-2 main protease. Science. 2020; 368(6497):1331-1335 including compounds such as the compound shown below and a compound designated as DC402234

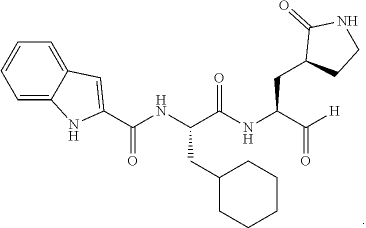 [SMILES: O=C[C@H](C[C@@H]1CCNC1=O)NC(=O)[C@H](CC1CCCCC1)NC(=O)c1cc2ccccc2[nH]1]

Another embodiment of the present invention is a method of treating COVID-19 in a patient wherein in addition to administering a compound of the present invention (i.e. a compound of Formula I or a pharmaceutically acceptable salt thereof) an additional agent is administered and the additional agent is selected from antivirals such as remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir and ABX464, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and sarilumab (Kevzara), PlKfyve inhibitors such as apilimod dimesylate, RIPK1 inhibitors such as DNL758, VIP receptor agonists such as PB1046, SGLT2 inhibitors such as dapaglifozin, TYK inhibitors such as abivertinib, kinase inhibitors such as ATR-002, bemcentinib, acalabrutinib and losmapimod, H2 blockers such as famotidine, anthelmintics such as niclosamide, furin inhibitors such as diminazene, kinase inhibitors baricitinib and tofacitinib and protease inhibitors such as DC402234.

The term “SARS-CoV-2 inhibiting agent” means any SARS-CoV-2-related coronavirus 3C-like protease inhibitor compound described herein or a pharmaceutically acceptable salt, hydrate, prodrug, active metabolite or solvate thereof or a compound which inhibits replication of SARS-CoV-2 in any manner.

The term “interfering with or preventing” SARS-CoV-2-related coronavirus (“SARS-CoV-2”) viral replication in a cell means to reduce SARS-CoV-2 replication or production of SARS-CoV-2 components necessary for progeny virus in a cell treated with a compound of this invention as compared to a cell not being treated with a compound of this invention. Simple and convenient assays to determine if SARS-CoV-2 viral replication has been reduced include an ELISA assay for the presence, absence, or reduced presence of anti-SARS-CoV-2 antibodies in the blood of the subject (Nasoff, et al., PNAS 88:5462-5466, 1991), RT-PCR (Yu, et al., in Viral Hepatitis and Liver Disease 574-577, Nishioka, Suzuki and Mishiro (Eds.); Springer-Verlag, Tokyo, 1994). Such methods are well known to those of ordinary skill in the art. Alternatively, total RNA from transduced and infected “control” cells can be isolated and subjected to analysis by dot blot or northern blot and probed with SARS-CoV-2-specific DNA to determine if SARS-CoV-2 replication is reduced. Alternatively, reduction of SARS-CoV-2 protein expression can also be used as an indicator of inhibition of SARS-CoV-2 replication. A greater than fifty percent reduction in SARS-CoV-2 replication as compared to control cells typically quantitates a prevention of SARS-CoV-2 replication.

If a SARS-CoV-2 inhibitor compound used in the method of the invention is a base, a desired salt may be prepared by any suitable method known to the art, including treatment of the free base with an inorganic acid (such as hydrochloric acid, hydrobromic acid, sulfuric acid, nitric acid, phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like.

If a SARS-CoV-2 inhibitor compound used in the method of the invention is an acid, a desired salt may be prepared by any suitable method known to the art, including treatment of the free acid with an inorganic or organic base [such as an amine (primary, secondary, or tertiary)], an alkali metal hydroxide, or alkaline earth metal hydroxide. Illustrative examples of suitable salts include organic salts derived from amino acids (such as glycine and arginine), ammonia, primary amines, secondary amines, tertiary amines, and cyclic amines (such as piperidine, morpholine, and piperazine), as well as inorganic salts derived from sodium, calcium, potassium, magnesium, manganese, iron, copper, zinc, aluminum and lithium.

In the case of SARS-CoV-2 inhibitor compounds, prodrugs, salts, or solvates that are solids, it is understood by those skilled in the art that the compound, prodrugs, salts, and solvates used in the method of the invention, may exist in different polymorph or crystal forms, all of which are intended to be within the scope of the present invention and specified formulas. In addition, the compound, salts, prodrugs and solvates used in the method of the invention may exist as tautomers, all of which are intended to be within the broad scope of the present invention.

Solubilizing agents may also be used with the compounds of the invention to increase the compounds' solubility in water of physiologically acceptable solutions. These solubilizing agents include cyclodextrins, propylene glycol, diethylacetamide, polyethylene glycol, Tween, ethanol and micelle-forming agents. Offered solubilizing agents are cyclodextrins, particularly beta-cyclodextrins and in particular hydroxypropyl beta-cyclodextrin and sulfobutylether beta-cyclodextrin.

In some cases, the SARS-CoV-2 inhibitor compounds, salts, prodrugs and solvates used in the method of the invention may have chiral centers. When chiral centers are present, the compound, salts, prodrugs and solvates may exist as single stereoisomers, racemates, and/or mixtures of enantiomers and/or diastereomers. All such single stereoisomers, racemates, and mixtures thereof are intended to be within the broad scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacologically pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

The term “treating”, as used herein, unless otherwise indicated, means reversing, alleviating, inhibiting the progress of, or preventing the disorder of condition to which such term applies, or one or more symptoms of such disorder or condition. In the methods of treating COVID-19 it is to be understood that COVID-19 is the disease caused in patients by infection with the SARS-CoV-2 virus. The SARS-CoV-2 virus is to be understood to encompass the initially discovered strain of the virus as well as mutant strains which emerge, such as but not limited to, strains such as B.1.1.7 (UK variant), B.1.351 (South African variant), P.1 (Brazilian variant) and B.1.427 and B.1.429 (California variants). The term “treatment”, as used herein, unless otherwise indicated, refers to the act of treating as “treating” is defined immediately above.

In a preferred embodiment of the present invention, “treating” or “treatment” means at least the mitigation of a disease condition in a human, that is alleviated by the inhibition of the activity of the SARS-CoV-2 3C-like protease which is the main protease of SARS-CoV-2, the causative agent for COVID-19. For patients suffering from COVID-19, fever, fatigue, and dry cough are the main manifestations of the disease, while nasal congestion, runny nose, and other symptoms of the upper respiratory tract are rare. Beijing Centers for Diseases Control and Prevention indicated that the typical case of COVID-19 has a progressive aggravation process. COVID-19 can be classified into light, normal, severe, and critical types based on the severity of the disease. National Health Commission of the People's Republic of China. Diagnosis and Treatment of Pneumonia Caused by 2019-nCoV (Trial Version 4). Available online: http://www.nhc.gov.cn/jkj/s3577/202002/573340613ab243b3a7f61df260551dd4/files/c791e5a7ea5149f680fdcb34dacOf54e.pdf: (1) Mild cases—the clinical symptoms were mild, and no pneumonia was found on the chest computed tomography (CT); (2) normal cases-fever, respiratory symptoms, and patients found to have imaging manifestations of pneumonia; (3) severe cases—one of the following three conditions: Respiratory distress, respiratory rate ≥30 times/min (in resting state, refers to oxygen saturation s 93%), partial arterial oxygen pressure (PaO2)/oxygen absorption concentration (FiO2) ≤300 mmHg (1 mm Hg=0.133 kPa); (4) critical cases—one of the following three conditions: Respiratory failure and the need for mechanical ventilation, shock, or the associated failure of other organs requiring the intensive care unit. The current clinical data shows that the majority of deaths occurred in the older patients. However, severe cases have been documented in young adults who have unique factors, particularly those with chronic diseases, such as diabetes or hepatitis B. Those with a long-term use of hormones or immunosuppressants, and decreased immune function, are likely to get severely infected.

Methods of treatment for mitigation of a coronavirus disease condition such as COVID-19 include the use of one or more of the compounds of the invention in any conventionally acceptable manner. According to certain preferred embodiments of the invention, the compound or compounds used in the methods of the present invention are administered to a mammal, such as a human, in need thereof. Preferably, the mammal in need thereof is infected with a coronavirus such as the causative agent of COVID-19, namely SARS-CoV-2.

The present invention also includes prophylactic methods, comprising administering an effective amount of a SARS-CoV-2 inhibitor of the invention, or a pharmaceutically acceptable salt, prodrug, pharmaceutically active metabolite, or solvate thereof to a mammal, such as a human at risk for infection by SARS-CoV-2. According to certain preferred embodiments, an effective amount of one or more compounds of the invention, or a pharmaceutically acceptable salt, prodrug, pharmaceutically active metabolite, or solvate thereof is administered to a human at risk for infection by SARS-CoV-2, the causative agent for COVID-19. The prophylactic methods of the invention include the use of one or more of the compounds in the invention in any conventionally acceptable manner.

Certain of the compounds used in the methods of the invention, for example dexamethasone, azithromycin and remdesivir are known and can be made by methods known in the art.

Recent evidence indicates that a new coronavirus SARS-CoV-2 is the causative agent of COVID-19. The nucleotide sequence of the SARS-CoV-2 coronavirus as well as the recently determined L- and S-subtypes have recently been determined and made publicly available.

The activity of the inhibitor compounds as inhibitors of SARS-CoV-2 viral activity may be measured by any of the suitable methods available in the art, including in vivo and in vitro assays. The activity of the compounds of the present invention as inhibitors of coronavirus 3C-like protease activity (such as the 3C-like protease of the SARS-CoV-2 coronavirus) may be measured by any of the suitable methods known to those skilled in the art, including in vivo and in vitro assays. Examples of suitable assays for activity measurements include the antiviral cell culture assays described herein as well as the antiprotease assays described herein, such as the assays described in the Experimental section.

Administration of the SARS-CoV-2 inhibitor compounds and their pharmaceutically acceptable prodrugs, salts, active metabolites, and solvates may be performed according to any of the accepted modes of administration available to those skilled in the art. Illustrative examples of suitable modes of administration include oral, nasal, pulmonary, parenteral, topical, intravenous, injected, transdermal, and rectal. Oral, intravenous, subcutaneous and nasal deliveries are preferred.

A SARS-CoV-2-inhibiting agent may be administered as a pharmaceutical composition in any suitable pharmaceutical form. Suitable pharmaceutical forms include solid, semisolid, liquid, or lyophilized formulations, such as tablets, powders, capsules, suppositories, suspensions, liposomes, and aerosols. The SARS-CoV-2-inhibiting agent may be prepared as a solution using any of a variety of methodologies. For example, SARS-CoV-2-inhibiting agent can be dissolved with acid (e.g., 1 M HCl) and diluted with a sufficient volume of a solution of 5% dextrose in water (D5W) to yield the desired final concentration of SARS-CoV-2-inhibiting agent (e.g., about 15 mM). Alternatively, a solution of D5W containing about 15 mM HCl can be used to provide a solution of the SARS-CoV-2-inhibiting agent at the appropriate concentration. Further, the SARS-CoV-2-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling and dissolving the ingredients as appropriate, to give the desired products for intravenous, oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of a SARS-CoV-2-inhibiting agent and preferably is made up of one or more pharmaceutical dosage units. The selected dose may be administered to a mammal, for example, a human patient, in need of treatment mediated by inhibition of SARS-CoV-2 related coronavirus activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion.

The phrases “therapeutically effective amount” and “effective amount” are intended to mean the amount of an inventive agent that, when administered to a mammal in need of treatment, is sufficient to effect treatment for injury or disease conditions alleviated by the inhibition of SARS-CoV-2 viral replication. The amount of a given SARS-CoV-2-inhibiting agent used in the method of the invention that will be therapeutically effective will vary depending upon factors such as the particular SARS-CoV-2-inhibiting agent, the disease condition and the severity thereof, the identity and characteristics of the mammal in need thereof, which amount may be routinely determined by those skilled in the art.

It will be appreciated that the actual dosages of the SARS-CoV-2-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.01 to about 1000 mg/kg body weight, preferably from about 0.1 to about 500 mg/kg body weight, and even more preferably from about 1 to about 500 mg/kg body weight, with courses of treatment repeated at appropriate intervals. For intravenous dosing a dose of up to 5 grams per day may be employed. Intravenous administration can occur for intermittent periods during a day or continuously over a 24-hour period.

The terms “cytochrome P450-inhibiting amount” and “cytochrome P450 enzyme activity-inhibiting amount”, as used herein, refer to an amount of a compound required to decrease the activity of cytochrome P450 enzymes or a particular cytochrome P450 enzyme isoform in the presence of such compound. Whether a particular compound decreases cytochrome P450 enzyme activity, and the amount of such a compound required to do so, can be determined by methods know to those of ordinary skill in the art and the methods described herein.

Protein functions required for coronavirus replication and transcription are encoded by the so-called “replicase” gene. Two overlapping polyproteins are translated from this gene and extensively processed by viral proteases. The C-proximal region is processed at eleven conserved interdomain junctions by the coronavirus main or “3C-like” protease. The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. A comparison of the amino acid sequence of the SARS-CoV-2-associated coronavirus 3C-like protease to that of other known coronaviruses such as SARS-CoV shows the amino acid sequences have approximately 96% shared homology.

Amino acids of the substrate in the protease cleavage site are numbered from the N to the C terminus as follows: -P3-P2-P1-P1′-P2′-P3′, with cleavage occurring between the P1 and P1′ residues (Schechter & Berger, 1967). Substrate specificity is largely determined by the P2, P1 and P1′ positions. Coronavirus main protease cleavage site specificities are highly conserved with a requirement for glutamine at P1 and a small amino acid at P1′ [Journal of General Virology, 83, pp. 595-599 (2002)].

The compounds of the present invention can be prepared according to the methods set forth in Reaction Schemes 1 to 4 below.

The schemes provided below further illustrate and exemplify the compounds of the present invention and methods of preparing such compounds. It is to be understood that the scope of the present invention is not limited in any way by the scope of the following examples and preparations. In the following examples molecules with a single chiral center may exist as a single enantiomer or a racemic mixture. Those molecules with two or more chiral centers may exist as a single enantiomer, a racemic or otherwise mixture of two enantiomers, or as various mixtures of diastereomers. Such enantiomers, racemates, and diastereomers may be obtained and/or separated by methods known to those skilled in the art. It will be appreciated by one skilled in the art that certain synthetic manipulations may epimerize or racemize a stereocenter, and synthetic conditions may be selected to either promote or discourage such epimerization or racemization.

Scheme 1 illustrates a synthetic sequence for the preparation of compounds of Formula I as shown, wherein the definitions of R¹, R², R³, Ring A, p, q and q′ are as defined herein. The compound of Formula 1, wherein PG is an appropriate amine protecting group, can be treated with excess base such as lithium diisopropylamide and chloroiodomethane in a suitable solvent like tetrahydrofuran (THF) to afford the chloro compound of Formula 2 (see for example Hoffman, R., et al., Journal of Medicinal Chemistry, 63, 2020, 12725-12747).

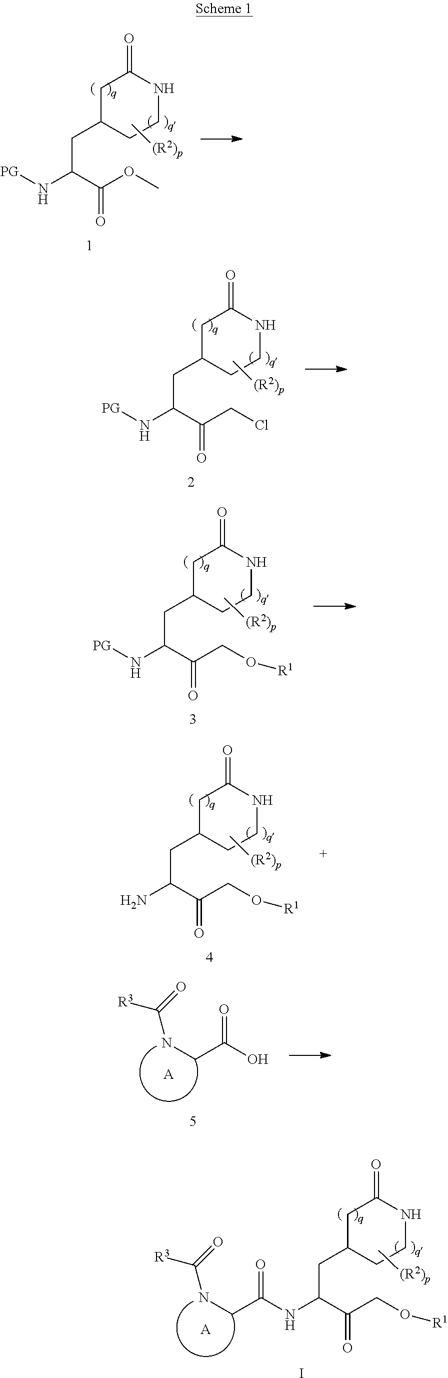

The chloro compound 2 may be converted into compounds of the Formula 3 directly by reaction with a suitable alcohol of formula R¹OH, including especially phenols and heteroaryl alcohols, in the presence of a variety of bases. Such bases include but are not limited to cesium fluoride, potassium hydroxide and especially carbonate bases such as sodium and potassium carbonate. Suitable solvents include, but are not limited to, dichloromethane (CH₂Cl₂), N,N-dimethylformamide (DMF), toluene, and especially THF. The compound of Formula 3 may be N-deprotected to provide an amine of Formula 4 using methods well known to those skilled in the art for effecting such deprotections. Frequently acidic reagents such as hydrogen chloride, methanesulfonic acid, or trifluoroacetic acid are used, typically in a reaction-compatible solvent such as CH₂Cl₂, 1,4-dioxane, ethyl acetate (EtOAc), or acetonitrile (CH₃CN). One skilled in the art will appreciate that the compound of Formula 4 will frequently be obtained as an acid addition salt. The compound of Formula 4 may then be transformed into a compound of Formula I by treatment with a compound of Formula 5 under appropriate conditions. For example, the compound of Formula 5 may be treated with a reagent such as O-(7-azabenzotriazol-1-yl)-N,N,N′,N′-tetramethyluronium hexafluorophosphate (HATU), isobutyl chloroformate, 1-[3-(dimethylamino)propyl]-3-ethylcarbodiimide hydrochloride (EDCl) and hydroxybenzotriazole (HOBt), or 1,1′-carbonyldiimidazole (CDI), optionally in the presence of a base such as N,N-diisopropylethylamine (DIEA), 4-methylmorpholine (NMM), or triethylamine (TEA), followed by treatment with a compound of the Formula 4 in the presence of a base such as N,N-diisopropylethylamine, 4-methylmorpholine, or triethylamine. Suitable solvents include, but are not limited to, CH₂Cl₂, EtOAc, DMF, THF, or CH₃CN. Compounds of Formula 5 are exceptionally well known in the chemical literature, and one skilled in the art may choose to prepare any given compound of Formula 5 using methods analogous to those described in the chemical literature

Scheme 1A illustrates the same synthetic sequence as in Scheme 1 for the preparation of compounds of Formula I′ as shown, wherein the amine protecting group is tert-butoxycarbonyl (Boc) and the lactam ring is the 5-membered oxopyrrolidine ring as depicted. The compound of Formula 1′ (WO2005/11580) can be treated with excess base such as lithium diisopropylamide and chloroiodomethane in a suitable solvent like tetrahydrofuran (THF) to afford the compound of Formula 2′ (Hoffman, R., et al., Journal of Medicinal Chemistry, 63, 2020, 12725-12747).

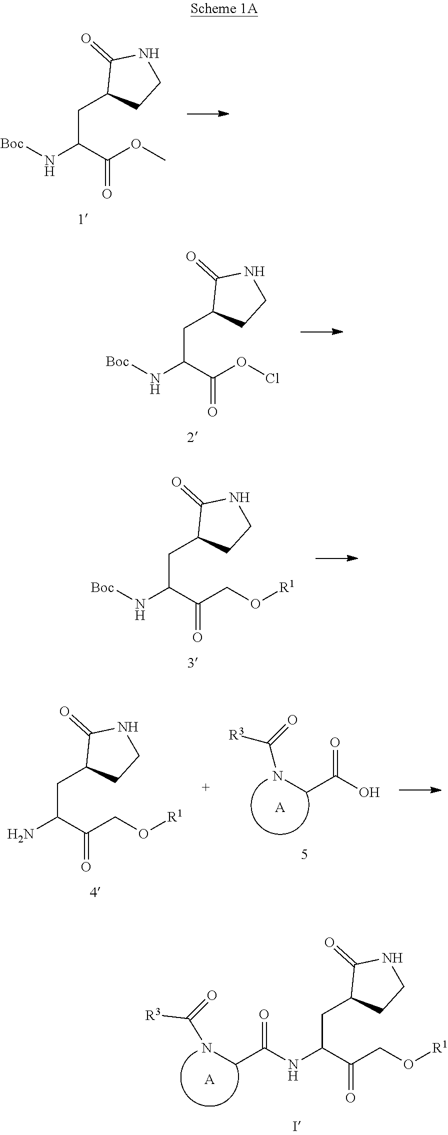

Compound 2′ may be converted into compounds of the Formula 3′ directly by reaction with a suitable alcohol, including especially phenols and heteroaryl alcohols, in the presence of a variety of bases. Such bases include but are not limited to cesium fluoride, potassium hydroxide and especially carbonate bases such as sodium and potassium carbonate. Suitable solvents include, but are not limited to, dichloromethane (CH₂Cl₂), N,N-dimethylformamide (DMF), toluene, and especially THF. The compound of Formula 3′ may be N-deprotected to provide an amine of Formula 4′ using methods well known to those skilled in the art for effecting such deprotections. Frequently acidic reagents such as hydrogen chloride, methanesulfonic acid, or trifluoroacetic acid are used, typically in a reaction-compatible solvent such as CH₂Cl₂, 1,4-dioxane, ethyl acetate (EtOAc), or acetonitrile (CH₃CN). One skilled in the art will appreciate that the compound of Formula 4′ will frequently be obtained as an acid addition salt. The compound of Formula 4′ may then be transformed into a compound of Formula I′ by treatment with a compound of Formula 5 under appropriate conditions. For example, the compound of Formula 5 may be treated with a reagent such as 0-(7-azabenzotriazol-1-yl)-N,N,N′,N′-tetramethyluronium hexafluorophosphate (HATU), isobutyl chloroformate, 1-[3-(dimethylamino)propyl]-3-ethylcarbodiimide hydrochloride (EDCl) and hydroxybenzotriazole (HOBt), or 1,1′-carbonyldiimidazole (CDI), optionally in the presence of a base such as N,N-diisopropylethylamine (DIEA), 4-methylmorpholine (NMM), or triethylamine (TEA), followed by treatment with a compound of the Formula 4′ in the presence of a base such as N,N-diisopropylethylamine, 4-methylmorpholine, or triethylamine. Suitable solvents include, but are not limited to, CH₂Cl₂, EtOAc, DMF, THF, or CH₃CN. Compounds of Formula 5 are exceptionally well known in the chemical literature, and one skilled in the art may choose to prepare any given compound of Formula 5 using methods analogous to those described in the chemical literature.

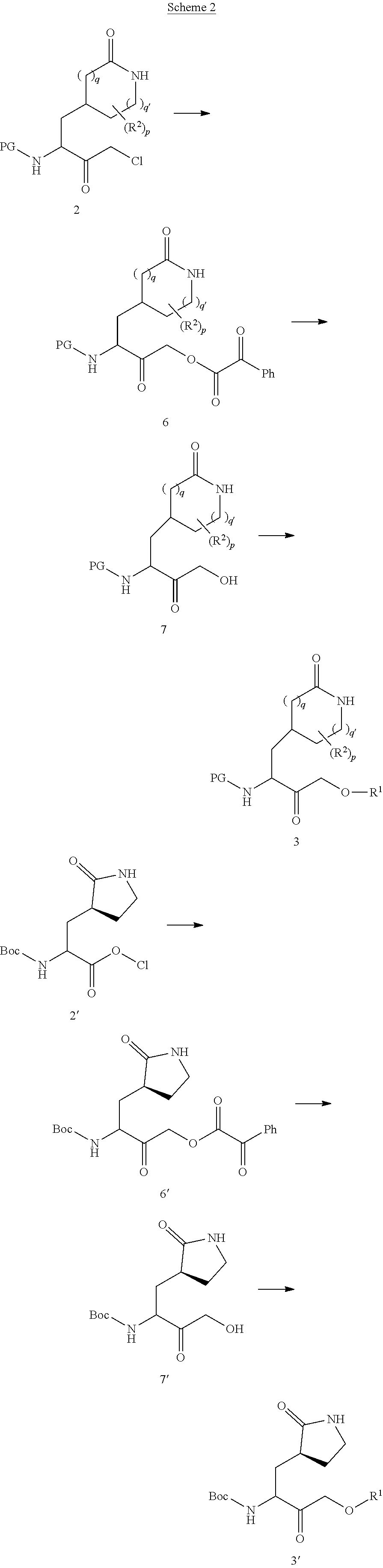

Scheme 2 provides an alternative method for preparing the intermediates of Formula 3 and Formula 3′ as described above in Schemes 1 and 1A. In Scheme 2 the variables p, q, q′ and R² are as described herein, PG is an appropriate amine protecting group and Boc is tert-butoxy carbonyl. The compounds 2 or 2′ may be used to prepare the compound of Formula 6 or 6′, respectively, from phenyl glyoxalic acid by treatment with an appropriate base such as cesium fluoride, in a suitable solvent such as DMF (for general procedure see Hoffman, R., et al., Journal of Medicinal Chemistry, 63, 2020, 12725-12747). Compound 7 or 7′ can in turn be obtained by selective deprotection of the corresponding compound 6 or 6′ ester under a variety of conditions including treatment with potassium carbonate in a suitable solvent such as methanol. Alkylation of the compounds of Formula 7 or 7′ with an appropriate alkyl halide, R¹X wherein X is a halide, such as methyl iodide or a benzyl halide, in the presence of an appropriate base such as silver oxide, in a suitable solvent such as 1,2-dichloroethane (DCE) or especially CH₃CN, also allows preparation of compounds of Formula 3 or 3′, respectively. Other alkylating agents, including trichloroacetimidate reagents such as 4-methoxybenzyl trichloroacetimidate, may also effect this transformation in an appropriate solvent, such as CH₂Cl₂. Compounds of Formula 3 or 3′ may also be obtained from compounds of Formula 7 or 7′, respectively, by treatment with an appropriate alcohol, including phenols and fluorinated alcohols like hexafluoroisopropanol, and a reagent such as (cyanomethylene)tributylphosphorane or reagent combinations such as triphenylphosphine (optionally polymer-supported) and diisopropyl azodicarboxylate (DIAD) in a suitable solvent such as toluene or THF. Compounds of Formula 3 or 3′ may be further elaborated as previously described to provide compounds of Formula I and I′, respectively.

One skilled in the art will appreciate that the bond-forming steps above may be conducted in a different order with appropriate considerations, for example as shown below in Scheme 3.

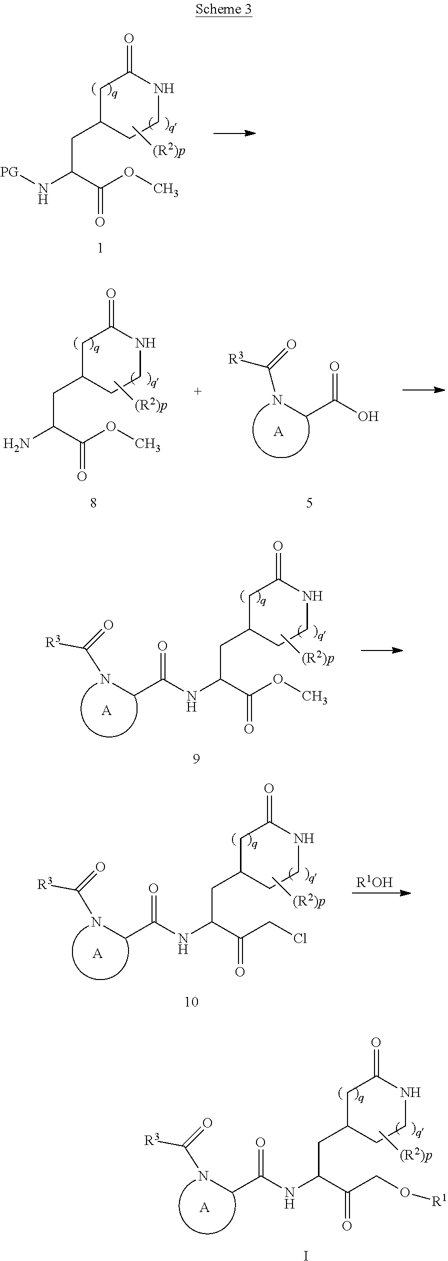

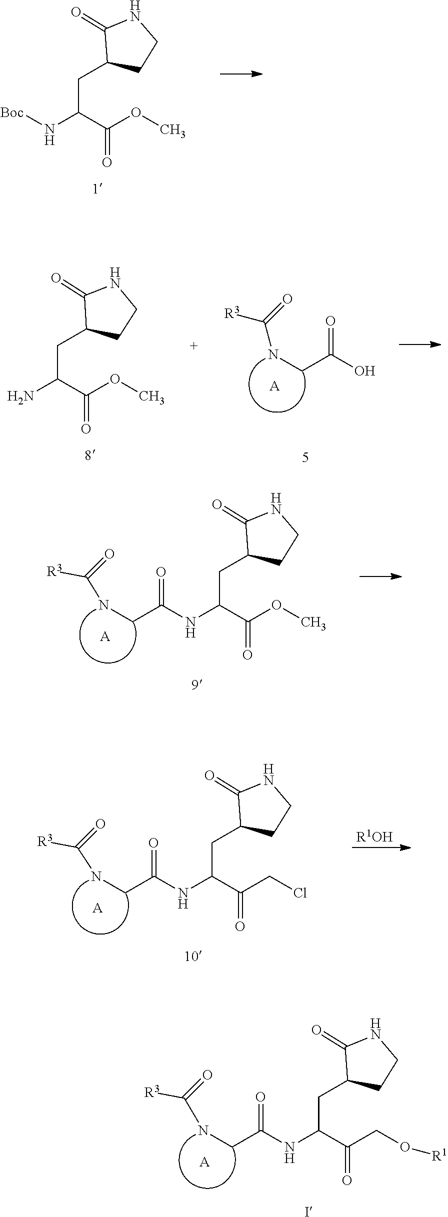

The compound of Formula 1 or 1′ may be N-deprotected by removal of the appropriate amine protecting group PG or tert-butoxycarbonyl (Boc) group, respectively, to provide an amine of Formula 8 or 8′ using methods well known to those skilled in the art for effecting such deprotections. Frequently acidic reagents such as hydrogen chloride, methanesulfonic acid, or trifluoroacetic acid are used, typically in a reaction-compatible solvent such as CH₂Cl₂, 1,4-dioxane, EtOAc, or CH₃CN. One skilled in the art will appreciate that the compound of Formula 8 or 8′ will frequently be obtained as an acid addition salt. The compound of Formula 8 or 8′ may then be transformed into a compound of Formula 9 or 9′, respectively, by treatment with a compound of Formula 5 under appropriate conditions. Such methods are well known to those skilled in the art, and in general standard peptide coupling conditions may be selected. Subsequently, a compound of Formula 9 or 9′ can be further elaborated to a corresponding chloromethylketone compound of Formula 10 or 10′ by treatment with chloroacetic acid, or suitable salt thereof such as sodium chloroacetate, in the presence of an excess of a strong base such as tert-butyl magnesium chloride, with an appropriate tertiary amine base, such as trimethylamine or DIEA, in a suitable solvent such as THF. The compound of Formula 10 or 10′ can be transformed into a corresponding compound of Formula I or I′ by alkylation of a suitable alcohol, including especially phenols and heteroaryl alcohols, in the presence of a variety of bases. Such bases include but are not limited to cesium fluoride, potassium hydroxide and especially carbonate bases such as sodium and potassium carbonate. Suitable solvents include, but are not limited to CH₂Cl₂, DMF, toluene, and especially THF.

Scheme 4 provides a further example that the bond-forming steps above may be conducted in a different order with appropriate considerations.

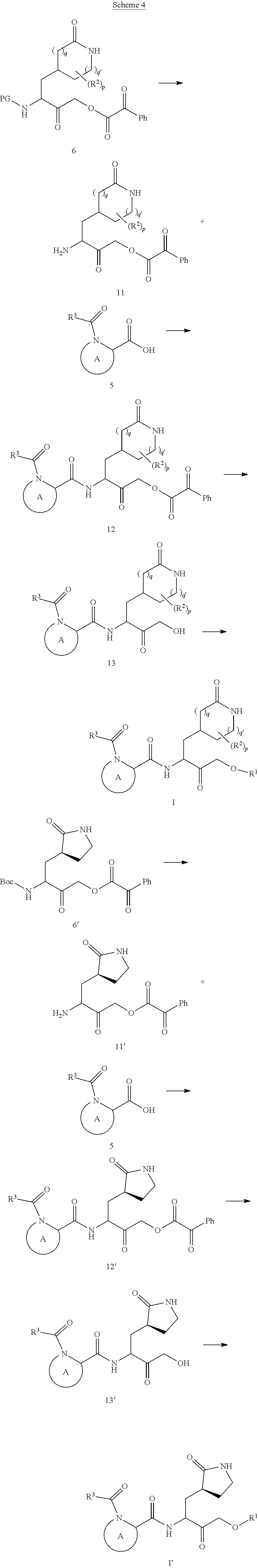

The compound of Formula 6 or 6′ may be N-deprotected to provide an amine of Formula 11 or 11′ using methods well known to those skilled in the art for effecting such deprotections. Frequently acidic reagents such as hydrogen chloride, methanesulfonic acid, or trifluoroacetic acid are used, typically in a reaction-compatible solvent such as CH₂Cl₂, 1,4-dioxane, EtOAc, or CH₃CN. One skilled in the art will appreciate that the compound of Formula 11 or 11′ will frequently be obtained as an acid addition salt. The compound of Formula 11 or 11′ may then be transformed into a compound of Formula 12 or 12′ by treatment with a carboxylic acid compound of Formula 5 under standard coupling conditions. One skilled in the art may choose to use, for example, a carbodiimide reagent such as 1-[3-(dimethylamino)propyl]-3-ethylcarbodiimide hydrochloride (EDCl) or N,N′-dicyclohexyl carbodiimide (DCC), optionally in the presence of an auxiliary nucleophile such as hydroxybenzotriazole (HOBt) or 2-hydroxypyridine-N-oxide (HOPO), frequently in the presence of a base such as TEA or DIEA. Suitable solvents include, but are not limited to, CH₂C12, EtOAc, THF, or CH₃CN. Once formed, a compound of Formula 12 or 12′ may be deprotected to afford the hydroxyl compound of Formula 13 or 13′ using a variety of conditions including treatment with potassium carbonate in a suitable solvent such as methanol. Alkylation of compounds of Formula 13 or 13′ with an appropriate alkyl halide, such as methyl iodide or a benzyl halide, in the presence of an appropriate base such as silver oxide in a suitable solvent such as 1,2-dichloroethane (DCE) or especially CH₃CN, also allows preparation of compounds of Formula I or I′. Other alkylating agents, such as 4-methoxybenzyl trichloroacetimidate, may also effect this transformation in an appropriate solvent, such as CH₂Cl₂. Compounds of Formula I may also be obtained from compounds of formula 13 or 13′ by treatment with an appropriate alcohol, including phenols and fluorinated alcohols like hexafluoroisopropanol, and a reagent such as (cyanomethylene)tributylphosphorane or reagent combination such as triphenylphosphine (optionally polymer-supported) and DIAD in a suitable solvent such as toluene or THF.

One skilled in the art will recognize that still further permutations of the bond forming steps and functional group manipulations in Schemes 1-4 may be applied with appropriate considerations. Such permutations in the selection of step order are well known in the chemical literature and one skilled in the art may consult the chemical literature for further guidance if desired. One skilled in the art will recognize that in some instances, certain combinations of reagents will be more optimally suited for the particular reactant combinations. One skilled in the art will recognize that other selections of protecting groups and reagents for effecting the various transformations may be made.

### EXAMPLES

### Experimental Procedures

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

All reactions were carried out using continuous stirring under an atmosphere of nitrogen or argon gas unless otherwise noted. When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, commercial solvents were passed through columns packed with 4 Å molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwaves. Reaction progress was monitored using thin-layer chromatography (TLC), liquid chromatography-mass spectrometry (LCMS), high-performance liquid chromatography (H PLC), and/or gas chromatography-mass spectrometry (GCMS) analyses. TLC was performed on pre-coated silica gel plates with a fluorescence indicator (254 nm excitation wavelength) and visualized under UV light and/or with I₂, KMnO₄, CoCl₂, phosphomolybdic acid, and/or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluate was analyzed using a Waters ZQ mass spectrometer scanning in both positive and negative ion modes from 100 to 1200 Da. Other similar instruments were also used. HPLC data were generally acquired on an Agilent 1100 Series instrument, using the columns indicated, acetonitrile/water gradients, and either trifluoroacetic acid or ammonium hydroxide modifiers. GCMS data were acquired using a Hewlett Packard 6890 oven with an HP 6890 injector, HP-1 column (12 m×0.2 mm×0.33 μm), and helium carrier gas. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC), generally using Berger or Thar instruments; columns such as ChiralPAK-AD, -AS, -IC, Chiralcel-OD, or -OJ columns; and CO₂ mixtures with methanol, ethanol, 2-propanol, or acetonitrile, alone or modified using trifluoroacetic acid or propan-2-amine. UV detection was used to trigger fraction collection. For syntheses referencing procedures in other Examples or Methods, purifications may vary: in general, solvents and the solvent ratios used for eluents/gradients were chosen to provide appropriate R_{f}s or retention times.

Mass spectrometry data are reported from LCMS analyses. Mass spectrometry (MS) was performed via atmospheric pressure chemical ionization (APCI), electrospray ionization (ESI), electron impact ionization (EI) or electron scatter ionization (ES) sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, δ) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were generally acquired on a Berger analytical instrument as described above. Optical rotation data were acquired on a PerkinElmer model 343 polarimeter using a 1 dm cell. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially and used without further purification, or were prepared using methods known in the literature.

The terms “concentrated”, “evaporated”, and “concentrated in vacuo” refer to the removal of solvent at reduced pressure on a rotary evaporator with a bath temperature less than 60° C. The abbreviations “min” and “h” stand for “minutes” and “hours,” respectively. The term “TLC” refers to thin-layer chromatography, “room temperature or ambient temperature” means a temperature between 18 to 25° C., “GCMS” refers to gas chromatography-mass spectrometry, “LCMS” refers to liquid chromatography-mass spectrometry, “UPLC” refers to ultra-performance liquid chromatography, “HPLC” refers to high-performance liquid chromatography, and “SFC” refers to supercritical fluid chromatography.

Hydrogenation may be performed in a Parr shaker under pressurized hydrogen gas, or in a Thales-nano H-Cube flow hydrogenation apparatus at full hydrogen and a flow rate between 1-2 mL/min at specified temperature.

HPLC, UPLC, LCMS, GCMS, and SFC retention times were measured using the methods noted in the procedures.

In some examples, chiral separations were carried out to separate enantiomers or diastereomers of certain compounds of the invention (in some examples, the separated enantiomers are designated as ENT-1 and ENT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The compounds and intermediates described below were named using the naming convention provided with ACD/ChemSketch 2019.1.1, File Version C05H41, Build 110712 (Advanced Chemistry Development, Inc., Toronto, Ontario, Canada). The naming convention provided with ACD/ChemSketch 2019.1.1 is well known by those skilled in the art and it is believed that the naming convention provided with ACD/ChemSketch 2019.1.1 generally comports with the IUPAC (International Union for Pure and Applied Chemistry) recommendations on Nomenclature of Organic Chemistry and the CAS Index rules.

### Example 1

### (1R,2S,5S)-N-{(2S)-4-(2,4-Difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

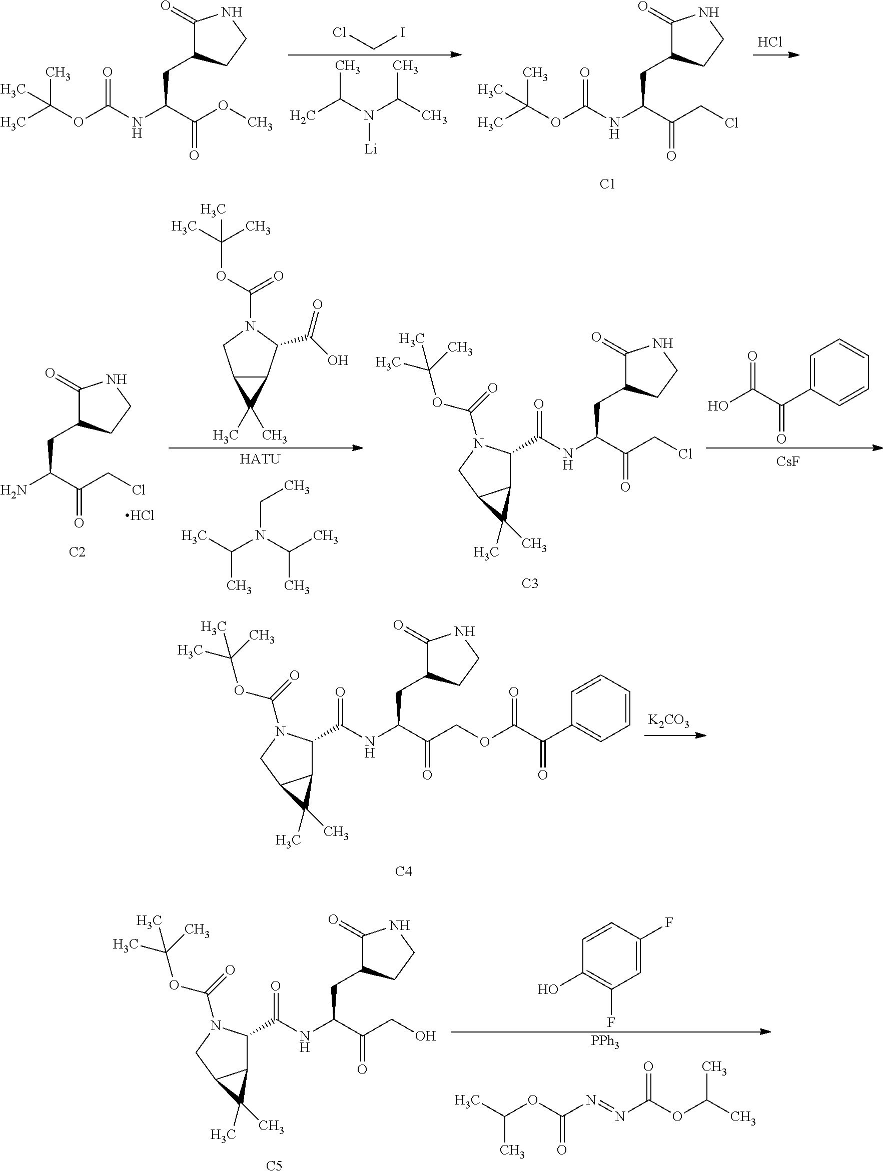

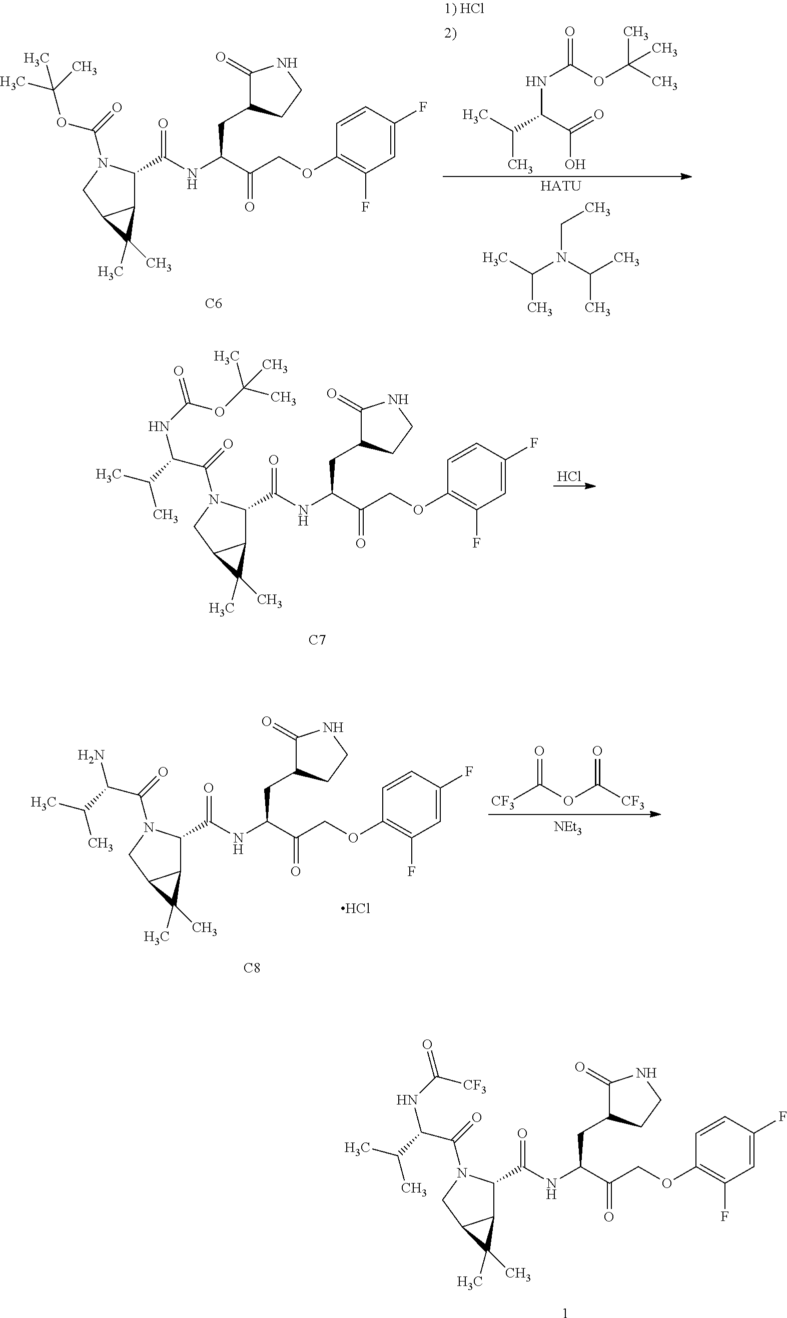

### Step 1. Synthesis of tert-butyl {(2S)-4-chloro-3-oxo-1-[(3S)-2-oxopyrrolidin-3-yl]butan-2-yl}carbamate (C1)

This reaction was carried out in two identical batches. A solution of methyl N-(tert-butoxycarbonyl)-3-[(3S)-2-oxopyrrolidin-3-yl]-L-alaninate (200 g, 698 mmol) and chloro(iodo)methane (739 g, 4.19 mol) in tetrahydrofuran (4.0 L) was cooled to −78° C., whereupon lithium diisopropylamide solution (2.0 M; 2.79 L, 5.58 mol) was added in a drop-wise manner, and stirring was continued at −78° C. for 1 hour. The reaction mixture was quenched at −70° C. via slow, drop-wise addition of a solution of acetic acid (660 mL) in tetrahydrofuran (1.32 L), and was then allowed to warm to 0° C.; at this point, the two batches were combined. After the resulting mixture had been diluted with water (4 L), it was extracted with ethyl acetate (3×4.0 L). The combined organic layers were washed sequentially with aqueous sodium sulfite solution (3 L), aqueous sodium is bicarbonate solution (3 L), and saturated aqueous sodium chloride solution (3 L), then dried over sodium sulfate, filtered, and concentrated in vacuo. Purification of the residue using silica gel chromatography (Gradient: 0% to 80% ethyl acetate in petroleum ether) provided C1 as a white solid. Combined yield: 100 g, 328 mmol, 23%. LCMS m/z 327.0 (chlorine isotope pattern observed) [M+Na⁺]. ¹H NMR (400 MHz, DMSO-d₆) δ 7.66 (br s, 1H), 7.53 (br d, J=7.6 Hz, 1H), 4.61 (AB quartet, J_{AB}=16.8 Hz, Δv_{AB}=9.2 Hz, 2H), 4.16 (ddd, J=11.3, 7.5, 4.1 Hz, 1H), 3.23-3.07 (m, 2H), 2.31-2.19 (m, 1H), 2.19-2.07 (m, 1H), 1.87 (ddd, J=13.9, 10.9, 4.6 Hz, 1H), 1.73-1.55 (m, 2H), 1.39 (s, 9H).

### Step 2. Synthesis of (3S)-3-[(2S)-2-amino-4-chloro-3-oxobutyl]pyrrolidin-2-one, hydrochloride salt (C2)

A solution of hydrogen chloride in 1,4-dioxane (4.0 M; 125 mL, 500 mmol) was added to a 0° C. solution of C1 (10.0 g, 32.8 mmol) in ethyl acetate (100 mL). After the reaction mixture had been stirred for 2 hours at 0° C., it was warmed to room temperature and stirred for an additional hour. It was then concentrated in vacuo, and the residue was azeotroped with methanol, affording C2 as a solid (8.0 g); this material was used directly in the following step. LCMS m/z 205.2 (chlorine isotope pattern observed) [M+H]⁺. ¹H NMR (400 MHz, DMSO-d₆) δ 8.62 (br s, 3H), 7.99 (s, 1H), 4.84 (AB quartet, J_{AB}=17.2 Hz, Δv_{AB}=60.1 Hz, 2H), 4.36-4.25 (m, 1H), 3.28-3.12 (m, 2H; assumed; partially obscured by water peak), 2.65-2.53 (m, 1H), 2.36-2.24 (m, 1H), 2.00 (ddd, component of ABXY system, J=14.7, 7.8, 3.6 Hz, 1H), 1.91 (ddd, component of ABXY system, J=14.8, 9.5, 6.9 Hz, 1H), 1.79-1.66 (m, 1H).

### Step 3. Synthesis of tert-butyl (1R,2S,5S)-2-({(2S)-4-chloro-3-oxo-1-[(3S)-2-oxopyrrolidin-3-yl]butan-2-yl}carbamoyl)-6,6-dimethyl-3-azabicyclo[3.1.0]hexane-3-carboxylate (C3)

O-(7-Azabenzotriazol-1-yl)-N,N,N′N′-tetramethyluronium hexafluorophosphate (HATU; 15.1 g, 39.7 mmol) was added to a solution of C2 (from the previous step; 8.0 g, 532.8 mmol) and (1R,2S,5S)-3-(tert-butoxycarbonyl)-6,6-dimethyl-3-azabicyclo[3.1.0]hexane-2-carboxylic acid (8.47 g, 33.2 mmol) in N,N-dimethylformamide (166 mL). After the reaction mixture had been stirred at 0° C. for 5 minutes, N,N-diisopropylethylamine (12.7 mL, 72.9 mmol) was added in a drop-wise manner, and stirring was continued at 0° C. for 1.5 hours. Water (250 mL) and aqueous citric acid solution (1 M; 100 mL) were then added, followed by ethyl acetate (300 mL); the organic layer was washed with water (2×250 mL), and the combined aqueous layers were extracted with ethyl acetate (3×100 mL). The combined organic layers were dried over sodium sulfate, filtered, concentrated in vacuo, and azeotroped at 50° C. with toluene (2×50 mL). Subsequent purification via silica gel chromatography (Gradient: 0% to 100% ethyl acetate in heptane) provided impure material, which was subjected to a second chromatographic purification on silica gel (Gradient: 0% to 20% methanol in dichloromethane). The resulting material was treated with dichloromethane (approximately 30 mL) and filtered; the filtered solids were washed with dichloromethane (approximately 15 mL), and the combined filtrates were concentrated in vacuo to provide C3 as a gum. From analysis of the ¹H NMR, this material exists as a mixture of rotamers. Yield: 10.4 g, 23.5 mmol, 72% over 2 steps. LCMS m/z 442.4 (chlorine isotope pattern observed) [M+H]+. ¹H NMR (600 MHz, DMSO-d₆) δ [8.69 (d, J=7.5 Hz) and 8.63 (d, J=7.9 Hz), total 1H], [7.69 (br s) and 7.63 (br s), total 1H], [4.63 (s) and 4.60 (AB quartet, J_{AB}=16.8 Hz, Δv_{AB}=42.5 Hz), total 2H], [4.46 (ddd, J=11.3, 7.4, 3.8 Hz) and 4.41 (ddd, J=11.6, 7.8, 3.9 Hz), total 1H], [3.99 (s) and 3.96 (s), total 1H], [3.59 (dd, J=10.8, 5.3 Hz) and 3.52 (dd, J=11.0, 5.1 Hz), total 1H], [3.35-3.28 (m, assumed; obscured by water peak) and 3.26 (d, J=10.8 Hz), total 1H], 3.21-3.13 (m, 1H), 3.13-3.05 (m, 1H), 2.33-2.20 (m, 1H), 2.18-2.10 (m, 1H), 2.01-1.90 (m, 1H), 1.74-1.57 (m, 2H), [1.41 (dd, J=7.6, 5.4 Hz) and 1.39-1.28 (m, assumed), total 2H], [1.37 (s) and 1.32 (s), total 9H], 1.01 (s, 3H), [0.90 (s) and 0.88 (s), total 3H].

### Step 4. Synthesis of tert-butyl (1R,2S,5S)-6,6-dimethyl-2-({(2S)-3-oxo-4-{[oxo(phenyl)acetyl]oxy}-1-[(3S)-2-oxopyrrolidin-3-yl]butan-2-yl}carbamoyl)-3-azabicyclo[3.1.0]hexane-3-carboxylate (C4)

A mixture of oxo(phenyl)acetic acid (4.59 g, 30.6 mmol) and cesium fluoride (8.22 g, 54.1 mmol) in N,N-dimethylformamide (75 mL) was stirred at 65° C. for 5 minutes, whereupon a solution of C3 (10.4 g, 23.5 mmol) in N,N-dimethylformamide (25 mL) was added. After the reaction mixture had been heated at 65° C. for 1.5 hours, it was cooled to room temperature and partitioned between aqueous citric acid solution (1 M; 10 mL) and ethyl acetate (250 mL). The organic layer was washed with water (3×125 mL), and the combined aqueous layers were then extracted with ethyl acetate (2×50 mL). The combined organic layers were dried over sodium sulfate, filtered, concentrated in vacuo, and subjected to chromatography on silica gel (Gradient: 0% to 100% ethyl acetate in heptane). The resulting material was purified via silica gel chromatography (Gradient: 0% to 10% methanol in dichloromethane), affording C4 as a gum. From analysis of the ¹H NMR, this material exists as a mixture of rotamers. Yield: 5.35 g, 9.63 mmol, 41%. LCMS m/z 556.5 [M+H]⁺. ¹H NMR (400 MHz, chloroform-d) 6 [8.81 (br d, J=5.1 Hz) and 8.34 (br d, J=6.3 Hz), total 1H], 8.21-8.13 (m, 2H), 7.70-7.63 (m, 1H), 7.57-7.49 (m, 2H), [6.07 (br s) and 5.74 (br s), total 1H], [5.20 (AB quartet, J_{AB}=17.1 Hz, Δv_{AB}=56.3 Hz) and 5.19 (AB quartet, J_{AB}=16.7 Hz, Δv_{AB}=40.8 Hz), total 2H], [4.68-4.59 (m) and 4.57-4.49 (m), total 1H], [4.14 (s) and 4.10 (s), total 1H], 3.72-3.62 (m, 1H), [3.57 (d, half of AB quartet, J=11.4 Hz) and 3.46-3.33 (m), total 3H], 2.56-2.36 (m, 2H), 2.24-2.11 (m, 1H), 2.08-1.83 (m, 2H), [1.48 (dd, J=7.5, 5.6 Hz), 1.41-1.34 (m), and 1.34-1.27 (m), total 2H], 1.44 (br s, 9H), [1.04 (s) and 1.03 (s), total 3H], [0.93 (s) and 0.91 (s), total 3H].

### Step 5. Synthesis of tert-butyl (1R,2S,5S)-2-({(2S)-4-hydroxy-3-oxo-1-[(3S)-2-oxopyrrolidin-3-yl]butan-2-yl}carbamoyl)-6,6-dimethyl-3-azabicyclo[3.1.0]hexane-3-carboxylate (C5)

A solution of C4 (800 mg, 1.44 mmol) in methanol (9 mL) was treated with potassium carbonate (10 mg, 72 μmol) and stirred at room temperature for 1.5 hours. The reaction mixture was then filtered using a syringe filter, whereupon the eluate was diluted with dichloromethane and concentrated onto silica gel. Silica gel chromatography (Gradient: 0% to 10% methanol in dichloromethane) afforded a clear oil; this material was azeotroped with diethyl ether/heptane to provide C5 as a white solid. From analysis of the ¹H NMR, this material comprised a mixture of rotamers. Yield: 386 mg, 0.911 mmol, 63%. LCMS m/z 424.2 [M+H]⁺. ¹H NMR (400 MHz, chloroform-d) δ [8.55 (br d, J=5.7 Hz) and 8.11 (br d, J=6.6 Hz), total 1H], [5.80 (br s) and 5.56 (br s), total 1H], 4.65-4.51 (m, 1H), 4.50-4.31 (m, 2H), [4.12 (s) and 4.09 (s), total 1H], 3.69-3.61 (m, 1H), [3.57 (d, half of AB quartet, J=11.3 Hz) and 3.45-3.30 (m), total 3H], 2.52-2.36 (m, 2H), 2.20-2.04 (m, 1H), 1.98-1.81 (m, 2H), [1.51-1.45 (m), 1.43-1.33 (m), and 1.33-1.27 (m), total 2H], 1.45 (s, 9H), [1.04 (s) and 1.03 (s), total 3H], [0.93 (s) and 0.91 (s), total 3H].

### Step 6. Synthesis of tert-butyl (1R,2S,5S)-2-({(2S)-4-(2,4-difluorophenoxy)-3-oxo-1-[(3S)-2-oxopyrrolidin-3-yl]butan-2-yl}carbamoyl)-6,6-dimethyl-3-azabicyclo[3.1.0]hexane-3-carboxylate (C6)

To a 0° C. solution of triphenylphosphine (316 mg, 1.20 mmol) in tetrahydrofuran (4.7 mL) was added C5 [300 mg, 0.708 mmol; 100 mg as a solid, and the remaining 200 mg as a solution in tetrahydrofuran (0.3 mL)]. 2,4-Difluorophenol (115 μL, 1.20 mmol) was then added, followed by diisopropyl azodicarboxylate (209 μL, 1.06 mmol). After the reaction mixture had been stirred at 0° C. for 5 minutes, it was allowed to warm to room temperature and stir for 1 hour, then concentrated in vacuo and purified via chromatography on silica gel (Gradient: 0% to 10% methanol in dichloromethane). The resulting material was azeotroped once with heptane, then dissolved in diethyl ether and treated with heptane until the solution became cloudy, whereupon solvents were removed in vacuo to afford C6 as a white solid. From analysis of the ¹H NMR, this material comprised a mixture of rotamers. Yield: 140 mg, 0.261 mmol, 37%. LCMS m/z 536.5 [M+H]⁺. ¹H NMR (600 MHz, chloroform-d) δ [8.42 (br d, J=5.7 Hz) and 8.05 (br d, J=6.8 Hz), total 1H], 7.03-6.93 (m, 1H), 6.89-6.81 (m, 1H), 6.81-6.73 (m, 1H), [5.76 (br s) and 5.55 (br s), total 1H], [4.88 (AB quartet, J_{AB}=17.3 Hz, Δv_{AB}=29.4 Hz) and 4.87 (AB quartet, J_{AB}=16.9 Hz, Δv_{AB}=28.1 Hz), total 2H], [4.81-4.75 (m) and 4.75-4.70 (m), total 1H], [4.14 (s) and 4.08 (s), total 1H], 3.69-3.63 (m, 1H), [3.57 (d, half of AB quartet, J=11.4 Hz) and 3.43 (d, half of AB quartet, J=11.1 Hz), total 1H], 3.41-3.32 (m, 2H), 2.52-2.42 (m, 2H), 2.14-1.87 (m, 3H), [1.49 (d, half of AB quartet, J=7.5 Hz) and 1.46 (d, half of AB quartet, J=7.5 Hz), total 1H], [1.43 (s) and 1.41 (s), total 9H], [1.38-1.34 (m) and 1.34-1.23 (m), total 1H], [1.04 (s) and 1.03 (s), total 3H], [0.93 (s) and 0.91 (s), total 3H].

Step 7. Synthesis of tert-butyl {(2S)-1-[(1R,2S,5S)-2-({(2S)-4-(2,4-difluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3-methyl-1-oxobutan-2-yl}carbamate (C7)

A solution of C6 (220 mg, 0.411 mmol) in dichloromethane (3 mL) was treated with a solution of hydrogen chloride in 1,4-dioxane (4 M; 1.03 mL, 4.11 mmol). After the reaction mixture had been stirred at room temperature for 50 minutes, it was concentrated in vacuo; the residue was triturated three times with diethyl ether to provide deprotected material (1R,2S,5S)-N-{(2S)-4-(2,4-difluorophenoxy)-3-oxo-1-[(3S)-2-oxopyrrolidin-3-yl]butan-2-yl}-6,6-dimethyl-3-azabicyclo[3.1.0]hexane-2-carboxamide, hydrochloride salt as a pale yellow solid, LCMS m/z 436.5 [M+H]⁺.

A 0° C. solution of N-(tert-butoxycarbonyl)-L-valine (98.7 mg, 0.454 mmol) in N,N-dimethylformamide (1.8 mL) was treated with O-(7-azabenzotriazol-1-yl)-N,N,N′N′-tetramethyluronium hexafluorophosphate (HATU; 173 mg, 0.455 mmol), followed by N,N-diisopropylethylamine (0.180 mL, 1.03 mmol). After the reaction mixture had been stirred for 5 minutes at 0° C., (1R,2S,5S)-N-{(2S)-4-(2,4-difluorophenoxy)-3-oxo-1-[(3S)-2-oxopyrrolidin-3-yl]butan-2-yl}-6,6-dimethyl-3-azabicyclo[3.1.0]hexane-2-carboxamide, hydrochloride salt (from above; 50.411 mmol) was added [most of this material was added as a solid; then the flask was rinsed with N,N-dimethylformamide (300 μL) and added to the reaction mixture]. After approximately 30 minutes, the reaction mixture was diluted with water, followed by aqueous citric acid solution (180 μL) and ethyl acetate. The resulting mixture was stirred for 5 minutes, whereupon the aqueous layer was extracted twice with ethyl acetate. The combined organic layers were washed with twice with water and once with saturated aqueous sodium chloride solution, dried over magnesium sulfate, and filtered. After the filtrate had been concentrated onto silica gel, it was purified via silica gel chromatography (Gradient: 0% to 10% methanol in dichloromethane). The resulting material was azeotroped twice with heptane and once with diethyl ether/heptane to afford C7 as a white solid. Yield: 159 mg, 0.251 mmol, 61%. LCMS m/z 635.7 [M+H]⁺. ¹H NMR (600 MHz, chloroform-d) δ 8.25 (br d, J=5.9 Hz, 1H), 6.97 (ddd, J=9.2, 9.2, 5.2 Hz, 1H), 6.85 (ddd, J=11.2, 8.4, 3.0 Hz, 1H), 6.80-6.74 (m, 1H), 5.60 (br s, 1H), 5.06 (br d, J=9.8 Hz, 1H), 4.86 (s, 2H), 4.76-4.70 (m, 1H), 4.34 (s, 1H), 4.14 (dd, J=8.5, 8.5 Hz, 1H), 3.97 (dd, J=10.3, 5.2 Hz, 1H), 3.90 (d, J=10.3 Hz, 1H), 3.41-3.32 (m, 2H), 2.59-2.50 (m, 1H), 2.49-2.41 (m, 1H), 2.06-1.96 (m, 3H), 1.95-1.86 (m, 1H), 1.52-1.48 (m, 1H), 1.47 (d, half of AB quartet, J=7.6 Hz, 1H), 1.40 (s, 9H), 1.05 (s, 3H), 0.97 (d, J=6.7 Hz, 3H), 0.93-0.90 (m, 6H).

### Step 8. Synthesis of (1R,2S,5S)-N-{(2S)-4-(2,4-difluorophenoxy)-3-oxo-1-[(3S)-2-oxopyrrolidin-3-yl]butan-2-yl}-6,6-dimethyl-3-L-valyl-3-azabicyclo[3.1.0]hexane-2-carboxamide, hydrochloride salt (C8)

A solution of hydrogen chloride in 1,4-dioxane (4 M; 0.626 mL, 2.50 mmol) was added to a solution of C7 (159 mg, 0.251 mmol) in dichloromethane (2 mL). After the reaction mixture had been stirred at room temperature for 75 minutes, additional hydrogen chloride in 1,4-dioxane (4 M; 100 μL, 0.40 mmol) was added; stirring was continued for 75 minutes, whereupon the reaction mixture was concentrated in vacuo. The residue was triturated twice with diethyl ether to provide C8 as an off-white/pale-yellow solid, which was progressed directly to the following step. LCMS m/z 535.2 [M+H]⁺. ¹H NMR (400 MHz, DMSO-d₆) δ 8.85 (d, J=8.0 Hz, 1H), 8.14-8.02 (m, 3H), 7.65 (br s, 1H), 7.30 (ddd, J=11.7, 8.8, 3.0 Hz, 1H), 7.04 (ddd, J=9.4, 9.3, 5.3 Hz, 1H), 6.96 (dddd, J=9, 8, 3, 1.4 Hz, 1H), 5.07 (s, 2H), 4.52 (ddd, J=11.6, 7.9, 3.4 Hz, 1H), 4.33 (s, 1H), 3.99-3.90 (m, 1H), 3.80 (dd, component of ABX system, J=10.6, 5.4 Hz, 1H), 3.68 (d, half of AB quartet, J=10.8 Hz, 1H), 3.21-3.12 (m, 1H), 3.12-3.03 (m, 1H), 2.38-2.28 (m, 1H), 2.20-2.04 (m, 2H), 1.99-1.89 (m, 1H), 1.73-1.62 (m, 2H), 1.56 (dd, J=7.7, 5.3 Hz, 1H), 1.38 (d, half of AB quartet, J=7.7 Hz, 1H), 1.04 (s, 3H), 1.01 (d, J=6.9 Hz, 3H), 0.96 (s, 3H), 0.90 (d, J=6.9 Hz, 3H).

### Step 9. Synthesis of (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

To a 0° C. solution of C8 (from the previous step; 50.251 mmol) in dichloromethane (1.5 mL) was added triethylamine (41.6 μL, 0.298 mmol), followed by addition of trifluoroacetic anhydride (42 μL, 0.30 mmol) in a drop-wise manner over 5 minutes. The reaction mixture was stirred at 0° C. for 40 minutes, whereupon it was diluted with dichloromethane and water. The aqueous layer was extracted with dichloromethane, and the combined organic layers were washed with saturated aqueous sodium chloride solution, dried over magnesium sulfate, filtered, and concentrated in vacuo. Chromatography on silica gel (Gradient: 0% to 5% methanol in dichloromethane) was followed by dissolution of the purified material in diethyl ether. Heptane was added to precipitate the product; filtration afforded (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1) as a white solid. Yield: 70 mg, 0.111 mmol, 44% over 2 steps. LCMS m/z 631.5 [M+H]⁺. ¹H NMR (400 MHz, DMSO-d₆) δ 9.82 (d, J=7.7 Hz, 1H), 8.80 (d, J=7.9 Hz, 1H), 7.62 (s, 1H), 7.29 (ddd, J=11.6, 8.8, 3.0 Hz, 1H), 7.03 (ddd, J=9.3, 9.3, 5.3 Hz, 1H), 6.95 (dddd, J=9, 8, 3.1, 1.6 Hz, 1H), 5.07 (AB quartet, J_{AB}=17.9 Hz, Δv_{AB}=17.9 Hz, 2H), 4.47 (ddd, J=11.4, 7.6, 3.6 Hz, 1H), 4.23 (s, 1H), 4.13 (dd, J=10.0, 7.7 Hz, 1H), 3.88 (dd, component of ABX system, J=10.3, 5.3 Hz, 1H), 3.82 (d, half of AB quartet, J=10.3 Hz, 1H), 3.20-3.12 (m, 1H), 3.12-3.02 (m, 1H), 2.46-2.35 (m, 1H), 2.19-2.09 (m, 1H), 2.09-2.01 (m, 1H), 2.01-1.91 (m, 1H), 1.70-1.59 (m, 2H), 1.55 (dd, J=7.6, 5.1 Hz, 1H), 1.35 (d, half of AB quartet, J=7.6 Hz, 1H), 1.02 (s, 3H), 0.94 (d, J=6.7 Hz, 3H), 0.90-0.86 (m, 6H).

### Alternate Synthesis of Example 1

### (1R,2S,5S)-N-{(2S)-4-(2,4-Difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

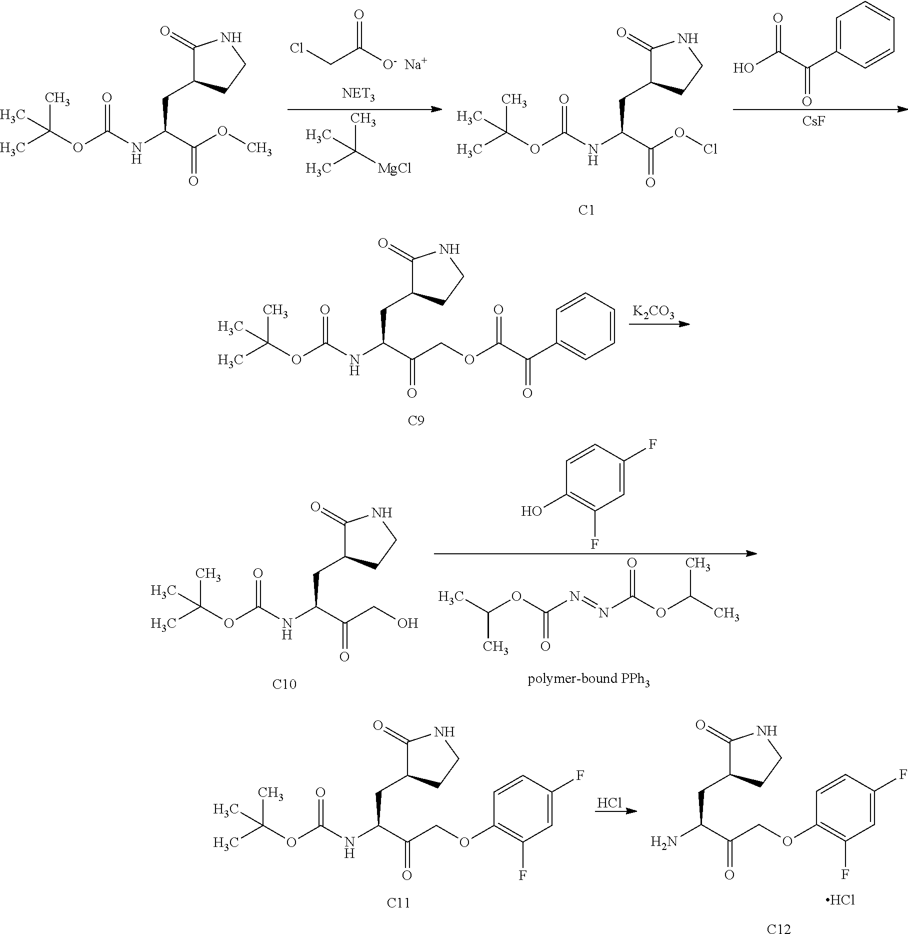

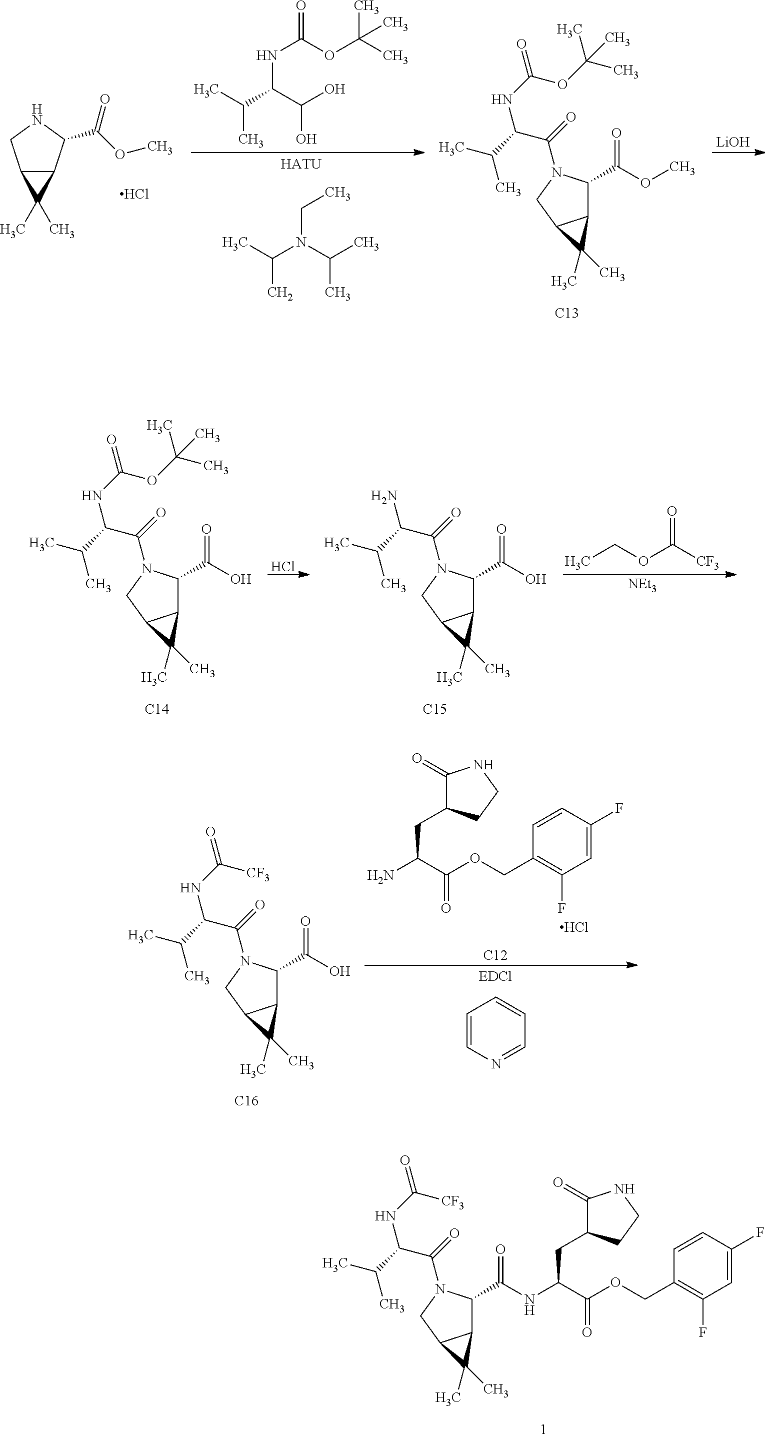

### Step 1. Synthesis of tert-butyl {(2S)-4-chloro-3-oxo-1-[(3S)-2-oxopyrrolidin-3-yl]butan-2-yl}carbamate (C1)

tert-Butylmagnesium chloride (1.7 M; 205 mL, 348 mmol) was slowly added, over approximately 30 minutes, to a 0° C. mixture of sodium chloroacetate (24.4 g, 209 mmol) and triethylamine (29.1 mL, 209 mmol) in tetrahydrofuran (40 mL). The reaction mixture was brought to 15° C. to 25° C., whereupon a solution of methyl N-(tert-butoxycarbonyl)-3-[(3S)-2-oxopyrrolidin-3-yl]-L-alaninate (10.0 g, 34.9 mmol) in tetrahydrofuran (20 mL) was added in a drop-wise manner over 1 hour. After the reaction mixture had been stirred at room temperature (25° C.) for 16 hours, it was cooled to 0° C. and quenched by addition of saturated aqueous ammonium chloride solution. The resulting mixture was extracted with ethyl acetate, and the organic layer was washed with saturated aqueous sodium chloride solution, dried, filtered, and concentrated in vacuo. Purification via chromatography on silica gel (Gradient: 0% to 100% ethyl acetate in petroleum ether) provided C1 as a white solid. Yield: 4.31 g, 14.1 mmol, 40%. LCMS m/z 249.1 (chlorine isotope pattern observed) [(M-2-methylprop-1-ene)+H]⁺. ¹H NMR (400 MHz, chloroform-d) δ 6.39-6.08 (m, 2H), 4.53-4.38 (m, 1H), 4.37 (AB quartet, J_{AB}=16.1 Hz, Δv_{AB}=30.8 Hz, 2H), 3.43-3.29 (m, 2H), 2.51-2.36 (m, 2H), 2.12-2.00 (m, 1H), 1.97-1.80 (m, 2H), 1.44 (br s, 9H).

### Step 2. Synthesis of (3S)-3-[(tert-butoxycarbonyl)amino]-2-oxo-4-[(3S)-2-oxopyrrolidin-3-yl]butyl oxo(phenyl)acetate (C9)

Cesium fluoride (17.2 g, 113 mmol) was added in portions to a solution of oxo(phenyl)acetic acid (8.87 g, 59.1 mmol) in N,N-dimethylformamide (200 mL), and the resulting mixture was stirred at 65° C. for 5 minutes. To this was added a solution of C1 (15.0 g, 49.2 mmol) in N,N-dimethylformamide (40 mL), and heating was continued at 65° C. for 1 hour, whereupon the reaction mixture was cooled to room temperature, diluted with ethyl acetate (500 mL), and washed with water (3×250 mL). The combined aqueous layers were extracted with ethyl acetate (2×250 mL), and then the combined organic layers were dried over sodium sulfate, filtered, and concentrated in vacuo. Silica gel chromatography was carried out twice (Gradient #1: 0% to 10% methanol in dichloromethane; Gradient #2: 0% to 100% ethyl acetate in petroleum ether), affording C9. Yield: 19.9 g, 47.6 mmol, 97%. LCMS m/z 363.3 [(M-2-methylprop-1-ene)+H]⁺. ¹H NMR (400 MHz, chloroform-d) δ 8.19-8.14 (m, 2H), 7.71-7.64 (m, 1H), 7.54 (br dd, J=8.4, 7.2 Hz, 2H), 6.63 (br d, J=6.2 Hz, 1H), 5.70 (br s, 1H), 5.21 (AB quartet, J_{AB}=16.8 Hz, Δv_{AB}=50.5 Hz, 2H), 4.43-4.33 (m, 1H), 3.44-3.31 (m, 2H), 2.57-2.37 (m, 2H), 2.18-2.06 (m, 1H), 2.02-1.83 (m, 2H), 1.46 (s, 9H).

### Step 3. Synthesis of tert-butyl {(2S)-4-hydroxy-3-oxo-1-[(3S)-2-oxopyrrolidin-3-yl]butan-2-yl}carbamate (C10)

A solution of C9 (19.9 g, 47.6 mmol) in methanol (500 mL) was treated with potassium carbonate (329 mg, 2.38 mmol) and stirred at room temperature for 1.5 hours. The reaction mixture was concentrated onto silica gel, and then purified via silica gel chromatography (Gradient: 0% to 100% ethyl acetate in heptane, followed by a gradient of 0% to 20% methanol in dichloromethane); the residue was evaporated from toluene and from diethyl ether to provide C10 as an off-white, sticky foam. Yield: 8.89 g, 31.0 mmol, 65%. LCMS m/z 285.3 [M−H]⁻. ¹H NMR (400 MHz, DMSO-d₆) δ 7.63 (br s, 1H), 7.36 (br d, J=7.9 Hz, 1H), 5.07 (t, J=6.0 Hz, 1H), 4.24 (dd, component of ABX system, J=18.7, 6.0 Hz, 1H), 4.20-4.07 (m, 2H), 3.21-3.07 (m, 2H), 2.30-2.18 (m, 1H), 2.18-2.08 (m, 1H), 1.82 (ddd, J=13.7, 11.2, 4.4 Hz, 1H), 1.72-1.51 (m, 2H), 1.38 (s, 9H).

### Step 4. Synthesis of tert-butyl {(2S)-4-(2,4-difluorophenoxy)-3-oxo-1-[(3S)-2-oxopyrrolidin-3-yl]butan-2-yl}carbamate (C11)

A solution of C10 (1.60 g, 5.59 mmol) in tetrahydrofuran (10 mL) was added to a suspension of polymer-bound triphenylphosphine (approximately 3 mmol/g; 3.17 g, 9.5 mmol) in tetrahydrofuran (30 mL), in a reaction flask equipped with an overhead stirrer. 2,4-Difluorophenol (907 μL, 9.50 mmol) was then added, whereupon the reaction mixture was cooled to 0° C. to 10° C. and diisopropyl azodicarboxylate (1.65 mL, 8.38 mmol) was introduced. After stirring at 0° C. for 10 minutes, the reaction mixture was warmed to room temperature and allowed to stir for 4 hours. It was then diluted with dichloromethane and filtered; the filter cake was rinsed with dichloromethane, and the combined filtrates were concentrated onto silica gel. Purification via silica gel chromatography (Gradient: 0% to 10% methanol in dichloromethane) provided C11. Yield: 1.19 g, 2.99 mmol, 53%. LCMS m/z 421.0 [M+Na⁺]. ¹H NMR (400 MHz, DMSO-d₆) δ 7.66 (br s, 1H), 7.58 (d, J=7.4 Hz, 1H), 7.30 (ddd, J=11.7, 8.8, 3.0 Hz, 1H), 7.05 (ddd, J=9.4, 9.3, 5.4 Hz, 1H), 7.00-6.92 (m, 1H), 5.06 (AB quartet, J_{AB}=18.3 Hz, Δv_{AB}=5.4 Hz, 2H), 4.19 (ddd, J=11.2, 7.4, 4.0 Hz, 1H), 3.22-3.08 (m, 2H), 2.33-2.22 (m, 1H), 2.22-2.11 (m, 1H), 1.89 (ddd, J=13.8, 11.0, 4.6 Hz, 1H), 1.74-1.57 (m, 2H), 1.40 (s, 9H).

### Step 5. Synthesis of (3S)-3-[(2S)-2-amino-4-(2,4-difluorophenoxy)-3-oxobutyl]pyrrolidin-2-one, hydrochloride salt (C12)

A solution of C11 (1.19 g, 2.99 mmol) in dichloromethane (17 mL) was treated with a solution of hydrogen chloride in 1,4-dioxane (4 M; 7.48 mL, 29.9 mmol) and stirred at room temperature for 1 hour. The reaction mixture was concentrated in vacuo, and the residue was azeotroped with heptane, then triturated twice with diethyl ether to afford C12 as a light-pink solid. Yield: 1.08 g, assumed quantitative. LCMS m/z 299.3 [M+H]⁺. ¹H NMR (400 MHz, DMSO-d₆) δ 8.54 (br s, 3H), 8.01 (s, 1H), 7.34 (ddd, J=11.7, 8.8, 3.0 Hz, 1H), 7.20 (ddd, J=9.4, 9.4, 5.4 Hz, 1H), 7.06-6.99 (m, 1H), 5.20 (AB quartet, J_{AB}=18.0 Hz, Δv_{AB}=44.8 Hz, 2H), 4.41-4.30 (m, 1H), 3.26-3.17 (m, 2H), 2.67-2.55 (m, 1H), 2.38-2.26 (m, 1H), 2.06 (ddd, component of ABXY system, J=14.8, 7.9, 3.0 Hz, 1H), 1.91 (ddd, component of ABXY system, J=14.8, 9.9, 6.7 Hz, 1H), 1.82-1.70 (m, 1H).

### Step 6. Synthesis of methyl (1R,2S,5S)-3-[N-(tert-butoxycarbonyl)-L-valyl]-6,6-dimethyl-3-azabicyclo[3.1.0]hexane-2-carboxylate (C13)

A 0° C. solution of N-(tert-butoxycarbonyl)-L-valine (69.7 g, 321 mmol) in a mixture of acetonitrile and N,N-dimethylformamide (10:1, 1.10 L) was treated with 0-(7-azabenzotriazol-1-yl)-N,N,N′N′-tetramethyluronium hexafluorophosphate (HATU; 122 g, 321 mmol), followed by N,N-diisopropylethylamine (127 mL, 729 mmol). After the reaction mixture had been stirred for 5 minutes, methyl (1R,2S,5S)-6,6-dimethyl-3-azabicyclo[3.1.0]hexane-2-carboxylate, hydrochloride salt (60.0 g, 292 mmol) was added, and stirring was continued at 0° C. for 1 hour. The reaction mixture was then diluted with aqueous citric acid solution (1 N; 50 mL) and water (100 mL), stirred for 2 minutes, and concentrated in vacuo to approximately one-half of the initial volume. The resulting mixture was partitioned between ethyl acetate and water, and the aqueous layer was extracted three times with ethyl acetate. The combined organic layers were then washed three times with water and once with saturated aqueous sodium chloride solution, dried over sodium sulfate, filtered, and concentrated in vacuo. The residue was stirred in a minimal amount of ethyl acetate, and then filtered; the insoluble material was washed with ethyl acetate until it was white. The combined filtrates were concentrated under reduced pressure and then subjected to silica gel chromatography (Eluent: 1:1 ethyl acetate/heptane), affording C13 as a yellow oil. Yield: 109 g, quantitative. LCMS m/z 369.3 [M+H]⁺. ¹H NMR (400 MHz, chloroform-d) δ 5.08 (d, J=9.6 Hz, 1H), 4.45 (s, 1H), 4.11 (dd, J=9.7, 7.8 Hz, 1H), 3.95 (d, half of AB quartet, J=10.1 Hz, 1H), 3.86 (dd, component of ABX system, J=10.2, 4.8 Hz, 1H), 3.74 (s, 3H), 2.04-1.93 (m, 1H), 1.50-1.41 (m, 2H), 1.40 (s, 9H), 1.04 (s, 3H), 1.00 (d, J=6.8 Hz, 3H), 0.95 (d, J=6.8 Hz, 3H), 0.93 (s, 3H).

### Step 7. Synthesis of (1R,2S,5S)-3-[N-(tert-butoxycarbonyl)-L-valyl]-6,6-dimethyl-3-azabicyclo[3.1.0]hexane-2-carboxylic acid (C14)

An aqueous solution of lithium hydroxide (2.0 M; 436 mL, 872 mmol) was added to a solution of C13 (107 g, 290 mmol) in tetrahydrofuran (730 mL). After the resulting mixture had been stirred at room temperature for approximately 2 hours, it was diluted with water and ethyl acetate, then treated with 1 M aqueous sodium hydroxide solution. The aqueous layer was washed with ethyl acetate, and the combined organic layers were extracted three times with 1 M aqueous sodium hydroxide solution, until LCMS analysis indicated that C14 had been completely removed from the organic layer. Acidification of the combined aqueous layers to pH 2 was carried out by addition of concentrated hydrochloric acid, whereupon the mixture was extracted three times with ethyl acetate. The combined organic layers were washed with saturated aqueous sodium chloride solution, dried over sodium sulfate, filtered, and concentrated; trituration of the residue with heptane afforded C14 as a white solid. Yield: 92.8 g, 262 mmol, 90%. LCMS m/z 355.3 [M+H]⁺. ¹H NMR (400 MHz, methanol-d₄) δ 4.32 (s, 1H), 4.05 (d, half of AB quartet, J=10.5 Hz, 1H), 4.01 (d, J=9.0 Hz, 1H), 3.88 (dd, component of ABX system, J=10.4, 5.3 Hz, 1H), 2.03-1.91 (m, 1H), 1.57 (dd, component of ABX system, J=7.5, 5.2 Hz, 1H), 1.50 (d, half of AB quartet, J=7.5 Hz, 1H), 1.41 (s, 9H), 1.08 (s, 3H), 0.99 (d, J=6.8 Hz, 3H), 0.97-0.94 (m, 6H).

### Step 8. Synthesis of (1R,2S,5S)-6,6-dimethyl-3-L-valyl-3-azabicyclo[3.1.0]hexane-2-carboxylic acid, hydrochloride salt (C15)

To a solution of C14 (82.8 g, 234 mmol) in dichloromethane (230 mL) was added a solution of hydrogen chloride in 1,4-dioxane (4.0 M; 409 mL, 1.64 mol). The reaction mixture was stirred overnight at room temperature, whereupon it was concentrated in vacuo, providing C15 as a white foam. This material was used directly in the following step. LCMS m/z 255.3 [M+H]⁺. ¹H NMR (400 MHz, methanol-d₄) δ 4.42 (s, 1H), 4.05 (d, J=4.8 Hz, 1H), 3.89 (dd, component of ABX system, J=10.5, 5.2 Hz, 1H), 3.74 (d, half of AB quartet, J=10.5 Hz, 1H), 2.36-2.25 (m, 1H), 1.62 (dd, component of ABX system, J=7.5, 5.1 Hz, 1H), 1.57 (d, half of AB quartet, J=7.6 Hz, 1H), 1.16 (d, J=7.0 Hz, 3H), 1.10 (s, 3H), 1.04 (d, J=6.9 Hz, 3H), 1.01 (s, 3H).

### Step 9. Synthesis of (1R,2S,5S)-6,6-dimethyl-3-[N-(trifluoroacetyl)-L-valyl]-3-azabicyclo[3.1.0]hexane-2-carboxylic acid (C16)

A solution of C15 (from the previous step; 5234 mmol) in methanol (230 mL) was cooled to 0° C., treated with triethylamine (66.7 mL, 479 mmol), and stirred for 5 minutes, whereupon ethyl trifluoroacetate (36.1 mL, 303 mmol) was slowly added. After the reaction mixture had been allowed to stir at room temperature for 90 minutes, it was concentrated in vacuo. The residue was diluted with water, 1 M aqueous sodium hydroxide solution, and ethyl acetate, and the resulting organic layer was extracted twice with 1 M aqueous sodium hydroxide solution. The combined aqueous layers were acidified to pH 2 by addition of 1 M hydrochloric acid, then extracted three times with ethyl acetate. These three organic layers were combined, washed with water and with saturated aqueous sodium chloride solution, dried over sodium sulfate, filtered, and concentrated in vacuo, affording C16 as a white foam. Yield: 73.4 g, 210 mmol, 90% over 2 steps. LCMS m/z 351.3 [M+H]⁺. ¹H NMR (400 MHz, DMSO-d₆) δ 12.65 (v br s, 1H), 9.82 (d, J=7.7 Hz, 1H), 4.16 (dd, J=9.9, 7.9 Hz, 1H), 4.12 (s, 1H), 3.86 (d, half of AB quartet, J=10.4 Hz, 1H), 3.81 (dd, component of ABX system, J=10.5, 5.0 Hz, 1H), 2.18-2.05 (m, 1H), 1.54 (dd, component of ABX system, J=7.7, 4.6 Hz, 1H), 1.42 (d, half of AB quartet, J=7.5 Hz, 1H), 1.02 (s, 3H), 0.95 (d, J=6.7 Hz, 3H), 0.89 (d, J=6.6 Hz, 3H), 0.84 (s, 3H).

Step 10. Synthesis of (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

A suspension of C16 (358 mg, 1.02 mmol) and C12 (342 mg, 1.02 mmol) in acetonitrile (5 mL) was cooled to 0° C., and treated with 1-[3-(dimethylamino)propyl]-3-ethylcarbodiimide hydrochloride (EDCl; 196 mg, 1.02 mmol), followed by drop-wise addition of pyridine (0.231 mL, 2.86 mmol). After the reaction mixture had been stirred at room temperature for 1 hour and 50 minutes, it was partitioned between ethyl acetate and water; the resulting mixture was washed with 1 M hydrochloric acid, and the aqueous layer was extracted once with ethyl acetate. The combined organic layers were washed sequentially with saturated aqueous sodium bicarbonate solution and saturated aqueous sodium chloride solution, dried over magnesium sulfate, filtered, and concentrated in vacuo. The resulting material was combined with purified 1 (212 mg, 0.336 mmol) from a similar reaction and subjected to silica gel chromatography (Gradient: 0% to 10% methanol in dichloromethane). The isolated material was dissolved in diethyl ether and treated with heptane to precipitate the product; filtration afforded (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1) as a white solid. Yield: 490 mg, 0.777 mmol, 43%, yield adjusted for added material. LCMS m/z 631.2 [M+H]⁺. ¹H NMR (400 MHz, DMSO-d₆) δ9.82 (d, J=7.7 Hz, 1H), 8.80 (d, J=7.9 Hz, 1H), 7.62 (s, 1H), 7.29 (ddd, J=11.7, 8.8, 3.0 Hz, 1H), 7.03 (ddd, J=9.4, 9.3, 5.4 Hz, 1H), 6.99-6.91 (m, 1H), 5.07 (AB quartet, J_{AB}=17.9 Hz, Δv_{AB}=18.0 Hz, 2H), 4.46 (ddd, J=11.6, 7.7, 3.6 Hz, 1H), 4.23 (s, 1H), 4.13 (dd, J=10.0, 7.7 Hz, 1H), 3.88 (dd, component of ABX system, J=10.3, 5.3 Hz, 1H), 3.82 (d, half of AB quartet, J=10.3 Hz, 1H), 3.20-3.12 (m, 1H), 3.12-3.02 (m, 1H), 2.46-2.35 (m, 1H), 2.19-2.10 (m, 1H), 2.10-2.00 (m, 1H), 2.00-1.91 (m, 1H), 1.70-1.59 (m, 2H), 1.54 (dd, J=7.5, 5.3 Hz, 1H), 1.35 (d, half of AB quartet, J=7.6 Hz, 1H), 1.02 (s, 3H), 0.94 (d, J=6.7 Hz, 3H), 0.90-0.86 (m, 6H).

This material was combined with an additional purified sample of 1 (228 mg, 0.361 mmol), slurried with ethanol (15 mL), and treated with water (7.5 mL). The resulting mixture was stirred for 10 minutes, whereupon heptane (7.5 mL) was added in a drop-wise manner with stirring. The vessel was capped, and stirring was continued at room temperature for 3 days. Filtration, followed by rinsing of the filter cake with a mixture of ethanol and water (2:1, 20 mL), followed by heptane (5 mL), afforded (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1) as a crystalline solid (440 mg, 0.698 mmol). LCMS m/z 631.2 [M+H]⁺. ¹H NMR (400 MHz, DMSO-d₆) δ 9.82 (d, J=7.7 Hz, 1H), 8.80 (d, J=7.9 Hz, 1H), 7.62 (s, 1H), 7.29 (ddd, J=11.7, 8.8, 3.0 Hz, 1H), 7.04 (ddd, J=9.4, 9.3, 5.4 Hz, 1H), 6.95 (dddd, J=9, 8, 3, 1.5 Hz, 1H), 5.07 (AB quartet, J_{AB}=17.9 Hz, Δv_{AB}=17.9 Hz, 2H), 4.47 (ddd, J=11.6, 7.7, 3.6 Hz, 1H), 4.23 (s, 1H), 4.13 (dd, J=10.0, 7.7 Hz, 1H), 3.88 (dd, component of ABX system, J=10.3, 5.2 Hz, 1H), 3.82 (d, half of AB quartet, J=10.3 Hz, 1H), 3.21-3.12 (m, 1H), 3.12-3.02 (m, 1H), 2.46-2.35 (m, 1H), 2.20-2.09 (m, 1H), 2.09-2.02 (m, 1H), 2.00-1.91 (m, 1H), 1.71-1.59 (m, 2H), 1.55 (dd, J=7.6, 5.1 Hz, 1H), 1.35 (d, half of AB quartet, J=7.6 Hz, 1H), 1.02 (s, 3H), 0.94 (d, J=6.7 Hz, 3H), 0.90-0.86 (m, 6H).

### Examples 2 and 1

(1R,2S,5S)-N-{(2R)-4-(2,4-Difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (2) and (1R,2S,5S)-N-{(2S)-4-(2,4-Difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

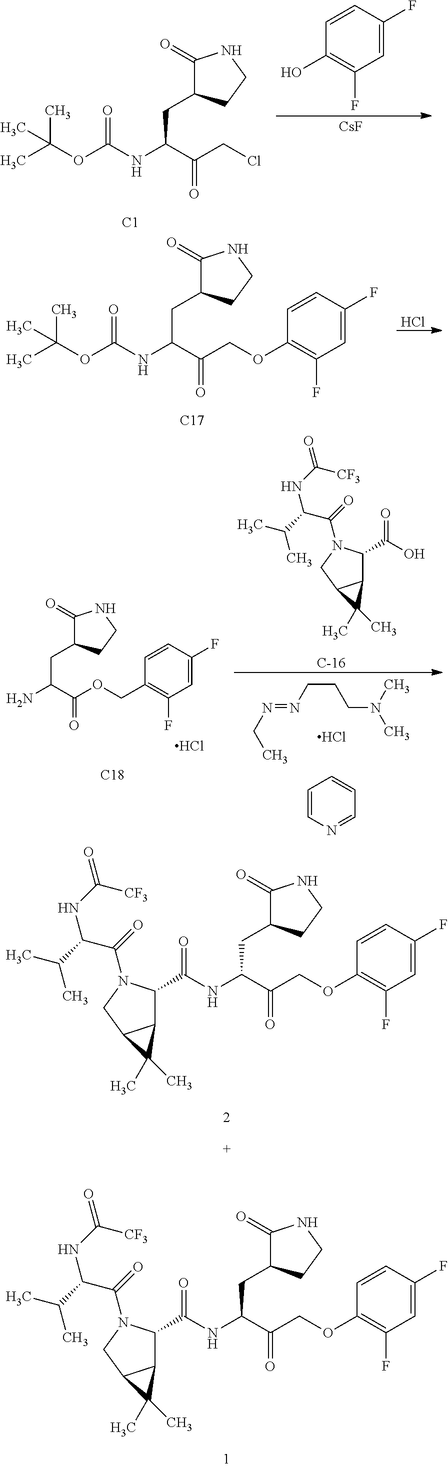

### Step 1. Synthesis of tert-butyl {4-(2,4-difluorophenoxy)-3-oxo-1-[(3S)-2-oxopyrrolidin-3-yl]butan-2-yl}carbamate (C17)

A solution of 2,4-difluorophenol (512 mg, 3.94 mmol) in N,N-dimethylformamide (7 mL) was treated portion-wise with cesium fluoride (1.15 g, 7.57 mmol). After the resulting mixture had been stirred at 65° C. for 5 minutes, a solution of C1 (1.00 g, 3.28 mmol) in N,N-dimethylformamide (3 mL) was added. The reaction mixture was heated at 65° C. for 1 hour, whereupon additional N,N-dimethylformamide (1 mL) was introduced and stirring was continued at 65° C. for 45 minutes. The reaction mixture was then cooled to room temperature, treated with a 1:1 mixture of saturated aqueous potassium bicarbonate solution and ice, and diluted with ethyl acetate and additional aqueous potassium bicarbonate solution. The aqueous layer was extracted with ethyl acetate, and the combined organic layers were washed with saturated aqueous sodium chloride solution, dried over magnesium sulfate, filtered, and concentrated in vacuo onto silica gel. Chromatography on silica gel (Gradient: 0% to 10% methanol in dichloromethane), followed by azeotroping twice with heptane and once with a mixture of diethyl ether and heptane, provided C17 as a very dark orange solid. By ¹H NMR analysis, this material consisted of a mixture of diastereomers. Yield: 580 mg, 1.46 mmol, 44%. LCMS m/z 399.0 [M+H]⁺. ¹H NMR (400 MHz, DMSO-d₆) δ 7.66 (br s, 1H), [7.58 (d, J=7.4 Hz) and 7.54 (d, J=7.3 Hz), total 1H], 7.34-7.25 (m, 1H), 7.10-6.99 (m, 1H), 6.99-6.90 (m, 1H), 5.12-4.99 (m, 2H), [4.40-4.30 (m) and 4.19 (ddd, J=10.8, 7.3, 4.0 Hz), total 1H], 3.23-3.07 (m, 2H), [2.35-2.06 (m) and 1.89 (ddd, J=13.8, 11.1, 4.6 Hz), total 3H], [1.74-1.58 (m) and 1.58-1.47 (m), total 2H], 1.40 (s, 9H).

### Step 2. Synthesis of (3S)-3-[2-amino-4-(2,4-difluorophenoxy)-3-oxobutyl]pyrrolidin-2-one, hydrochloride salt (C18)

A solution of C17 (580 mg, 1.46 mmol) in dichloromethane (8 mL) was treated with a solution of hydrogen chloride in 1,4-dioxane (4 M; 3.64 mL, 14.6 mmol) and then stirred at room temperature. After 15 minutes, methanol (1 mL) was added to enhance solubility; after a further 45 minutes, the reaction mixture was concentrated in vacuo and azeotroped with heptane. The resulting gum was triturated twice with diethyl ether to provide C18 as a dark orange/brown solid (528 mg). This material was used in the following step. LCMS m/z 299.0 [M+H]⁺.

Step 3. Synthesis of (1R,2S,5S)-N-{(2R)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (2) and (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1)

A suspension of C16 (552 mg, 1.58 mmol) and C18 (from the previous step; ≤1.46 mmol) in acetonitrile (8 mL) was cooled to 0° C. and treated with 1-[3-(dimethylamino)propyl]-3-ethylcarbodiimide hydrochloride (302 mg, 1.58 mmol), followed by drop-wise addition of pyridine (0.357 mL, 4.41 mmol). The reaction mixture was stirred at room temperature for 2 hours, whereupon it was diluted with ethyl acetate and water, and washed with 1 M hydrochloric acid. The aqueous layer was extracted with ethyl acetate, and the combined organic layers were washed sequentially with saturated aqueous sodium bicarbonate solution and saturated aqueous sodium chloride solution, dried over magnesium sulfate, filtered, and concentrated in vacuo onto silica gel. Silica gel chromatography (Gradient: 0% to 10% methanol in dichloromethane) afforded a colorless glass that was azeotroped with heptane, then dissolved in minimal tert-butyl methyl ether. Addition of heptane provided a precipitate, whereupon the mixture was concentrated in vacuo. The component diastereomers of this material were separated using supercritical fluid chromatography (Column: Chiral Technologies Chiralpak IC, 30×250 mm, 5 μm; Mobile phase: 4:1 carbon dioxide/2-propanol; Flow rate: 80 mL/minute; Back pressure: 100 bar). The first-eluting diastereomer was (1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (1), obtained as a white solid. Yield: 228 mg, 0.362 mmol, 25% over 2 steps. Retention time: 4.48 minutes (Analytical conditions. Column: Chiral Technologies Chiralpak IC, 4.6×250 mm, 5 μm; Mobile phase A: carbon dioxide; Mobile phase B: methanol; Gradient: 5% B for 1.00 minute, then 5% to 60% B over 8.00 minutes; Flow rate: 3.0 mL/minute; Back pressure: 120 bar).

The second-eluting diastereomer was azeotroped with heptane, then with a mixture of diethyl ether and heptane, to provide (1R,2S,5S)-N-{(2R)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide (2) as a white solid. Yield: 91.2 mg, 0.145 mmol, 10% over 2 steps. LCMS m/z 631.6 [M+H]⁺. ¹H NMR (400 MHz, DMSO-d₆) δ 9.78 (d, J=7.6 Hz, 1H), 8.90 (d, J=7.2 Hz, 1H), 7.66 (s, 1H), 7.33-7.23 (m, 1H), 7.01-6.88 (m, 2H), 5.08 (AB quartet, J_{AB}=18.4 Hz, Δv_{AB}=20.3 Hz, 2H), 4.55-4.46 (m, 1H), 4.24 (s, 1H), 4.14 (dd, J=9.8, 7.8 Hz, 1H), 3.90 (dd, component of ABX system, J=10.4, 5.3 Hz, 1H), 3.83 (d, half of AB quartet, J=10.4 Hz, 1H), 3.20-3.08 (m, 2H), 2.31-2.09 (m, 3H), 2.09-1.98 (m, 1H), 1.75-1.59 (m, 2H), 1.57 (dd, J=7.6, 5.2 Hz, 1H), 1.38 (d, J=7.5 Hz, 1H), 1.03 (s, 3H), 0.94 (d, J=6.7 Hz, 3H), 0.89 (s, 3H), 0.87 (d, J=6.6 Hz, 3H). Retention time: 5.86 minutes (Analytical conditions identical to those used for 1).

### Example 3

### (1R,2S,5S)-N-{(2S)-4-(2,4-Difluorophenoxy)-3-oxo-1-[(3S)-2-oxopyrrolidin-3-yl]butan-2-yl}-6,6-dimethyl-3-{3-methyl-N-[(trifluoromethyl)sulfonyl]-L-valyl}-3-azabicyclo[3.1.0]hexane-2-carboxamide (3)

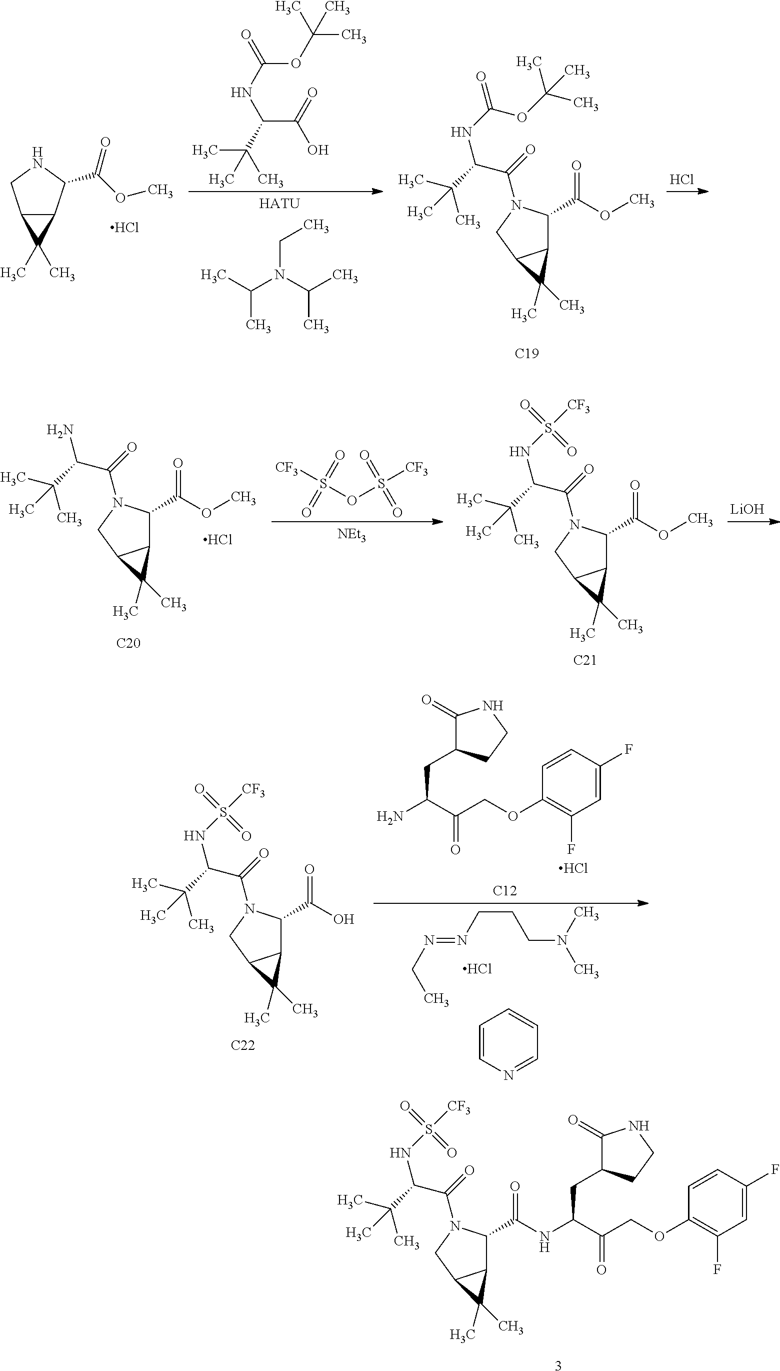

### Step 1. Synthesis of methyl (1R,2S,5S)-3-[N-(tert-butoxycarbonyl)-3-methyl-L-valyl]-6,6-dimethyl-3-azabicyclo[3.1.0]hexane-2-carboxylate (C19)

To a room temperature solution of methyl (1R,2S,5S)-6,6-dimethyl-3-azabicyclo[3.1.0]hexane-2-carboxylate, hydrochloride salt (50.0 g, 243 mmol) and N-(tert-butoxycarbonyl)-3-methyl-L-valine (61.8 g, 267 mmol) in a mixture of acetonitrile and N,N-dimethylformamide (9:1, 970 mL) was added O-(7-azabenzotriazol-1-yl)-N,N,N′,N′-tetramethyluronium hexafluorophosphate (HATU; 102 g, 268 mmol) and N,N-diisopropylethylamine (127 mL, 729 mmol). The reaction mixture was stirred at room temperature overnight, whereupon it was concentrated to 50% of its original volume and partitioned between water (200 mL) and ethyl acetate (200 mL). The aqueous layer was extracted with ethyl acetate (2×100 mL), and the combined organic layers were washed sequentially with water (200 mL), hydrochloric acid (1 M; 100 mL), and saturated aqueous sodium chloride solution (100 mL), dried over sodium sulfate, filtered, and concentrated in vacuo. Chromatography on silica gel (Gradient: 30% to 50% ethyl acetate in heptane) provided C19 as an oil. Yield: 89.5 g, 234 mmol, 96%. LCMS m/z 383.3 [M+H]⁺. ¹H NMR (400 MHz, DMSO-d₆) δ 6.73 (br d, J=9.3 Hz, 1H), 4.21 (s, 1H), 4.05 (d, J=9.4 Hz, 1H), 3.93 (d, half of AB quartet, J=10.4 Hz, 1H), 3.79 (dd, component of ABX system, J=10.3, 5.3 Hz, 1H), 3.65 (s, 3H), 1.52 (dd, component of ABX system, J=7.6, 5.2 Hz, 1H), 1.41 (d, half of AB quartet, J=7.6 Hz, 1H), 1.35 (s, 9H), 1.01 (s, 3H), 0.93 (s, 9H), 0.85 (s, 3H).

### Step 2. Synthesis of methyl (1R,2S,5S)-6,6-dimethyl-3-(3-methyl-L-valyl)-3-azabicyclo[3.1.0]hexane-2-carboxylate, hydrochloride salt (C20)

A solution of hydrogen chloride in 1,4-dioxane (4 M; 85 mL, 340 mmol) was added to a solution of C19 (26.0 g, 68.0 mmol) in dichloromethane (136 mL), and the reaction mixture was stirred at room temperature for 18 hours. It was then concentrated in vacuo; trituration of the residue with diethyl ether afforded C20 as a white solid. Yield: 21.0 g, 65.9 mmol, 97%. ¹H NMR (400 MHz, methanol-d₄) δ 4.46 (s, 1H), 3.99-3.92 (m, 2H), 3.79-3.73 (m, 1H), 3.75 (s, 3H), 1.62 (dd, component of ABX system, J=7.6, 5.4 Hz, 1H), 1.55 (d, half of AB quartet, J=7.6 Hz, 1H), 1.15 (s, 9H), 1.09 (s, 3H), 1.02 (s, 3H).

### Step 3. Synthesis of methyl (1R,2S,5S)-6,6-dimethyl-3-{3-methyl-N-[(trifluoromethyl)sulfonyl]-L-valyl}-3-azabicyclo[3.1.0]hexane-2-carboxylate (C21)

Triethylamine (8.33 mL, 59.8 mmol) was added to a −78° C. solution of C20 (6.35 g, 19.9 mmol) in dichloromethane (100 mL), in a flame-dried Schlenk flask. After the resulting mixture had been stirred for 5 minutes, trifluoromethanesulfonic anhydride (3.52 mL, 20.9 mmol) was added drop-wise, and the reaction mixture was stirred for 1 hour at −78° C., at which point LCMS analysis indicated conversion to C21: LCMS m/z 415.3 [M+H]⁺. The reaction mixture was warmed to room temperature and diluted with ice, whereupon the aqueous layer was extracted twice with dichloromethane. The combined organic layers were washed with saturated aqueous sodium chloride solution, dried over sodium sulfate, filtered, and concentrated in vacuo. Chromatography on silica gel (Gradient: 0% to 60% ethyl acetate in heptane) provided C21 as an oil. Yield: 7.0 g, 17 mmol, 85%. ¹H NMR (400 MHz, DMSO-d₆), from a smaller-scale reaction carried out using the same procedure: δ 9.65 (d, J=9.0 Hz, 1H), 4.24 (s, 1H), 3.91 (d, J=9.0 Hz, 1H), 3.87 (dd, J=10.5, 5.6 Hz, 1H), 3.67 (s, 3H), 3.55 (d, J=10.5 Hz, 1H), 1.57 (dd, component of ABX system, J=7.7, 5.5 Hz, 1H), 1.48 (d, half of AB quartet, J=7.7 Hz, 1H), 1.02 (s, 12H), 0.88 (s, 3H).

### Step 4. Synthesis of (1R,2S,5S)-6,6-dimethyl-3-{3-methyl-N-[(trifluoromethyl)sulfonyl]-L-valyl}-3-azabicyclo[3.1.0]hexane-2-carboxylic acid (C22)

To a solution of C21 (7.0 g, 17 mmol) in tetrahydrofuran (42 mL) was added an aqueous solution of lithium hydroxide (2 M; 25.3 mL, 50.6 mmol), and the reaction mixture was stirred at room temperature for 2 hours, whereupon LCMS analysis indicated the presence of C22: LCMS m/z 401.3 [M+H]⁺. The reaction mixture was diluted with water and ethyl acetate, then treated with 1 M aqueous sodium hydroxide solution; the separated aqueous layer was subsequently acidified to a pH of approximately 2 by addition of 1 M hydrochloric acid and extracted three times with ethyl acetate. These three extracts were combined, washed with saturated aqueous sodium chloride solution, dried over sodium sulfate, filtered, and concentrated in vacuo, affording C22 as a solid. Yield: 6.10 g, 15.2 mmol, 89%. ¹H NMR (400 MHz, DMSO-d₆) δ 9.63 (d, J=9.0 Hz, 1H), 4.16 (s, 1H), 3.90 (d, J=9.0 Hz, 1H), 3.84 (dd, J=10.4, 5.6 Hz, 1H), 3.53 (d, J=10.4 Hz, 1H), 1.55 (dd, component of ABX system, J=7.7, 5.4 Hz, 1H), 1.45 (d, half of AB quartet, J=7.7 Hz, 1H), 1.02 (s, 12H), 0.88 (s, 3H).

Step 5. Synthesis of (1R,2S,5S)-N-{(2S)-4-(2,4-difluorophenoxy)-3-oxo-1-[(3S)-2-oxopyrrolidin-3-yl]butan-2-yl}-6,6-dimethyl-3-{3-methyl-N-[(trifluoromethyl)sulfonyl]-L-valyl}-3-azabicyclo[3.1.0]hexane-2-carboxamide (3)

To a 0° C. solution of C22 (125 mg, 0.312 mmol) and C12 (125 mg, 0.373 mmol) in acetonitrile (3 mL) was added 1-[3-(dimethylamino)propyl]-3-ethylcarbodiimide hydrochloride (65.8 mg, 0.343 mmol), followed by drop-wise addition of pyridine (70.7 μL, 0.874 mmol). The reaction mixture was stirred 0° C. for 20 minutes, then at room temperature for 3 hours, whereupon it was diluted with ethyl acetate and water. The resulting mixture was washed with 1 M hydrochloric acid, and the aqueous layer was extracted with ethyl acetate. The combined organic layers were washed with saturated aqueous sodium chloride solution, dried over magnesium sulfate, filtered, and concentrated in vacuo. Silica gel chromatography (Gradient: 0% to 10% methanol in dichloromethane) provided an oil; this was dissolved in diethyl ether. Addition of heptane resulted in precipitation of a white solid. Removal of solvents in vacuo afforded (1R,2S,5S)-N-{(2S)-4-(2,4-difluorophenoxy)-3-oxo-1-[(3S)-2-oxopyrrolidin-3-yl]butan-2-yl}-6,6-dimethyl-3-{3-methyl-N-[(trifluoromethyl)sulfonyl]-L-valyl}-3-azabicyclo[3.1.0]hexane-2-carboxamide (3) as a white solid. Yield: 128 mg, 0.188 mmol, 60%. LCMS m/z 681.2 [M+H]⁺. ¹H NMR (600 MHz, DMSO-d₆) δ 9.64 (d, J=9.1 Hz, 1H), 8.77 (d, J=8.3 Hz, 1H), 7.60 (s, 1H), 7.33-7.25 (m, 1H), 7.08-7.00 (m, 1H), 6.99-6.92 (m, 1H), 5.08 (AB quartet, J_{AB}=18.0 Hz, Δv_{AB}=11.5 Hz, 2H), 4.58-4.50 (m, 1H), 4.28 (s, 1H), 3.94-3.84 (m, 2H), 3.50 (d, J=10.4 Hz, 1H), 3.19-3.11 (m, 1H), 3.09-3.02 (m, 1H), 2.44-2.35 (m, 1H), 2.19-2.10 (m, 1H), 2.01-1.91 (m, 1H), 1.71-1.60 (m, 2H), 1.58-1.52 (m, 1H), 1.36 (d, J=7.7 Hz, 1H), 1.02 (s, 3H), 0.98 (s, 9H), 0.90 (s, 3H).

### Preparation of Seed Crystals of 3

A solution of 3 (9.2 mg) in ethyl acetate (250 μL) was treated with heptane (150 μL). The resulting solution was concentrated to a solid, which was dissolved in methyl tert-butyl ether (approximately 150 μL) and treated in a drop-wise manner with heptane until a slurry was obtained. This slurry was stirred for 3 days, and subsequently used in the following crystallization.

### Crystallization of 3

The batch of 3 obtained above (128 mg) was combined with a second batch of 3 (approximately 52 mg) in methyl tert-butyl ether (3 mL). After drop-wise addition of heptane (1 mL), the slurry of 3 (9.2 mg) described above was added, and the mixture was stirred at room temperature for 2 days. Filtration provided a filter cake; the flask was rinsed with a mixture of methyl tert-butyl ether and heptane (2:1, 1 mL), which was also filtered. The collected solids afforded (1R,2S,5S)-N-{(2S)-4-(2,4-difluorophenoxy)-3-oxo-1-[(3S)-2-oxopyrrolidin-3-yl]butan-2-yl}-6,6-dimethyl-3-{3-methyl-N-[(trifluoromethyl)sulfonyl]-L-valyl}-3-azabicyclo[3.1.0]hexane-2-carboxamide (3) as a crystalline solid, as judged by analysis under a microscope. Yield: 113 mg, approximately 63% for the crystallization. ¹H NMR (600 MHz, DMSO-d₆) δ 9.64 (d, J=9.1 Hz, 1H), 8.77 (d, J=8.3 Hz, 1H), 7.60 (s, 1H), 7.33-7.25 (m, 1H), 7.08-7.00 (m, 1H), 6.99-6.92 (m, 1H), 5.08 (AB quartet, J_{AB}=18.1 Hz, Δv_{AB}=11.5 Hz, 2H), 4.59-4.49 (m, 1H), 4.29 (s, 1H), 3.93-3.85 (m, 2H), 3.50 (d, J=10.4 Hz, 1H), 3.19-3.11 (m, 1H), 3.10-3.01 (m, 1H), 2.44-2.35 (m, 1H), 2.19-2.10 (m, 1H), 2.00-1.91 (m, 1H), 1.71-1.60 (m, 2H), 1.59-1.52 (m, 1H), 1.36 (d, half of AB quartet, J=7.7 Hz, 1H), 1.03 (s, 3H), 0.98 (s, 9H), 0.90 (s, 3H).

### Example 4

### (1R,2S,5S)-N-{(2S)-4-(2,4-Difluorophenoxy)-3-oxo-1-[(3S)-2-oxopyrrolidin-3-yl]butan-2-yl}-6,6-dimethyl-3-{N-[(trifluoromethyl)sulfonyl]-L-valyl}-3-azabicyclo[3.1.0]hexane-2-carboxamide (4)

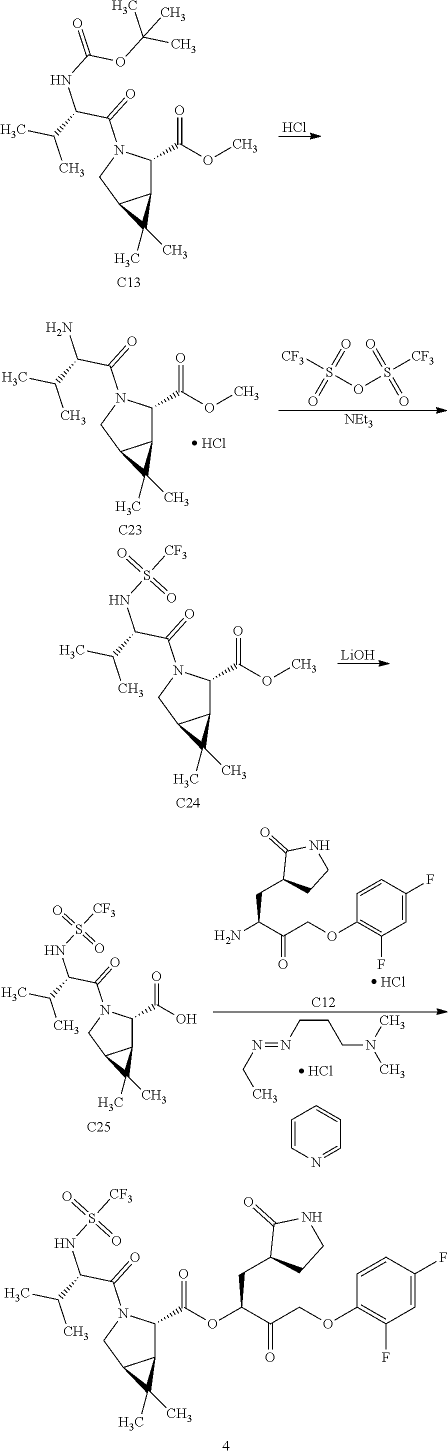

### Step 1. Synthesis of methyl (1R,2S,5S)-6,6-dimethyl-3-L-valyl-3-azabicyclo[3.1.0]hexane-2-carboxylate, hydrochloride salt (C23)

A solution of hydrogen chloride in 1,4-dioxane (4.0 M; 47.5 mL, 190 mmol) was added to a solution of C13 (10.0 g, 27.1 mmol) in dichloromethane (90 mL), and the reaction mixture was allowed to stir overnight at room temperature. After removal of solvents in vacuo, the residue was triturated with diethyl ether to afford C23 as a white solid. Yield: 8.20 g, 26.9 mmol, 99%. LCMS m/z 269.3 [M+H]⁺.

### Step 2. Synthesis of methyl (1R,2S,5S)-6,6-dimethyl-3-{N-[(trifluoromethyl)sulfonyl]-L-valyl}-3-azabicyclo[3.1.0]hexane-2-carboxylate (C24)

A solution of C23 (3.00 g, 9.84 mmol) in dichloromethane (40 mL) was prepared in a flame-dried Schlenk flask. This was cooled to −78° C., treated with triethylamine (2.45 mL, 17.6 mmol), and stirred for 5 minutes. Trifluoromethanesulfonic anhydride (1.41 mL, 8.38 mmol) was then added in a drop-wise manner, and the reaction mixture was stirred at −78° C.; after 30 minutes, conversion to C24 was evidenced via LCMS analysis: LCMS m/z 401.2 [M+H]⁺. When the reaction mixture had been stirred for 1 to 2 hours at −78° C., it was warmed to room temperature and diluted with ice. The aqueous layer was extracted twice with dichloromethane, and the combined organic layers were washed with saturated aqueous sodium chloride solution, dried over sodium sulfate, filtered, and concentrated in vacuo. Silica gel chromatography (Gradient: 0% to 60% ethyl acetate in heptane) afforded C24 as an oil. Yield: 3.20 g, 7.99 mmol, 95%.

### Step 3. Synthesis of (1R,2S,5S)-6,6-dimethyl-3-{N-[(trifluoromethyl)sulfonyl]-L-valyl}-3-azabicyclo[3.1.0]hexane-2-carboxylic acid (C25)

To a solution of C24 (3.20 g, 7.99 mmol) in tetrahydrofuran (20 mL) was added an aqueous solution of lithium hydroxide (2 M; 12 mL, 24 mmol). The reaction mixture was stirred at room temperature for 3 hours, whereupon it was diluted with water and ethyl acetate, and adjusted to pH 12 by addition of 1 M aqueous sodium hydroxide solution. After the resulting mixture had been stirred for 10 minutes, the aqueous layer was acidified to a pH of approximately 2 via addition of 1 M hydrochloric acid. The aqueous layer was then extracted three times with ethyl acetate, and the three organic layers were combined, washed with saturated aqueous sodium chloride solution, dried over magnesium sulfate, filtered, and concentrated in vacuo. The residue was azeotroped once with heptane and twice with a mixture of diethyl ether and heptane, affording C25 as a white/pale-orange solid. Yield: 2.20 g, 5.69 mmol, 71%. LCMS m/z 387.0 [M+H]⁺. ¹H NMR (400 MHz, DMSO-d₆) δ 12.75 (br s, 1H), 9.88 (d, J=8.6 Hz, 1H), 4.15 (s, 1H), 3.85 (dd, J=8.2, 8.2 Hz, 1H), 3.78 (dd, component of ABX system, J=10.4, 5.4 Hz, 1H), 3.59 (d, half of AB quartet, J=10.4 Hz, 1H), 2.08-1.95 (m, 1H), 1.54 (dd, component of ABX system, J=7.6, 5.2 Hz, 1H), 1.44 (d, half of AB quartet, J=7.6 Hz, 1H), 1.02 (s, 3H), 0.99 (d, J=6.8 Hz, 3H), 0.92 (d, J=6.7 Hz, 3H), 0.88 (s, 3H).

Step 4. Synthesis of (1R,2S,5S)-N-{(2S)-4-(2,4-difluorophenoxy)-3-oxo-1-[(3S)-2-oxopyrrolidin-3-yl]butan-2-yl}-6,6-dimethyl-3-{N-[(trifluoromethyl)sulfonyl]-L-valyl}-3-azabicyclo[3.1.0]hexane-2-carboxamide (4)

To a 0° C. solution of C25 (40 mg, 0.10 mmol) and C12 (41.6 mg, 0.124 mmol) in acetonitrile (1 mL) was added 1-[3-(dimethylamino)propyl]-3-ethylcarbodiimide hydrochloride (21.8 mg, 0.114 mmol), followed by drop-wise addition of pyridine (23.4 μL, 0.289 mmol). After the reaction mixture had been stirred at room temperature for 2 hours, it was diluted with ethyl acetate and water. The resulting mixture was washed with 1 M hydrochloric acid, and the aqueous layer was then extracted with ethyl acetate. The combined organic layers were washed with saturated aqueous sodium bicarbonate solution and with saturated aqueous sodium chloride solution, dried over magnesium sulfate, filtered, and concentrated in vacuo. Purification via reversed-phase HPLC (Column: Waters Sunfire C18, 19×100 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95.0% B over 8.54 minutes, then 95.0% B for 1.46 minutes; Flow rate: 25 mL/minute) provided (1R,2S,5S)-N-{(2S)-4-(2,4-difluorophenoxy)-3-oxo-1-[(3S)-2-oxopyrrolidin-3-yl]butan-2-yl}-6,6-dimethyl-3-{N-[(trifluoromethyl)sulfonyl]-L-valyl}-3-azabicyclo[3.1.0]hexane-2-carboxamide (4). Yield: 25.9 mg, 38.8 μmol, 39%. LCMS m/z 667.7 [M+H]⁺. Retention time: 3.10 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B, linear over 4.0 minutes, then 95% B for 1.0 minute; Flow rate: 2 mL/minute).

### Example 5

### (1R,2S,5S)-6,6-Dimethyl-N-{(2S)-3-oxo-1-[(3S)-2-oxopyrrolidin-3-yl]-4-[3-(trifluoromethyl)phenoxy]butan-2-yl}-3-{N-[(trifluoromethyl)sulfonyl]-L-valyl}-3-azabicyclo[3.1.0]hexane-2-carboxamide (5)

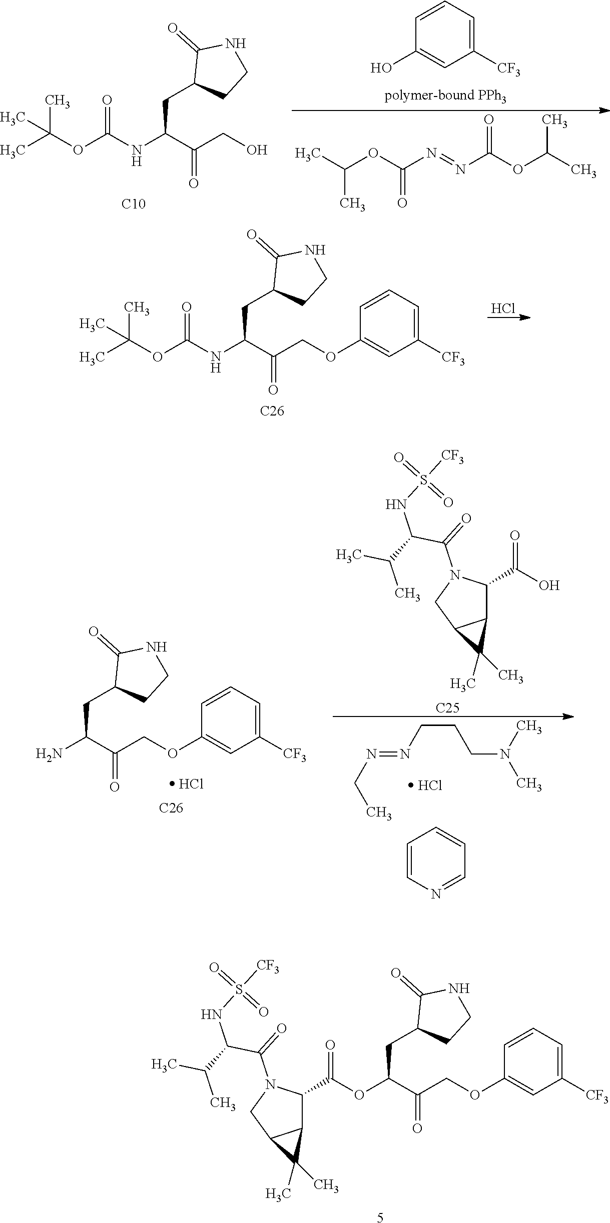

### Step 1. Synthesis of tert-butyl {(2S)-3-oxo-1-[(3S)-2-oxopyrrolidin-3-yl]-4-[3-(trifluoromethyl)phenoxy]butan-2-yl}carbamate (C26)

To a suspension of polymer-bound triphenylphosphine (approximately 3 mmol/g; 1.03 g, 3.09 mmol) in tetrahydrofuran (6 mL) was added a solution of C10 (520 mg, 1.82 mmol) in tetrahydrofuran (2 mL), followed by a solution of 3-(trifluoromethyl)phenol (500 mg, 3.08 mmol) in tetrahydrofuran (1.0 mL). After the reaction mixture had been cooled to 0° C., it was treated with diisopropyl azodicarboxylate (0.535 mL, 2.72 mmol), and stirred for 15 minutes at 0° C. before being warmed to room temperature. After stirring for 1.75 hours at room temperature, the reaction mixture was diluted with dichloromethane and filtered; the filter cake was washed with dichloromethane, and the combined filtrates were concentrated in vacuo. Silica gel chromatography (Gradient: 0% to 10% methanol in dichloromethane) provided material that was then azeotroped with heptane to afford C26 as a white solid. Yield: 328 mg, 0.762 mmol, 42%. LCMS m/z 453.0 [M+Na⁺]. ¹H NMR (400 MHz, DMSO-d₆), characteristic peaks: δ 7.68 (br s, 1H), 7.64 (d, J=7.2 Hz, 1H), 7.50 (br dd, component of ABX system, J=8, 8 Hz, 1H), 7.29 (d, half of AB quartet, J=7.7 Hz, 1H), 7.24-7.17 (m, 2H), 5.11 (s, 2H), 4.18 (ddd, J=10.9, 6.9, 4.2 Hz, 1H), 1.91 (ddd, J=13.8, 11.0, 4.7 Hz, 1H), 1.40 (s, 9H).

### Step 2. Synthesis of (3S)-3-{(2S)-2-amino-3-oxo-4-[3-(trifluoromethyl)phenoxy]butyl}pyrrolidin-2-one, hydrochloride salt (C27)

A solution of C26 (328 mg, 0.762 mmol) in dichloromethane (4 mL) was treated with a solution of hydrogen chloride in 1,4-dioxane (4.0 M; 1.91 mL, 7.64 mmol) and stirred at room temperature. Methanol (1 mL) was added to enhance solubility; after 1.5 hours, LCMS analysis indicated that deprotection to provide C27 was complete: LCMS m/z 331.0 [M+H]⁺. The reaction mixture was concentrated in vacuo, and the residue was azeotroped twice with heptane. The resulting foam was triturated twice with diethyl ether to afford C27 as a pale pink/orange solid (302 mg). A portion of this material was used in the following step. ¹H NMR (400 MHz, DMSO-d₆) δ 8.57 (br s, 3H), 8.02 (s, 1H), 7.55 (dd, component of ABX system, J=8.4, 8.4 Hz, 1H), 7.34 (d, J=8.4 Hz, 1H), 7.33-7.27 (m, 2H), 5.26 (AB quartet, J_{AB}=18.1 Hz, Δv_{AB}=42.4 Hz, 2H), 4.44-4.33 (m, 1H), 3.29-3.16 (m, 2H), 2.71-2.58 (m, 1H), 2.38-2.27 (m, 1H), 2.12-2.02 (m, 1H), 1.93 (ddd, component of ABXY system, J=14.8, 9.8, 6.9 Hz, 1H), 1.83-1.70 (m, 1H).

Step 3. Synthesis of (1R,2S,5S)-6,6-dimethyl-N-{(2S)-3-oxo-1-[(3S)-2-oxopyrrolidin-3-yl]-4-[3-(trifluoromethyl)phenoxy]butan-2-yl}-3-{N-[(trifluoromethyl)sulfonyl]-L-valyl}-3-azabicyclo[3.1.0]hexane-2-carboxamide (5)

To a 0° C. solution of C25 (40 mg, 0.10 mmol) and C27 (from the previous step; 45.6 mg, <0.115 mmol) in acetonitrile (1 mL) was added 1-[3-(dimethylamino)propyl]-3-ethylcarbodiimide hydrochloride (21.8 mg, 0.114 mmol), followed by drop-wise addition of pyridine (23.4 μL, 0.289). After the reaction mixture had been stirred at room temperature for 75 minutes, it was diluted with ethyl acetate and water, then washed with 1 M hydrochloric acid. The aqueous layer was extracted with ethyl acetate, and the combined organic layers were washed with saturated aqueous sodium bicarbonate solution and with saturated aqueous sodium chloride solution, dried over magnesium sulfate, filtered, and concentrated in vacuo. Reversed-phase HPLC (Column: Waters Sunfire C18, 19×100 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95.0% B over 8.54 minutes, then 95.0% B for 1.46 minutes; Flow rate: 25 mL/minute) afforded (1R,2S,5S)-6,6-dimethyl-N-{(2S)-3-oxo-1-[(3S)-2-oxopyrrolidin-3-yl]-4-[3-(trifluoromethyl)phenoxy]butan-2-yl}-3-{N-[(trifluoromethyl) sulfonyl]-L-valyl}-3-azabicyclo[3.1.0]hexane-2-carboxamide (5). Yield: 21.1 mg, 30.2 μmol, 30%. LCMS m/z 699.7 [M+H]⁺. Retention time: 3.27 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B, linear over 4.0 minutes, then 95% B for 1.0 minute; Flow rate: 2 mL/minute).

### Example 6

(1R,2S,5S)-6,6-Dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-{3-methyl-N-[(trifluoromethyl)sulfonyl]-L-valyl}-3-azabicyclo[3.1.0]hexane-2-carboxamide (6)

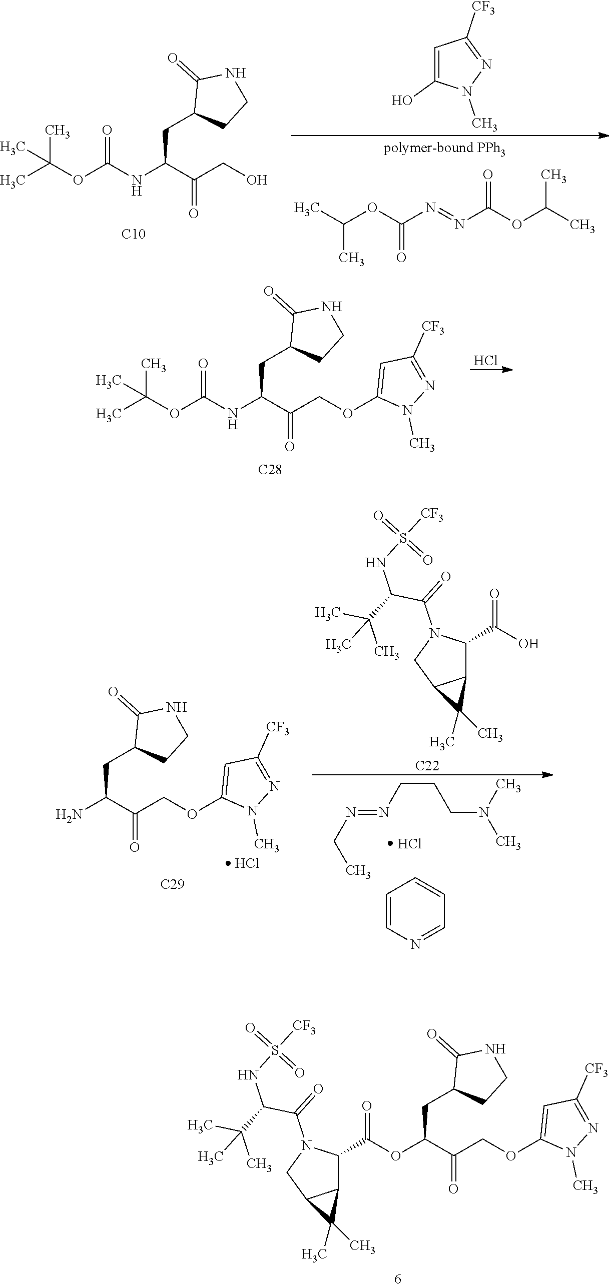

### Step 1. Synthesis of tert-butyl {(2S)-4-{[1-methyl-3-(trifluoromethyl)-1H-pyrazol-5-yl]oxy}-3-oxo-1-[(3S)-2-oxopyrrolidin-3-yl]butan-2-yl}carbamate (C28)

To a suspension of polymer-bound triphenylphosphine (approximately 3 mmol/g; 990 mg, 2.97 mmol) in tetrahydrofuran (6 mL) was added a solution of C10 (500 mg, 1.75 mmol) in tetrahydrofuran (2.7 mL), followed by 1-methyl-3-(trifluoromethyl)-1H-pyrazol-5-ol (493 mg, 2.97 mmol). The resulting mixture was cooled to 0° C., treated with diisopropyl azodicarboxylate (0.514 mL, 2.61 mmol), and stirred at 0° C. for 15 minutes, whereupon it was warmed to room temperature and stirred for 1.5 hours. The reaction mixture was then diluted with dichloromethane and filtered; the filter cake was washed with dichloromethane, and the combined filtrates were concentrated onto silica gel and purified via silica gel chromatography (Gradient: 0% to 10% methanol in dichloromethane). The isolated material was azeotroped first with heptane, and then with a mixture of diethyl ether and heptane, to afford C28 as a white solid. Yield: 367 mg, 0.845 mmol, 48%. LCMS m/z 433.0 [M−H]⁻. ¹H NMR (400 MHz, DMSO-d₆) δ 7.67 (br s, 1H), 7.63 (d, J=7.2 Hz, 1H), 6.09 (s, 1H), 5.15 (AB quartet, J_{AB}=17.8 Hz, Δv_{AB}=12.2 Hz, 2H), 4.15 (ddd, J=10.8, 7.1, 4.2 Hz, 1H), 3.68 (s, 3H), 3.22-3.09 (m, 2H), 2.31-2.21 (m, 1H), 2.21-2.11 (m, 1H), 1.89 (ddd, J=13.7, 11.0, 4.8 Hz, 1H), 1.72-1.57 (m, 2H), 1.39 (s, 9H).

### Step 2. Synthesis of (3S)-3-[(2S)-2-amino-4-{[1-methyl-3-(trifluoromethyl)-1H-pyrazol-5-yl]oxy}-3-oxobutyl]pyrrolidin-2-one, hydrochloride salt (C29)

A solution of C28 (367 mg, 0.845 mmol) in dichloromethane (4 mL) was treated with a solution of hydrogen chloride in 1,4-dioxane (4 M; 2.11 mL, 8.44 mmol) and stirred at room temperature; methanol (1 mL) was added to provide a solution. After 2 hours, LCMS analysis indicated that deprotection to C29 was complete: LCMS m/z 335.0 [M+H]⁺. The reaction mixture was concentrated in vacuo, and the residue was azeotroped with methanol, then with heptane. Trituration of the resulting foam was carried out twice with diethyl ether to provide C29 as a solid (335 mg), a portion of which was taken to the following step. ¹H NMR (400 MHz, DMSO-d₆) δ 8.59 (br s, 3H), 8.03 (s, 1H), 6.26 (s, 1H), 5.33 (AB quartet, J_{AB}=18.0 Hz, Δv_{AB}=58.6 Hz, 2H), 4.41-4.31 (m, 1H), 3.71 (s, 3H), 3.29-3.15 (m, 2H), 2.67-2.56 (m, 1H), 2.37-2.27 (m, 1H), 2.05 (ddd, component of ABXY system, J=14.9, 7.6, 3.3 Hz, 1H), 1.92 (ddd, component of ABXY system, J=14.8, 9.7, 7.0 Hz, 1H), 1.82-1.68 (m, 1H).

Step 3. Synthesis of (1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-{3-methyl-N-[(trifluoromethyl)sulfonyl]-L-valyl}-3-azabicyclo[3.1.0]hexane-2-carboxamide (6)

A 0° C. solution of C22 (125 mg, 0.312 mmol) and C29 (from the previous step; 135 mg, ≤0.340 mmol) in acetonitrile (3.1 mL) was treated with 1-[3-(dimethylamino)propyl]-3-ethylcarbodiimide hydrochloride (65.8 mg, 0.343 mmol), followed by drop-wise addition of pyridine (70.7 μL, 0.874 mmol). After the reaction mixture had been stirred at room temperature for 2 hours, a solution of C29 (30 mg, ≤0.11 mmol) in acetonitrile was added. After an additional 1.5 hours, a solution of C29 (25 mg, 563 μmol) in acetonitrile was again added, and stirring was continued for 1.5 hours, whereupon the reaction mixture was diluted with ethyl acetate and water. The resulting mixture was washed with 1 M hydrochloric acid, and the aqueous layer was extracted with ethyl acetate. The combined organic layers were washed with saturated aqueous sodium bicarbonate solution and with saturated aqueous sodium chloride solution, dried over magnesium sulfate, filtered, and concentrated in vacuo. Silica gel chromatography (Gradient: 0% to 10% methanol in dichloromethane) provided a colorless glass, which was azeotroped twice with heptane and once with a mixture of diethyl ether and heptane, affording (1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-{3-methyl-N-[(trifluoromethyl)sulfonyl]-L-valyl}-3-azabicyclo[3.1.0]hexane-2-carboxamide (6) as a white solid. Yield: 122 mg, 0.170 mmol, 54%. LCMS m/z 717.2 [M+H]⁺. ¹H NMR (400 MHz, DMSO-d₆) δ 9.65 (d, J=9.1 Hz, 1H), 8.79 (d, J=8.3 Hz, 1H), 7.60 (s, 1H), 6.09 (s, 1H), 5.15 (s, 2H), 4.55-4.46 (m, 1H), 4.27 (s, 1H), 3.94-3.85 (m, 2H), 3.67 (s, 3H), 3.51 (d, J=10.3 Hz, 1H), 3.19-3.11 (m, 1H), 3.10-3.01 (m, 1H), 2.43-2.31 (m, 1H), 2.20-2.09 (m, 1H), 2.03-1.91 (m, 1H), 1.70-1.57 (m, 2H), 1.54 (br dd, J=7, 6 Hz, 1H), 1.34 (d, half of AB quartet, J=7.7 Hz, 1H), 1.01 (s, 3H), 0.99 (s, 9H), 0.90 (s, 3H).

### Example 7

(1R,2S,5S)-6,6-Dimethyl-3-[3-methyl-N-(methylsulfonyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (7)

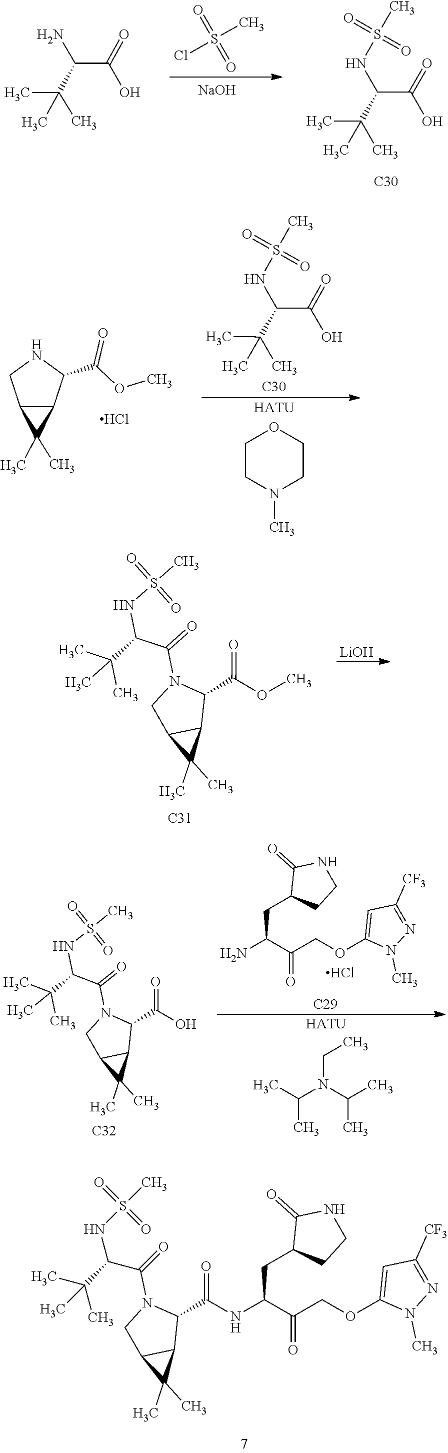

### Step 1. Synthesis of 3-methyl-N-(methylsulfonyl)-L-valine (C30)

To a 0° C. solution of 3-methylvaline (13.0 g, 99.1 mmol) in a mixture of water (150 mL) and tetrahydrofuran (100 mL) was added a solution of methanesulfonyl chloride (14.2 g, 124 mmol) in tetrahydrofuran (50 mL) and an aqueous solution of sodium hydroxide (1 M; 223 mmol, 223 mL). After the reaction mixture had warmed to room temperature and stirred for 16 hours, it was adjusted to a pH of approximately 1 by addition of 1 M hydrochloric acid and then extracted with ethyl acetate (3×100 mL). The combined organic layers were dried over sodium sulfate, filtered, and concentrated in vacuo; this material was combined with the product from a similar reaction carried out using 3-methylvaline (12.0 g, 91.5 mmol) and concentrated under reduced pressure to afford C30 as a white solid. Combined yield: 15.0 g, 71.7 mmol, 38%. LCMS m/z 208.1 [M−H]⁻. ¹H NMR (400 MHz, DMSO-d₆) δ 7.36 (d, J=9.9 Hz, 1H), 3.55 (d, J=9.9 Hz, 1H), 2.85 (s, 3H), 0.95 (s, 9H).

### Step 2. Synthesis of methyl (1R,2S,5S)-6,6-dimethyl-3-[3-methyl-N-(methylsulfonyl)-L-valyl]-3-azabicyclo[3.1.0]hexane-2-carboxylate (C31)

To a 0° C. solution of methyl (1R,2S,5S)-6,6-dimethyl-3-azabicyclo[3.1.0]hexane-2-carboxylate, hydrochloride salt (500 mg, 2.43 mmol) and C30 (534 mg, 2.55 mmol) in N,N-dimethylformamide (10 mL) was added O-(7-azabenzotriazol-1-yl)-N,N,N′N′-tetramethyluronium hexafluorophosphate (HATU; 1.20 g, 3.16 mmol) in one portion, followed by drop-wise addition of 4-methylmorpholine (1.23 g, 12.2 mmol). After the reaction mixture had been stirred at 0° C. for 10 minutes, it was warmed to room temperature (27° C.) and allowed to stir for 18 hours, whereupon it was poured into ice water (40 mL) and extracted with ethyl acetate (3×40 mL). The combined organic layers were washed sequentially with water (40 mL), hydrochloric acid (1 M; 30 mL), aqueous sodium carbonate solution (˜10%; 2×40 mL), and saturated aqueous sodium chloride solution, dried over sodium sulfate, filtered, and concentrated in vacuo. Chromatography on silica gel (Gradient: 20% to 50% ethyl acetate in petroleum ether) provided C31 as a colorless gum. Yield: 560 mg, 1.55 mmol, 64%. ¹H NMR (400 MHz, DMSO-d₆) δ 7.11 (d, J=9.5 Hz, 1H), 4.22 (s, 1H), 3.86-3.78 (m, 2H), 3.71 (d, half of AB quartet, J=10.4 Hz, 1H), 3.65 (s, 3H), 2.85 (s, 3H), 1.56 (dd, component of ABX system, J=7.6, 5.3 Hz, 1H), 1.43 (d, half of AB quartet, J=7.6 Hz, 1H), 1.01 (s, 3H), 0.97 (s, 9H), 0.90 (s, 3H).

### Step 3. Synthesis of (1R,2S,5S)-6,6-dimethyl-3-[3-methyl-N-(methylsulfonyl)-L-valyl]-3-azabicyclo[3.1.0]hexane-2-carboxylic acid (C32)

To a 5° C. solution of C31 (550 mg, 1.53 mmol) in a mixture of methanol (6 mL), tetrahydrofuran (6 mL), and water (6 mL) was added lithium hydroxide monohydrate (141 mg, 3.36 mmol). The reaction mixture was then stirred at room temperature (25° C. to 30° C.) for 18 hours, whereupon the organic solvents were removed via concentration in vacuo. The aqueous residue was diluted with water (5 mL) and extracted with ethyl acetate (20 mL). This organic layer was discarded; the aqueous layer was adjusted to pH 1 to 2 by addition of concentrated hydrochloric acid and extracted with ethyl acetate (3×30 mL). These organic layers were combined, dried over sodium sulfate, filtered, and concentrated in vacuo to afford C32 as a white solid. Yield: 503 mg, 1.45 mmol, 95%. ¹H NMR (400 MHz, DMSO-d₆) δ 7.07 (d, J=9.5 Hz, 1H), 4.13 (s, 1H), 3.85-3.76 (m, 2H), 3.69 (d, half of AB quartet, J=10.5 Hz, 1H), 2.84 (s, 3H), 1.53 (dd, component of ABX system, J=7.6, 5.3 Hz, 1H), 1.40 (d, half of AB quartet, J=7.6 Hz, 1H), 1.01 (s, 3H), 0.97 (s, 9H), 0.89 (s, 3H).

Step 4. Synthesis of (1R,2S,5S)-6,6-dimethyl-3-[3-methyl-N-(methylsulfonyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (7)

To a 0° C. solution of C29 (90 mg, 0.24 mmol) and C32 (101 mg, 0.292 mmol) in N,N-dimethylformamide (3 mL) was added O-(7-azabenzotriazol-1-yl)-N,N,N′N′-tetramethyluronium hexafluorophosphate (HATU; 102 mg, 0.268 mmol) in one portion, followed by drop-wise addition of N,N-diisopropylethylamine (110 mg, 0.851 mmol). The reaction mixture was stirred at 0° C. for 10 minutes, then at room temperature for 2 hours, whereupon it was poured into ice water (30 mL) and extracted with ethyl acetate (3×30 mL). The combined organic layers were washed sequentially with water (20 mL), hydrochloric acid (1 M; 20 mL), saturated aqueous sodium bicarbonate solution (20 mL), and saturated aqueous sodium chloride solution, dried over sodium sulfate, filtered, and concentrated in vacuo. Chromatography on silica gel (Gradient: 20% to 100% ethyl acetate in petroleum ether) was followed by supercritical fluid chromatography [Column: Chiral Technologies Chiralpak AS, 30×250 mm, 10 μm; Mobile phase: 7:3 carbon dioxide/(ethanol containing 0.1% ammonium hydroxide), Flow rate: 60 mL/minute] to afford (1R,2S,5S)-6,6-dimethyl-3-[3-methyl-N-(methylsulfonyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (7) as a white solid. Yield: 12.8 mg, 19.3 μmol, 8%. LCMS m/z 663.3 [M+H]⁺. ¹H NMR (400 MHz, DMSO-d₆), 75.4° C.: δ 8.60 (d, J=7.9 Hz, 1H), 7.36 (s, 1H), 6.74 (br d, J=9.3 Hz, 1H), 6.04 (s, 1H), 5.11 (AB quartet, J_{AB}=17.6 Hz, Δv_{AB}=7.2 Hz, 2H), 4.50 (ddd, J=11.4, 7.8, 3.9 Hz, 1H), 4.30 (s, 1H), 3.90-3.79 (m, 2H), 3.75-3.65 (m, 1H), 3.69 (s, 3H), 3.21-3.14 (m, 1H), 3.14-3.06 (m, 1H, assumed; partially obscured by water peak), 2.86 (s, 3H), 2.46-2.35 (m, 1H), 2.23-2.13 (m, 1H), 2.01 (ddd, J=13.9, 11.2, 4.3 Hz, 1H), 1.74-1.62 (m, 2H), 1.52 (dd, J=7.7, 5.4 Hz, 1H), 1.31 (d, half of AB quartet, J=7.7 Hz, 1H), 1.03 (s, 3H), 0.98 (s, 9H), 0.94 (s, 3H).

### Example 8

(1R,2S,5S)-6,6-Dimethyl-3-[3-methyl-N-(trifluoroacetyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (8)

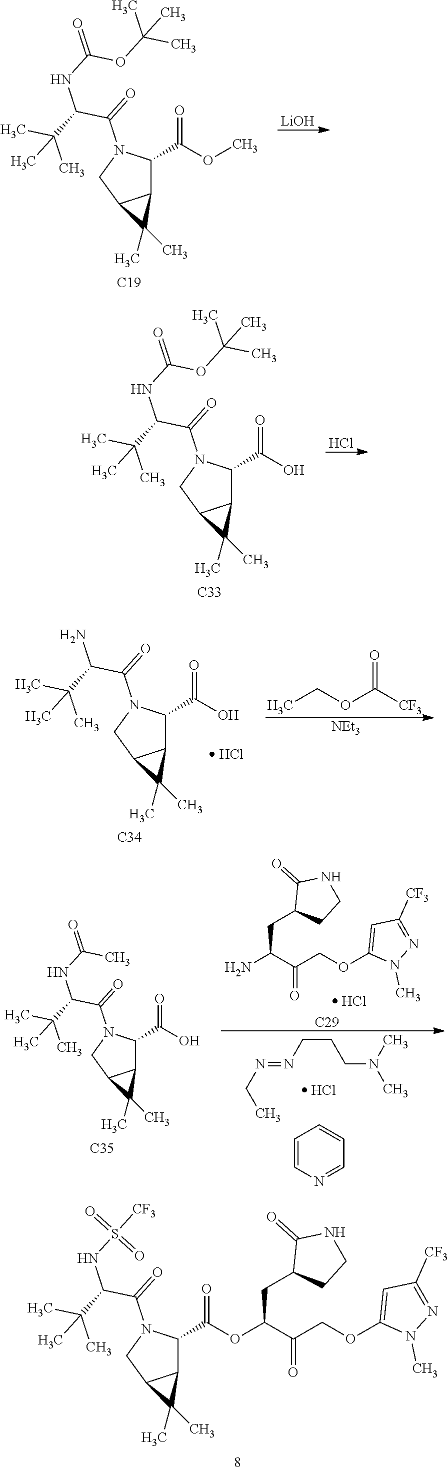

### Step 1. Synthesis of (1R,2S,5S)-3-[N-(tert-butoxycarbonyl)-3-methyl-L-valyl]-6,6-dimethyl-3-azabicyclo[3.1.0]hexane-2-carboxylic acid (C33)

An aqueous solution of lithium hydroxide (230 mL, 692 mmol) was added to a solution of C19 (85 g, 220 mmol) in tetrahydrofuran (230 mL). The reaction mixture was stirred at room temperature for 2 hours, whereupon LCMS analysis indicated complete conversion to C33: LCMS m/z 369.3 [M+H]⁺. After the tetrahydrofuran had been removed via concentration in vacuo, the aqueous residue was diluted with water (250 mL) and hydrochloric acid (1 M; 250 mL) and stirred for 2 minutes. The resulting mixture was acidified to a pH of approximately 2 by addition of concentrated hydrochloric acid, then diluted with ethyl acetate (250 mL). The aqueous layer was further extracted with ethyl acetate (2×150 mL), and the combined organic layers were washed with saturated aqueous sodium chloride solution, dried over sodium sulfate, filtered, and concentrated in vacuo, affording C33 as a solid (85 g). The bulk of this material was used in the following step. ¹H NMR (400 MHz, DMSO-d₆) δ 12.55 (br s, 1H), 6.67 (d, J=9.4 Hz, 1H), 4.13 (s, 1H), 4.05 (d, J=9.5 Hz, 1H), 3.91 (d, half of AB quartet, J=10.4 Hz, 1H), 3.77 (dd, component of ABX system, J=10.3, 5.3 Hz, 1H), 1.50 (dd, J=7.6, 5.1 Hz, 1H), 1.39 (d, J=7.6 Hz, 1H), 1.35 (s, 9H), 1.01 (s, 3H), 0.93 (s, 9H), 0.84 (s, 3H).

### Step 2. Synthesis of (1R,2S,5S)-6,6-dimethyl-3-(3-methyl-L-valyl)-3-azabicyclo[3.1.0]hexane-2-carboxylic acid, hydrochloride salt (C34)

To a solution of C33 (from the previous step; 81 g, 5210 mmol) in dichloromethane (220 mL) was added a solution of hydrogen chloride in 1,4-dioxane (4 M; 275 mL, 1.10 mol). After the reaction mixture had been stirred at room temperature for 18 hours, it was concentrated in vacuo to provide C34 as a white solid (66.5 g). The bulk of this material was used in the following step. LCMS m/z 269.3 [M+H]⁺. ¹H NMR (400 MHz, DMSO-d₆) δ 12.80 (v br s, 1H), 8.18 (br s, 3H), 4.18 (s, 1H), 3.86-3.75 (m, 2H), 3.71 (d, half of AB quartet, J=10.8 Hz, 1H), 1.57 (dd, component of ABX system J=7.7, 5.3 Hz, 1H), 1.46 (d, half of AB quartet, J=7.7 Hz, 1H), 1.05-1.01 (m, 12H), 0.96 (s, 3H).

### Step 3. Synthesis of (1R,2S,5S)-6,6-dimethyl-3-[3-methyl-N-(trifluoroacetyl)-L-valyl]-3-azabicyclo[3.1.0]hexane-2-carboxylic acid (C35)

A solution of C34 (from the previous step; 55.0 g, 5174 mmol) in methanol (180 mL) was cooled to 0° C. and treated with triethylamine (151 mL, 1.08 mol). After the reaction mixture had warmed to room temperature, it was stirred for 5 minutes, whereupon ethyl trifluoroacetate (53.7 mL, 451 mmol) was gradually added. The reaction mixture was heated at 50° C. for 18 hours, at which time LCMS analysis indicated complete conversion to C35: LCMS m/z 365.1 [M+H]⁺. It was then concentrated in vacuo, and the residue was mixed with water (250 mL) and hydrochloric acid (1 M; 250 mL) and stirred for 2 minutes. Concentrated hydrochloric acid was then used to adjust the pH to 2. After an initial extraction with ethyl acetate (250 mL), the aqueous layer was further extracted with ethyl acetate (2×150 mL); the combined organic layers were washed with saturated aqueous sodium chloride solution, dried over sodium sulfate, filtered, and concentrated in vacuo, affording C35 as a solid. Yield: 70 g, assumed quantitative. ¹H NMR (400 MHz, DMSO-d₆) δ 12.74 (v br s, 1H), 9.43 (d, J=8.5 Hz, 1H), 4.44 (d, J=8.5 Hz, 1H), 4.15 (s, 1H), 3.85 (dd, component of ABX system, J=10.5, 5.3 Hz, 1H), 3.72 (d, half of AB quartet, J=10.5 Hz, 1H), 1.53 (dd, component of ABX system, J=7.6, 5.1 Hz, 1H), 1.43 (d, half of AB quartet, J=7.6 Hz, 1H), 1.03-0.98 (m, 12H), 0.82 (s, 3H).

Step 4. Synthesis of (1R,2S,5S)-6,6-dimethyl-3-[3-methyl-N-(trifluoroacetyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (8)

To a 0° C. solution of C35 (58 mg, 0.16 mmol) and C29 (62.6 mg, 0.169 mmol) in acetonitrile (1.6 mL) was added 1-[3-(dimethylamino)propyl]-3-ethylcarbodiimide hydrochloride (EDCl; 33.6 mg, 0.175 mmol), followed by drop-wise addition of pyridine (36.0 μL, 0.445 mmol). After the reaction mixture had been stirred at room temperature for 30 minutes, LCMS analysis indicated the presence of 8: LCMS m/z 681.3 [M+H]*, and the reaction mixture was diluted with ethyl acetate and water, then washed with 1 M hydrochloric acid. The aqueous layer was extracted with ethyl acetate, and the combined organic layers were washed sequentially with saturated aqueous sodium bicarbonate solution and saturated aqueous sodium chloride solution, dried over magnesium sulfate, filtered, and concentrated in vacuo. The residue was taken up in dichloromethane and concentrated onto silica gel; silica gel chromatography (Gradient 0% to 10% methanol in dichloromethane) provided (1R,2S,5S)-6,6-dimethyl-3-[3-methyl-N-(trifluoroacetyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide (8) as a solid. Yield: 23.7 mg, 34.8 μmol, 22%. ¹H NMR (400 MHz, methanol-d₄) δ 8.93 (d, J=7.5 Hz, <1H; incompletely exchanged), 5.99 (s, 1H), 5.11 (s, 2H), 4.68-4.50 (m, 2H), 4.33 (s, 1H), 4.03 (dd, component of ABX system, J=10.3, 5.5 Hz, 1H), 3.85 (d, half of AB quartet, J=10.3 Hz, 1H), 3.74 (s, 3H), 3.40-3.24 (m, 2H, assumed; largely obscured by solvent peak), 2.70-2.58 (m, 1H), 2.41-2.29 (m, 1H), 2.11 (ddd, J=14.0, 11.4, 4.5 Hz, 1H), 1.96-1.76 (m, 2H), 1.59 (dd, J=7.7, 5.4 Hz, 1H), 1.40 (d, half of AB quartet, J=7.6 Hz, 1H), 1.08-1.03 (m, 12H), 0.94 (s, 3H).

TABLE 1
Method of synthesis, structure, and physicochemical data for Examples 9 - 13.

| | | | ¹H NMR (400 MHz, |
|---|---|---|---|
| | | | chloroform-d) δ; Mass |
| | Method of | | spectrum, observed ion |
| | synthesis; | | m/z [M + H]⁺ or HPLC |
| | Non- | | retention time; Mass |
| | commercial | | spectrum m/z |
| Exam- | starting | | [M + H]⁺ (unless |
| ple | materials | Structure | otherwisei ndicated) |
| 9 | Example 5¹; C28, C16 | 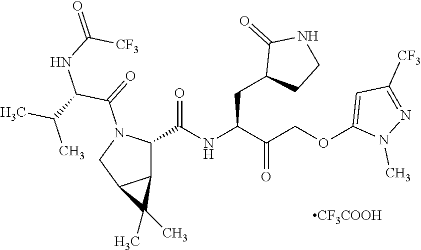 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cc(C(F)(F)F)nn1C)C2(C)C ; "·CF3COOH"] | 2.88 minutes²; 667.6 |
| 10 | Example 5; C22, C27 | 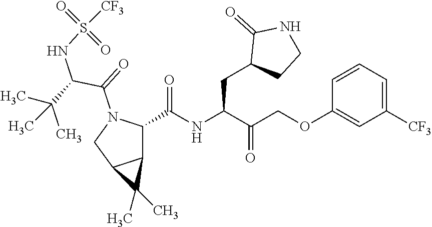 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1cccc(C(F)(F)F)c1)C2(C)C] | 3.39 minutes²; 713.7 |
| 11 | Example 5; C29, C25 | 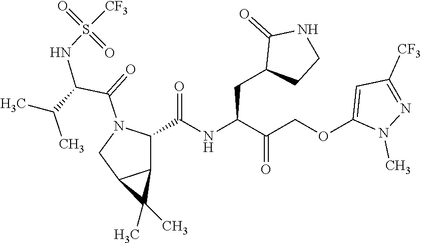 [SMILES: CC(C)[C@H](NS(=O)(=O)C(F)(F)F)C(=O)N1C[C@H]2[C@@H]([C@H]1C(=O)N[C@@H](C[C@@H]1CCNC1=O)C(=O)COc1cc(C(F)(F)F)nn1C)C2(C)C] | 3.03 minutes²; 703.7 |
| 12 | C27, C35³ | 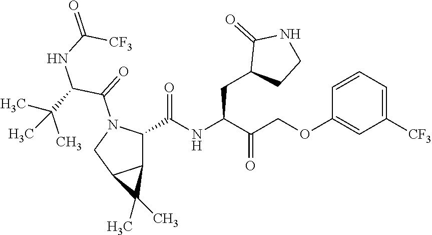 [SMILES: CC(C)(C)[C@H](NC(=O)C(F)(F)F)C(=O)N1C[C@H]2[C@@H]([C@H]1C(=O)N[C@@H](C[C@@H]1CCNC1=O)C(=O)COc1cccc(C(F)(F)F)c1)C2(C)C] | ¹H NMR (400 MHz, DMSO- d₆) δ 9.38 (d, J = 8.3 Hz, 1H), 8.79 (d, J = 7.9 Hz, 1H), 7.63 (s, 1H), 7.50 (br dd, J = 8, 8 Hz, 1H), 7.29 (d, J = 7.6 Hz, 1H), 7.22-7.16 (m, 2H), 5.11 (s, 2H), 4.56-4.48 (m, 1H), 4.42 (d, J = 8.5 Hz, 1H), 4.25 (s, 1H), 3.95-3.86 (m, 1H), 3.69 (d, J = 10.3 Hz, 1H), 3.12-3.02 (m, 1H), 2.22-2.10 (m, 1H), 2.03-1.93 (m, 1H), 1.70-1.59 (m, 2H), 1.54-1.48 (m, 1H), 1.31 (d, half of AB |
| | | | quartet, J = 7.6 Hz, 1H), 1.00 |
| | | | (s, 3H), 0.96 (s, 9H), 0.86 (s, |
| | | | 3H); 699.1 [M + Na⁺] |
| 13 | Example 7; C12, C32 | 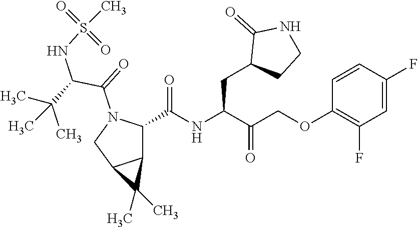 [SMILES: CC(C)(C)[C@H](NS(C)(=O)=O)C(=O)N1C[C@H]2[C@@H]([C@H]1C(=O)N[C@@H](C[C@@H]1CCNC1=O)C(=O)COc1ccc(F)cc1F)C2(C)C] | ¹H NMR (400 MHz, DMSO- d₆) δ 8.77 (d, J = 8.2 Hz, 1H), 7.60 (s, 1H), 7.29 (ddd, J = 11.6, 8.8, 3.0 Hz, 1H), 7.11 (d, J = 9.6 Hz, 1H), 7.04 (ddd, J = 9.4, 9.3, 5.3 Hz, 1H), 7.00-6.92 (m, 1H), 5.08 (AB quartet, J_{AB} = 18.0 Hz, Δ_{VAB} = 12.4 Hz, 2H), 4.54-4.45 (m, 1H), 4.26 (s, 1H), 3.85 (dd, component of ABX system, J = 10.4, 5.7 Hz, 1H), 3.81 (d, J = 9.6 Hz, 1H), 3.68 (d, half of AB quartet, J = 10.4 |
| | | | Hz, 1H), 3.19-3.10 (m, 1H), |
| | | | 3.10-3.01 (m, 1H), 2.83 (s, |
| | | | 3H), 2.45-2.34 (m, 1H), 2.20- |
| | | | 2.10 (m, 1H), 2.02-1.91 (m, |
| | | | 1H), 1.71-1.59 (m, 2H), 1.54 |
| | | | (dd, J = 7.7, 5.4 Hz, 1H), 1.33 |
| | | | (d, half of AB quartet, J = 7.7 |
| | | | Hz, 1H), 1.02 (s, 3H), 0.94 (s, |
| | | | 9H), 0.93 (s, 3H); 627.3 |

1. Treatment of C28 with trifluoroacetic acid provided the trifluoroacetate salt of C29, which was used in the synthesis of Example 9.

2. Conditions for analytical HPLC. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B, linear over 4.0 minutes, then 95% B for 1.0 minute; Flow rate: 2 mL/minute.

3. The coupling between C27 and C35 was mediated via O-(7-azabenzotriazol-1-yl)-N,N,N′,N′-tetramethyluronium hexafluorophosphate (HATU) and 4-methyl morpholine in N,N-dimethylformamide. After the reaction mixture had been poured into ice water, the resulting solid was collected via filtration, washed with water, dissolved in dichloromethane, dried over sodium sulfate, filtered, and concentrated in vacuo. Silica gel chromatography (Gradient: 0% to 100% ethyl acetate in petroleum ether), followed by supercritical fluid chromatography [Column: Chiral Technologies Chiralpak AD, 30×250 mm, 10 μm; Mobile phase: 4:1 carbon dioxide/I (ethanol containing 0.1% ammonium hydroxide); Flow rate: 60 mL/minute] provided Example 12 as a white solid.

Examples 14-113, provided in the table below, can be prepared according to the general methods described herein and in a manner analogous to that as described for Examples 1-13 above.

| Example | Structure |
|---|---|
| 14 | 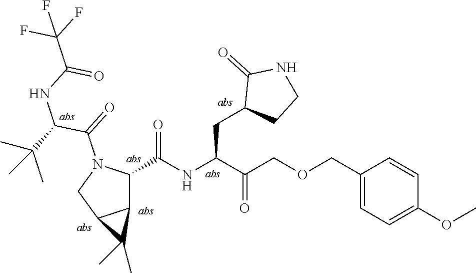 [SMILES: COc1ccc(COCC(=O)[C@H](C[C@@H]2CCNC2=O)NC(=O)[C@@H]2[C@@H]3[C@H](CN2C(=O)[C@@H](NC(=O)C(F)(F)F)C(C)(C)C)C3(C)C)cc1] |
| 15 | 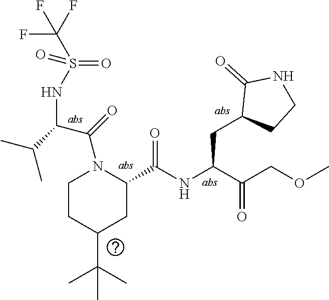 [SMILES: COCC(=O)[C@H](C[C@@H]1CCNC1=O)NC(=O)[C@@H]1CC(C(C)(C)C)CCN1C(=O)[C@@H](NS(=O)(=O)C(F)(F)F)C(C)C] |
| 16 | 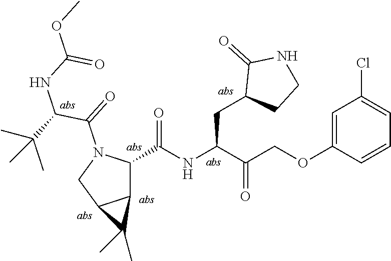 [SMILES: COC(=O)N[C@H](C(=O)N1C[C@H]2[C@@H]([C@H]1C(=O)N[C@@H](C[C@@H]1CCNC1=O)C(=O)COc1cccc(Cl)c1)C2(C)C)C(C)(C)C] |
| 17 | 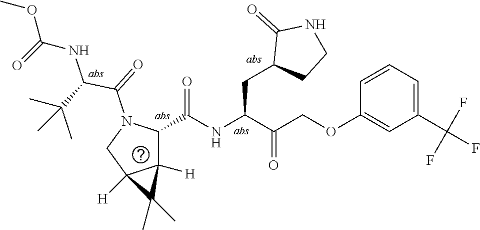 [SMILES: COC(=O)N[C@H](C(=O)N1C[C@H]2[C@@H]([C@H]1C(=O)N[C@@H](C[C@@H]1CCNC1=O)C(=O)COc1cccc(C(F)(F)F)c1)C2(C)C)C(C)(C)C] |
| 18 | 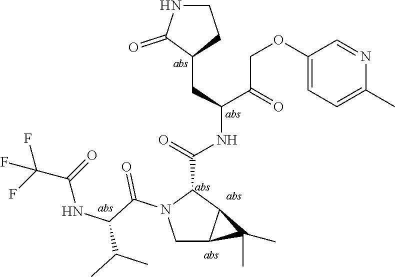 [SMILES: Cc1ccc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)cn1] |
| 19 | 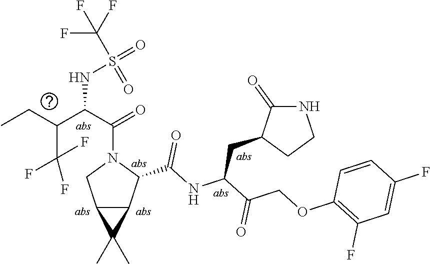 [SMILES: CCC([C@H](NS(=O)(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C)C(F)(F)F] |
| 20 | 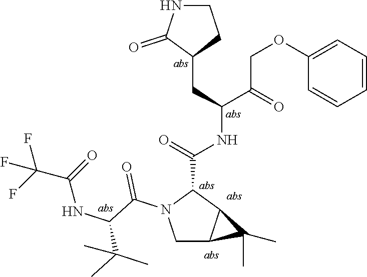 [SMILES: CC(C)(C)[C@H](NC(=O)C(F)(F)F)C(=O)N1C[C@H]2[C@@H]([C@H]1C(=O)N[C@@H](C[C@@H]1CCNC1=O)C(=O)COc1ccccc1)C2(C)C] |
| 21 | |
| 22 | |
| 23 | 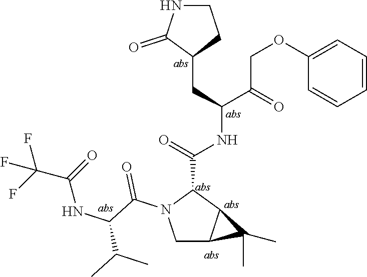 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ccccc1)C2(C)C] |
| 24 | 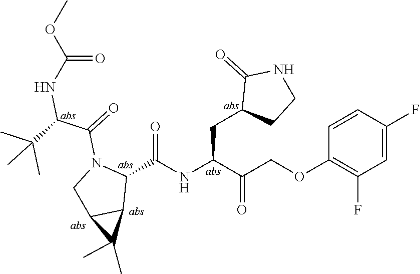 [SMILES: COC(=O)N[C@H](C(=O)N1C[C@H]2[C@@H]([C@H]1C(=O)N[C@@H](C[C@@H]1CCNC1=O)C(=O)COc1ccc(F)cc1F)C2(C)C)C(C)(C)C] |
| 25 | 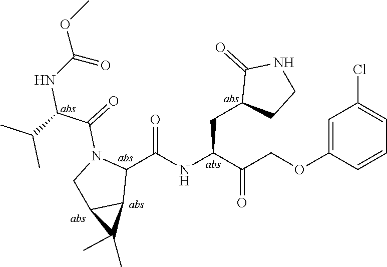 [SMILES: COC(=O)N[C@H](C(=O)N1C[C@H]2[C@@H](C1C(=O)N[C@@H](C[C@@H]1CCNC1=O)C(=O)COc1cccc(Cl)c1)C2(C)C)C(C)C] |
| 26 | 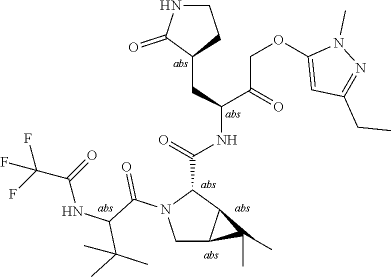 [SMILES: CCc1cc(OCC(=O)[C@H](C[C@@H]2CCNC2=O)NC(=O)[C@@H]2[C@@H]3[C@H](CN2C(=O)C(NC(=O)C(F)(F)F)C(C)(C)C)C3(C)C)n(C)n1] |
| 27 | 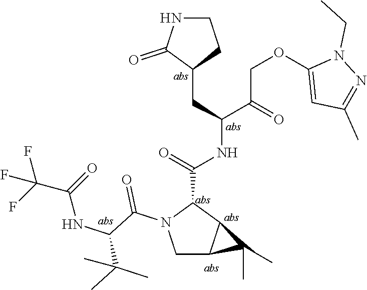 [SMILES: CCn1nc(C)cc1OCC(=O)[C@H](C[C@@H]1CCNC1=O)NC(=O)[C@@H]1[C@@H]2[C@H](CN1C(=O)[C@@H](NC(=O)C(F)(F)F)C(C)(C)C)C2(C)C] |
| 28 | 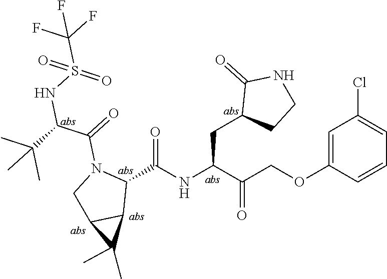 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1cccc(Cl)c1)C2(C)C] |
| 29 | |
| 30 | 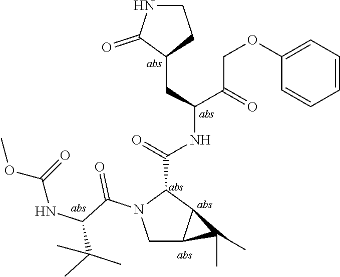 [SMILES: COC(=O)N[C@H](C(=O)N1C[C@H]2[C@@H]([C@H]1C(=O)N[C@@H](C[C@@H]1CCNC1=O)C(=O)COc1ccccc1)C2(C)C)C(C)(C)C] |
| 31 | 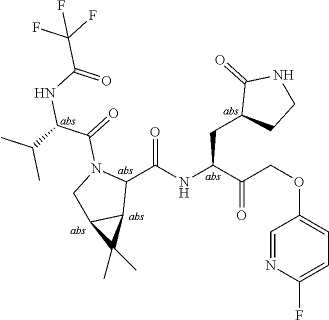 [SMILES: CC(C)[C@H](NC(=O)C(F)(F)F)C(=O)N1C[C@H]2[C@@H](C1C(=O)N[C@@H](C[C@@H]1CCNC1=O)C(=O)COc1ccc(F)nc1)C2(C)C] |
| 32 | 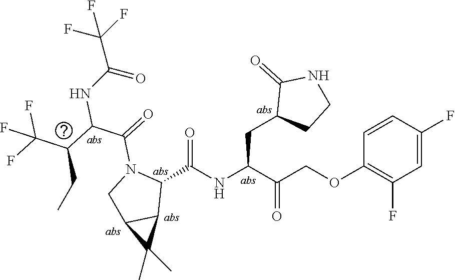 [SMILES: CC[C@H](C(NC(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C)C(F)(F)F] |
| 33 | 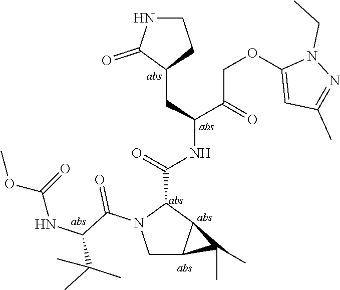 [SMILES: CCn1nc(C)cc1OCC(=O)[C@H](C[C@@H]1CCNC1=O)NC(=O)[C@@H]1[C@@H]2[C@H](CN1C(=O)[C@@H](NC(=O)OC)C(C)(C)C)C2(C)C] |
| 34 | |
| 35 | 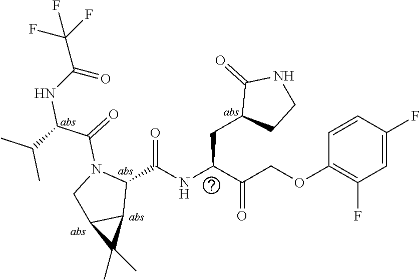 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C] |
| 36 | 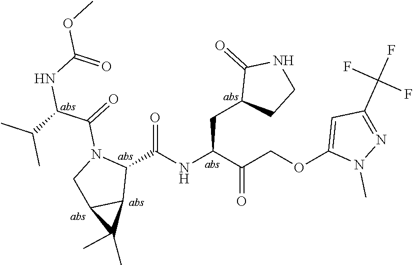 [SMILES: COC(=O)N[C@H](C(=O)N1C[C@H]2[C@@H]([C@H]1C(=O)N[C@@H](C[C@@H]1CCNC1=O)C(=O)COc1cc(C(F)(F)F)nn1C)C2(C)C)C(C)C] |
| 37 | 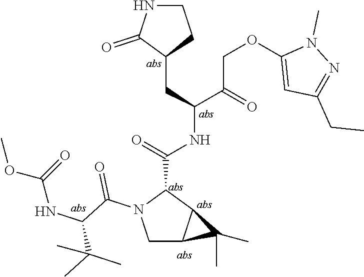 [SMILES: CCc1cc(OCC(=O)[C@H](C[C@@H]2CCNC2=O)NC(=O)[C@@H]2[C@@H]3[C@H](CN2C(=O)[C@@H](NC(=O)OC)C(C)(C)C)C3(C)C)n(C)n1] |
| 38 | 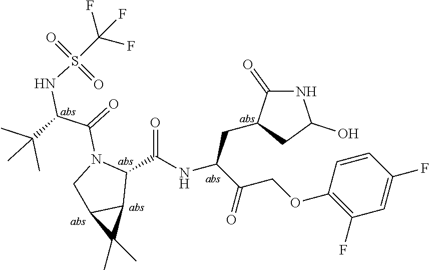 |
| 39 | 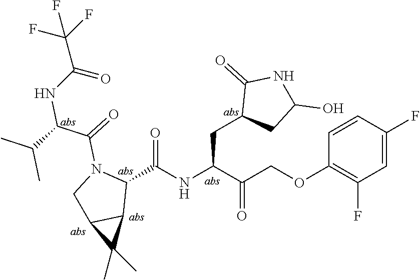 |
| 40 | 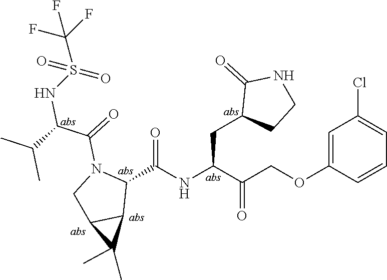 [SMILES: CC(C)[C@H](NS(=O)(=O)C(F)(F)F)C(=O)N1C[C@H]2[C@@H]([C@H]1C(=O)N[C@@H](C[C@@H]1CCNC1=O)C(=O)COc1cccc(Cl)c1)C2(C)C] |
| 41 | 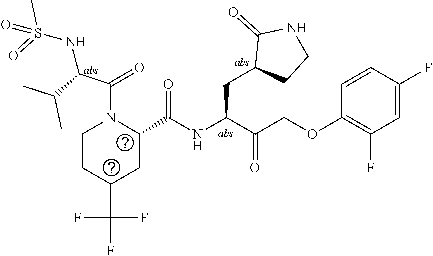 [SMILES: CC(C)[C@H](NS(C)(=O)=O)C(=O)N1CCC(C(F)(F)F)C[C@H]1C(=O)N[C@@H](C[C@@H]1CCNC1=O)C(=O)COc1ccc(F)cc1F] |
| 42 | 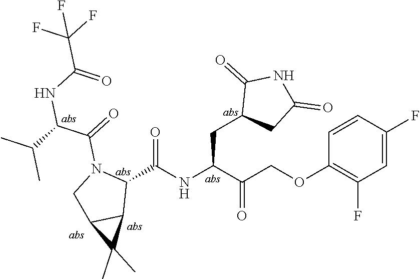 [SMILES: CC(C)[C@H](NC(=O)C(F)(F)F)C(=O)N1C[C@H]2[C@@H]([C@H]1C(=O)N[C@@H](C[C@@H]1CC(=O)NC1=O)C(=O)COc1ccc(F)cc1F)C2(C)C] |
| 43 | 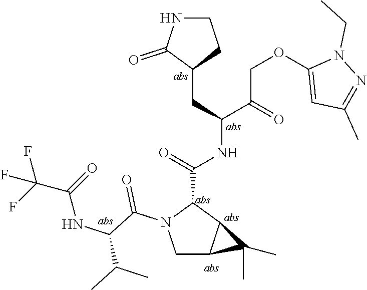 [SMILES: CCn1nc(C)cc1OCC(=O)[C@H](C[C@@H]1CCNC1=O)NC(=O)[C@@H]1[C@@H]2[C@H](CN1C(=O)[C@@H](NC(=O)C(F)(F)F)C(C)C)C2(C)C] |
| 44 | 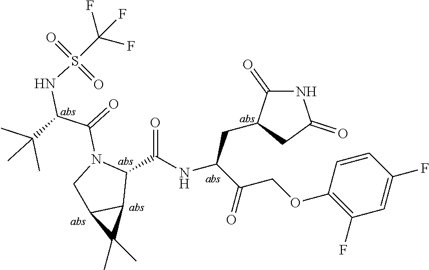 [SMILES: CC(C)(C)[C@H](NS(=O)(=O)C(F)(F)F)C(=O)N1C[C@H]2[C@@H]([C@H]1C(=O)N[C@@H](C[C@@H]1CC(=O)NC1=O)C(=O)COc1ccc(F)cc1F)C2(C)C] |
| 45 | 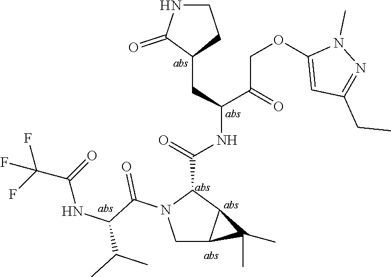 [SMILES: CCc1cc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)n(C)n1] |
| 46 | 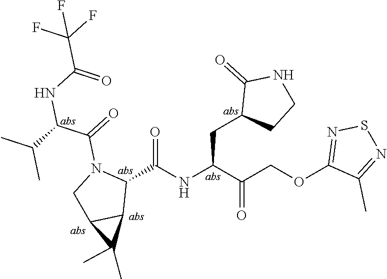 [SMILES: Cc1nsnc1OCC(=O)[C@H](C[C@@H]1CCNC1=O)NC(=O)[C@@H]1[C@@H]2[C@H](CN1C(=O)[C@@H](NC(=O)C(F)(F)F)C(C)C)C2(C)C] |
| 47 | 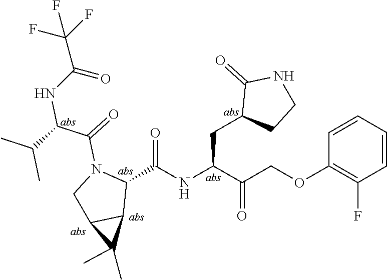 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ccccc1F)C2(C)C] |
| 48 | 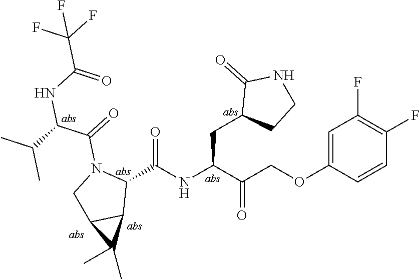 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ccc(F)c(F)c1)C2(C)C] |
| 49 | 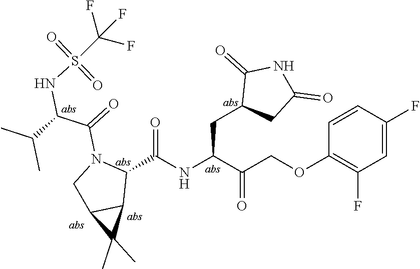 [SMILES: CC(C)[C@H](NS(=O)(=O)C(F)(F)F)C(=O)N1C[C@H]2[C@@H]([C@H]1C(=O)N[C@@H](C[C@@H]1CC(=O)NC1=O)C(=O)COc1ccc(F)cc1F)C2(C)C] |
| 50 | 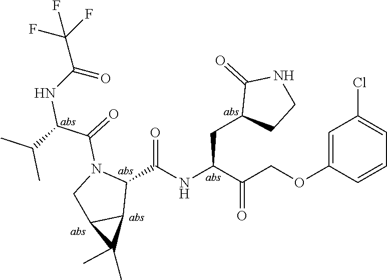 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cccc(Cl)c1)C2(C)C] |
| 51 | 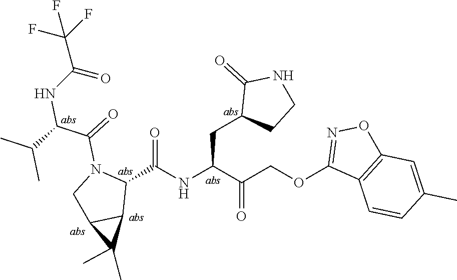 [SMILES: Cc1ccc2c(OCC(=O)[C@H](C[C@@H]3CCNC3=O)NC(=O)[C@@H]3[C@@H]4[C@H](CN3C(=O)[C@@H](NC(=O)C(F)(F)F)C(C)C)C4(C)C)noc2c1] |
| 52 | 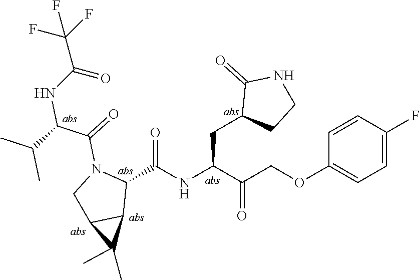 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ccc(F)cc1)C2(C)C] |
| 53 | 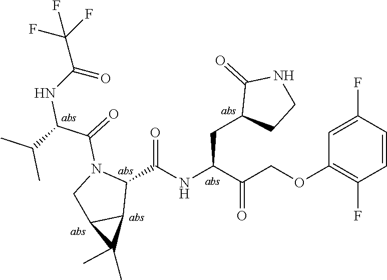 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cc(F)ccc1F)C2(C)C] |
| 54 | 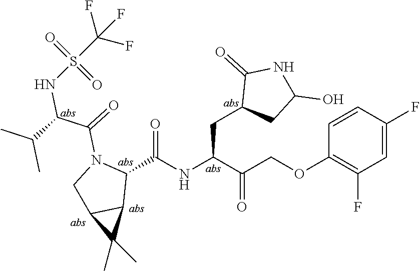 |
| 55 | 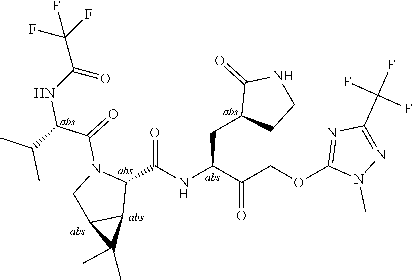 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1nc(C(F)(F)F)nn1C)C2(C)C] |
| 56 | 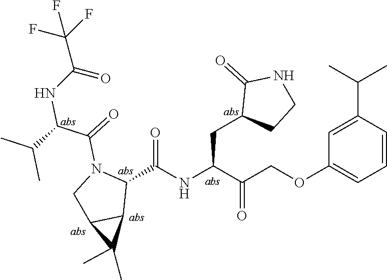 [SMILES: CC(C)c1cccc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)c1] |
| 57 | 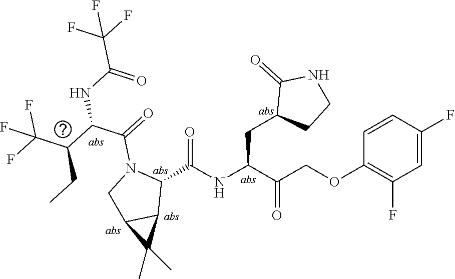 [SMILES: CC[C@H]([C@H](NC(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C)C(F)(F)F] |
| 58 | 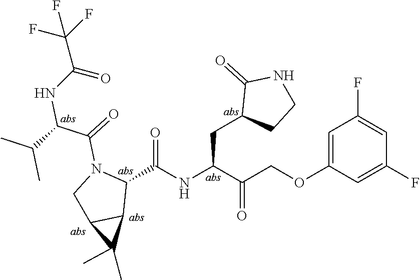 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cc(F)cc(F)c1)C2(C)C] |
| 59 | 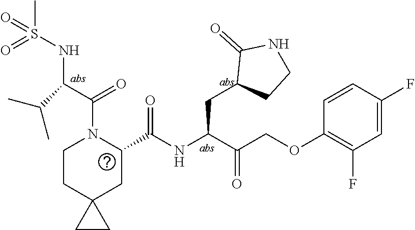 [SMILES: CC(C)[C@H](NS(C)(=O)=O)C(=O)N1CCC2(CC2)C[C@H]1C(=O)N[C@@H](C[C@@H]1CCNC1=O)C(=O)COc1ccc(F)cc1F] |
| 60 | 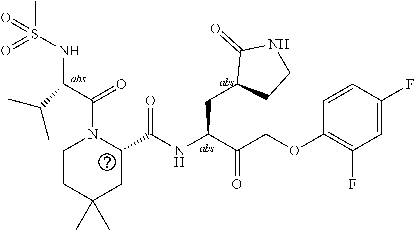 [SMILES: CC(C)[C@H](NS(C)(=O)=O)C(=O)N1CCC(C)(C)C[C@H]1C(=O)N[C@@H](C[C@@H]1CCNC1=O)C(=O)COc1ccc(F)cc1F] |
| 61 | 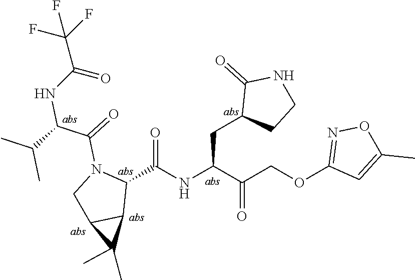 [SMILES: Cc1cc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)no1] |
| 62 | 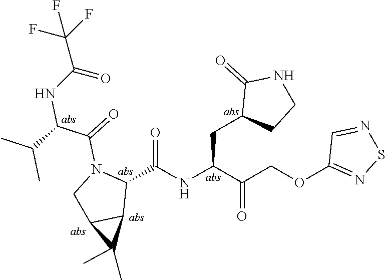 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cnsn1)C2(C)C] |
| 63 | 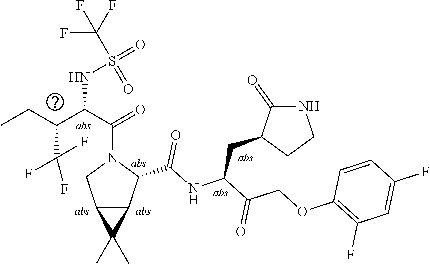 [SMILES: CC[C@H]([C@H](NS(=O)(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C)C(F)(F)F] |
| 64 | 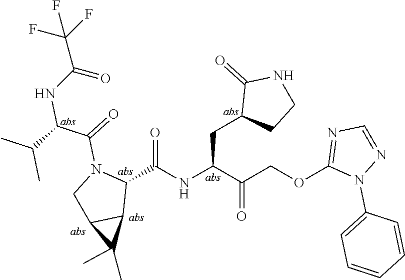 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1ncnn1-c1ccccc1)C2(C)C] |
| 65 | 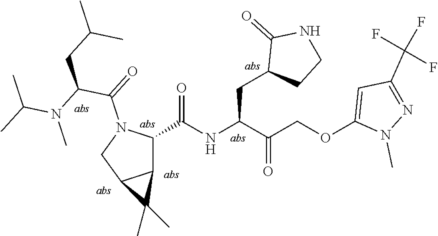 [SMILES: CC(C)C[C@@H](C(=O)N1C[C@H]2[C@@H]([C@H]1C(=O)N[C@@H](C[C@@H]1CCNC1=O)C(=O)COc1cc(C(F)(F)F)nn1C)C2(C)C)N(C)C(C)C] |
| 66 | 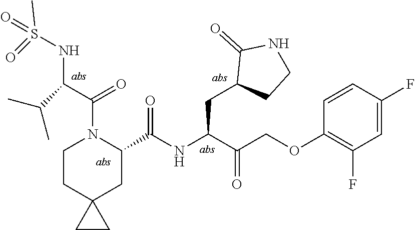 [SMILES: CC(C)[C@H](NS(C)(=O)=O)C(=O)N1CCC2(CC2)C[C@H]1C(=O)N[C@@H](C[C@@H]1CCNC1=O)C(=O)COc1ccc(F)cc1F] |
| 67 | 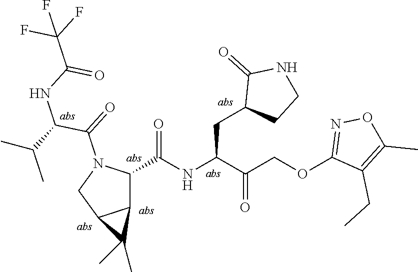 [SMILES: CCc1c(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)noc1C] |
| 68 | 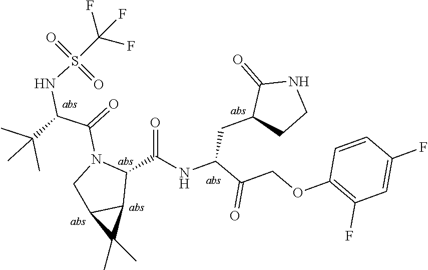 [SMILES: CC(C)(C)[C@H](NS(=O)(=O)C(F)(F)F)C(=O)N1C[C@H]2[C@@H]([C@H]1C(=O)N[C@H](C[C@@H]1CCNC1=O)C(=O)COc1ccc(F)cc1F)C2(C)C] |
| 69 | 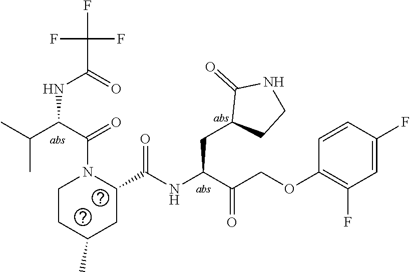 [SMILES: CC(C)[C@H](NC(=O)C(F)(F)F)C(=O)N1CC[C@@H](C)C[C@H]1C(=O)N[C@@H](C[C@@H]1CCNC1=O)C(=O)COc1ccc(F)cc1F] |
| 70 | 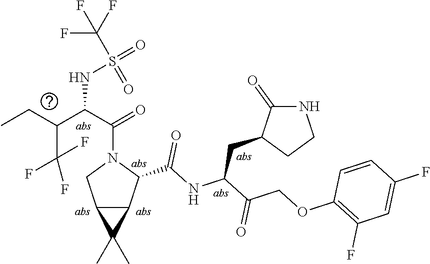 [SMILES: CCC([C@H](NS(=O)(=O)C(F)(F)F)C(=O)N1C[C@H]2[C@@H]([C@H]1C(=O)N[C@@H](C[C@@H]1CCNC1=O)C(=O)COc1ccc(F)cc1F)C2(C)C)C(F)(F)F] |
| 71 | 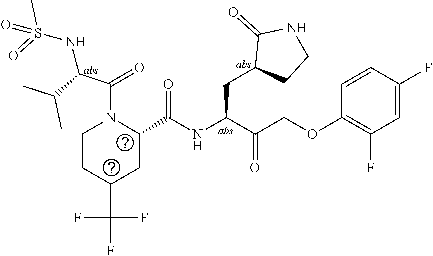 [SMILES: CC(C)[C@H](NS(C)(=O)=O)C(=O)N1CCC(C(F)(F)F)C[C@H]1C(=O)N[C@@H](C[C@@H]1CCNC1=O)C(=O)COc1ccc(F)cc1F] |
| 72 | 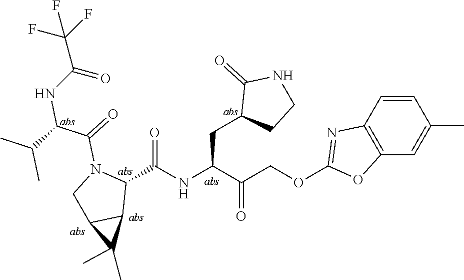 [SMILES: Cc1ccc2nc(OCC(=O)[C@H](C[C@@H]3CCNC3=O)NC(=O)[C@@H]3[C@@H]4[C@H](CN3C(=O)[C@@H](NC(=O)C(F)(F)F)C(C)C)C4(C)C)oc2c1] |
| 73 | 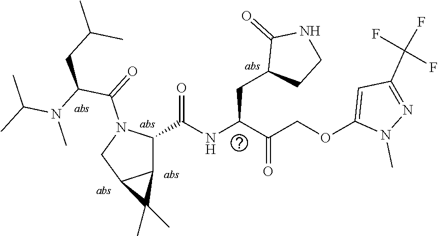 [SMILES: CC(C)C[C@@H](C(=O)N1C[C@H]2[C@@H]([C@H]1C(=O)N[C@@H](C[C@@H]1CCNC1=O)C(=O)COc1cc(C(F)(F)F)nn1C)C2(C)C)N(C)C(C)C] |
| 74 | 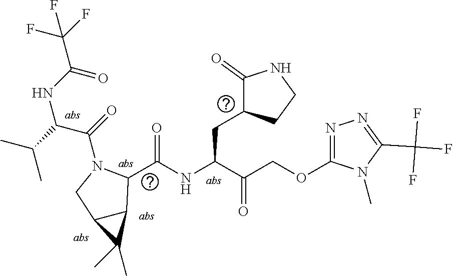 |
| 75 | 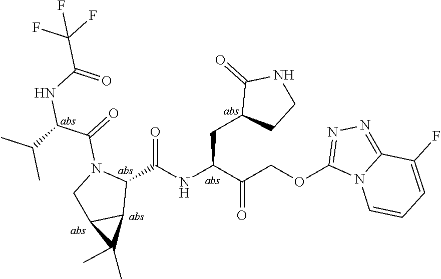 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1nnc3c(F)cccn13)C2(C)C] |
| 76 | 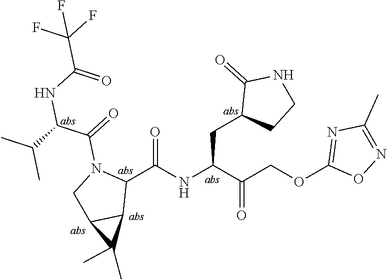 [SMILES: Cc1noc(OCC(=O)[C@H](C[C@@H]2CCNC2=O)NC(=O)C2[C@@H]3[C@H](CN2C(=O)[C@@H](NC(=O)C(F)(F)F)C(C)C)C3(C)C)n1] |
| 77 | 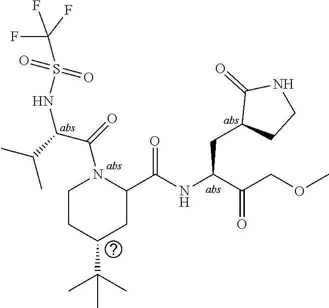 [SMILES: COCC(=O)[C@H](C[C@@H]1CCNC1=O)NC(=O)C1C[C@H](C(C)(C)C)CCN1C(=O)[C@@H](NS(=O)(=O)C(F)(F)F)C(C)C] |
| 78 | 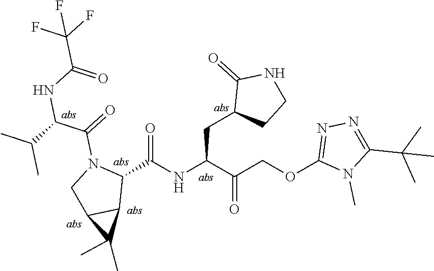 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1nnc(C(C)(C)C)n1C)C2(C)C] |
| 79 | 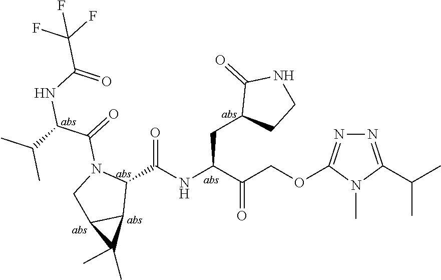 [SMILES: CC(C)c1nnc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)n1C] |
| 80 | 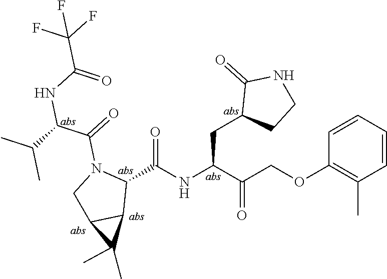 [SMILES: Cc1ccccc1OCC(=O)[C@H](C[C@@H]1CCNC1=O)NC(=O)[C@@H]1[C@@H]2[C@H](CN1C(=O)[C@@H](NC(=O)C(F)(F)F)C(C)C)C2(C)C] |
| 81 | 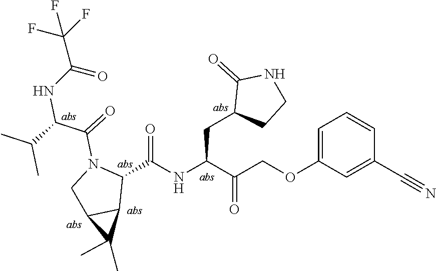 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cccc(C#N)c1)C2(C)C] |
| 82 | 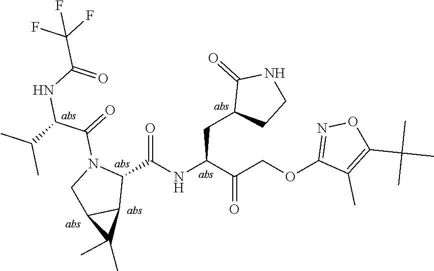 [SMILES: Cc1c(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)noc1C(C)(C)C] |
| 83 | 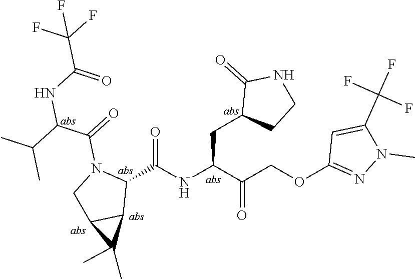 [SMILES: CC(C)C(NC(=O)C(F)(F)F)C(=O)N1C[C@H]2[C@@H]([C@H]1C(=O)N[C@@H](C[C@@H]1CCNC1=O)C(=O)COc1cc(C(F)(F)F)n(C)n1)C2(C)C] |
| 84 | 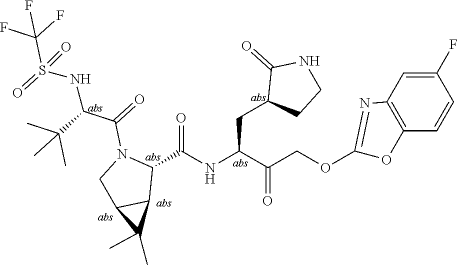 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1nc3cc(F)ccc3o1)C2(C)C] |
| 85 | 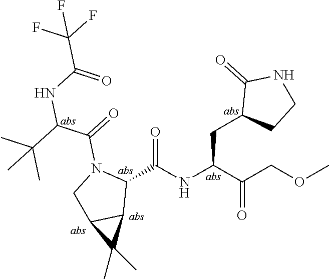 [SMILES: COCC(=O)[C@H](C[C@@H]1CCNC1=O)NC(=O)[C@@H]1[C@@H]2[C@H](CN1C(=O)C(NC(=O)C(F)(F)F)C(C)(C)C)C2(C)C] |
| 86 | 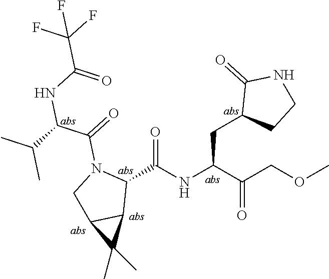 [SMILES: COCC(=O)[C@H](C[C@@H]1CCNC1=O)NC(=O)[C@@H]1[C@@H]2[C@H](CN1C(=O)[C@@H](NC(=O)C(F)(F)F)C(C)C)C2(C)C] |
| 87 | 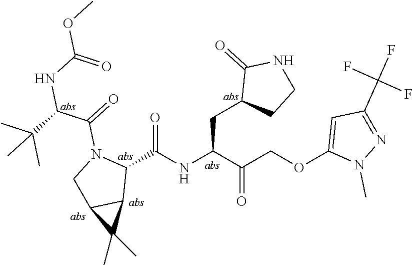 [SMILES: COC(=O)N[C@H](C(=O)N1C[C@H]2[C@@H]([C@H]1C(=O)N[C@@H](C[C@@H]1CCNC1=O)C(=O)COc1cc(C(F)(F)F)nn1C)C2(C)C)C(C)(C)C] |
| 88 | 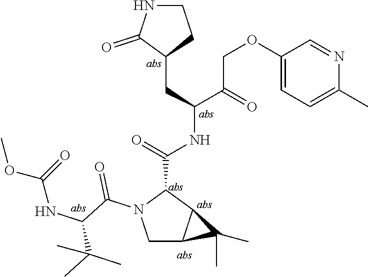 |
| 89 | |
| 90 | |
| 91 | 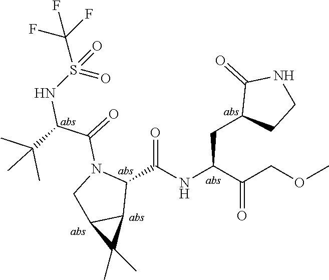 [SMILES: COCC(=O)[C@H](C[C@@H]1CCNC1=O)NC(=O)[C@@H]1[C@@H]2[C@H](CN1C(=O)[C@@H](NS(=O)(=O)C(F)(F)F)C(C)(C)C)C2(C)C] |
| 92 | 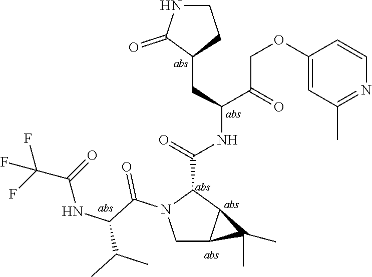 [SMILES: Cc1cc(OCC(=O)[C@H](C[C@@H]2CCNC2=O)NC(=O)[C@@H]2[C@@H]3[C@H](CN2C(=O)[C@@H](NC(=O)C(F)(F)F)C(C)C)C3(C)C)ccn1] |
| 93 | 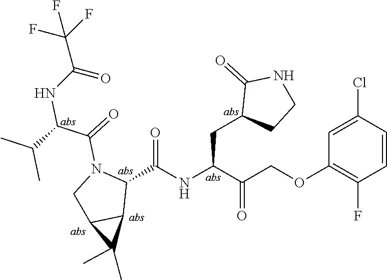 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COc1cc(Cl)ccc1F)C2(C)C] |
| 94 | 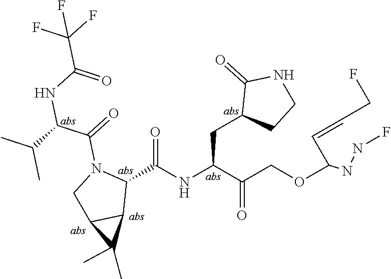 [SMILES: CC(C)[C@H](NC(=O)C(F)(F)F)C(=O)N1C[C@H]2[C@@H]([C@H]1C(=O)N[C@@H](C[C@@H]1CCNC1=O)C(=O)COC(C=CCF)NNF)C2(C)C] |
| 114 | |
| 95 | 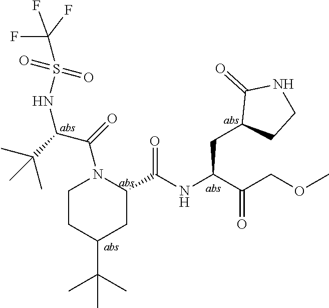 [SMILES: COCC(=O)[C@H](C[C@@H]1CCNC1=O)NC(=O)[C@@H]1CC(C(C)(C)C)CCN1C(=O)[C@@H](NS(=O)(=O)C(F)(F)F)C(C)(C)C] |
| 96 | 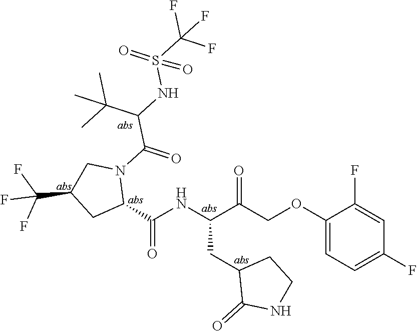 [SMILES: CC(C)(C)C(NS(=O)(=O)C(F)(F)F)C(=O)N1C[C@H](C(F)(F)F)C[C@H]1C(=O)N[C@@H](CC1CCNC1=O)C(=O)COc1ccc(F)cc1F] |
| 97 | 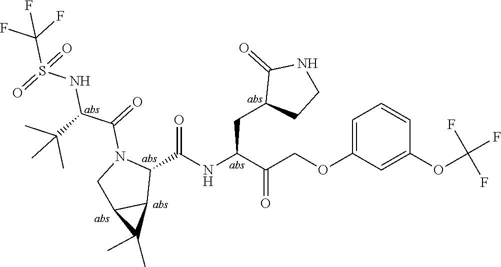 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1cccc(OC(F)(F)F)c1)C2(C)C] |
| 98 | 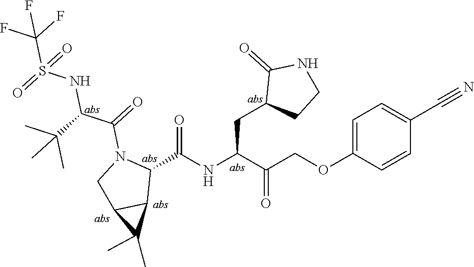 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1ccc(C#N)cc1)C2(C)C] |
| 99 | 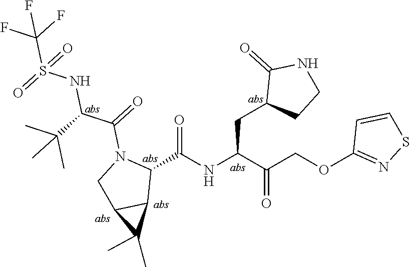 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1ccsn1)C2(C)C] |
| 100 | 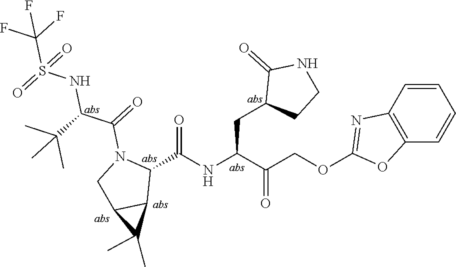 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1nc3ccccc3o1)C2(C)C] |
| 101 | 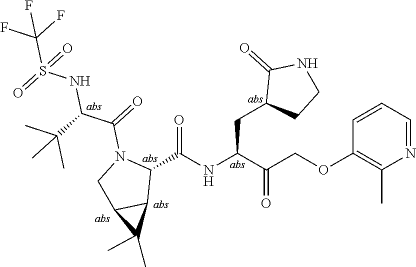 [SMILES: Cc1ncccc1OCC(=O)[C@H](C[C@@H]1CCNC1=O)NC(=O)[C@@H]1[C@@H]2[C@H](CN1C(=O)[C@@H](NS(=O)(=O)C(F)(F)F)C(C)(C)C)C2(C)C] |
| 102 | 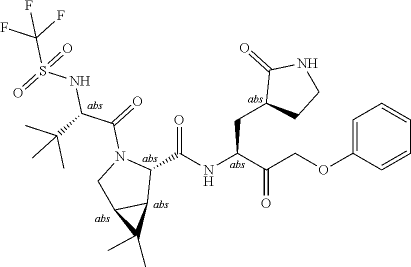 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1ccccc1)C2(C)C] |
| 103 | 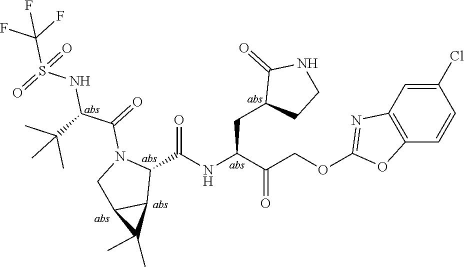 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1nc3cc(Cl)ccc3o1)C2(C)C] |
| 104 | 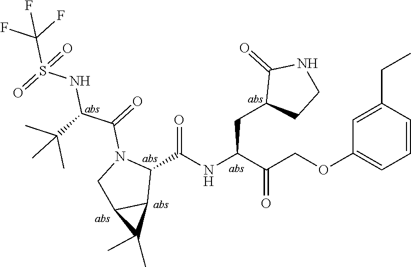 [SMILES: CCc1cccc(OCC(=O)[C@H](C[C@@H]2CCNC2=O)NC(=O)[C@@H]2[C@@H]3[C@H](CN2C(=O)[C@@H](NS(=O)(=O)C(F)(F)F)C(C)(C)C)C3(C)C)c1] |
| 105 | 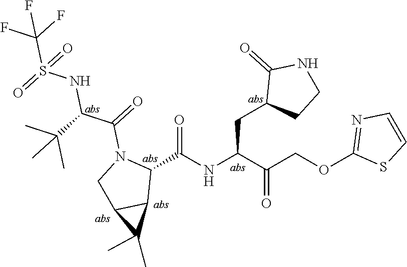 [SMILES: CC(C)(C)[C@H](NS(=O)(=O)C(F)(F)F)C(=O)N1C[C@H]2[C@@H]([C@H]1C(=O)N[C@@H](C[C@@H]1CCNC1=O)C(=O)COc1nccs1)C2(C)C] |
| 106 | 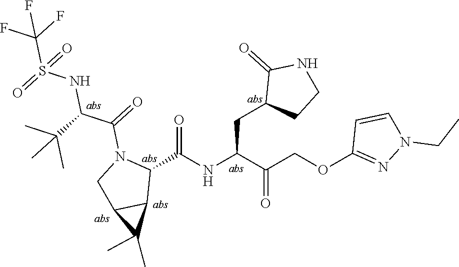 [SMILES: CCn1ccc(OCC(=O)[C@H](C[C@@H]2CCNC2=O)NC(=O)[C@@H]2[C@@H]3[C@H](CN2C(=O)[C@@H](NS(=O)(=O)C(F)(F)F)C(C)(C)C)C3(C)C)n1] |
| 107 | 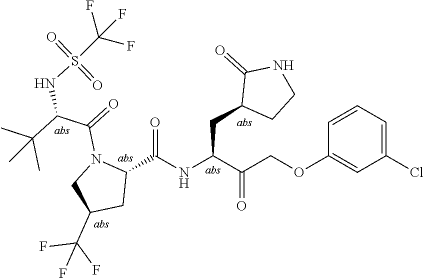 [SMILES: CC(C)(C)[C@H](NS(=O)(=O)C(F)(F)F)C(=O)N1C[C@H](C(F)(F)F)C[C@H]1C(=O)N[C@@H](C[C@@H]1CCNC1=O)C(=O)COc1cccc(Cl)c1] |
| 108 | 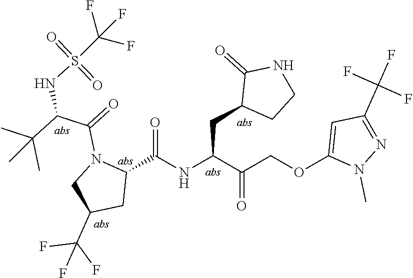 [SMILES: Cn1nc(C(F)(F)F)cc1OCC(=O)[C@H](C[C@@H]1CCNC1=O)NC(=O)[C@@H]1C[C@@H](C(F)(F)F)CN1C(=O)[C@@H](NS(=O)(=O)C(F)(F)F)C(C)(C)C] |
| 109 | |
| 110 | |
| 111 | 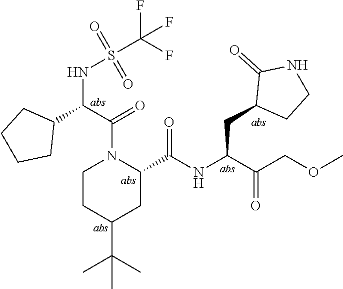 [SMILES: COCC(=O)[C@H](C[C@@H]1CCNC1=O)NC(=O)[C@@H]1CC(C(C)(C)C)CCN1C(=O)[C@@H](NS(=O)(=O)C(F)(F)F)C1CCCC1] |
| 112 | 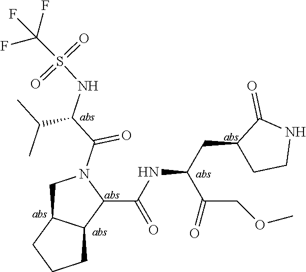 |
| 113 | 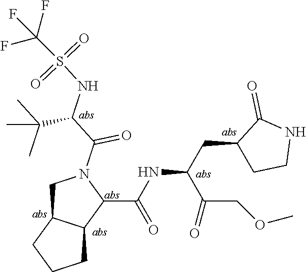 |

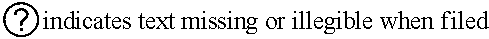

Antiviral Activity from SARS-CoV-2 Infection

The ability of compounds to prevent SARS-CoV-2 coronavirus-induced cell death or cytopathic effect can be assessed via cell viability, using an assay format that utilizes luciferase to measure intracellular ATP as an endpoint. In brief, VeroE6 cells that are enriched for hACE2 expression were batched inoculated with SARS-CoV-2 (USA_WA1/2020) at a multiplicity of infection of 0.002 in a BSL-3 lab. Virus-inoculated cells were then added to assay-ready compound plates at a density of 4,000 cells/well. Following a 3-day incubation, a time at which virus-induced cytopathic effect is 95% in the untreated, infected control conditions, cell viability was evaluated using Cell Titer-Glo (Promega), according to the manufacturer's protocol, which quantitates ATP levels. Cytotoxicity of the compounds was assessed in parallel non-infected cells. Test compounds are tested either alone or in the presence of the P-glycoprotein (P-gp) inhibitor CP-100356 at a concentration of 2 μM. The inclusion of CP-100356 is to assess if the test compounds are being effluxed out of the VeroE6 cells, which have high levels of expression of P-glycoprotein. Percent effect at each concentration of test compound was calculated based on the values for the no virus control wells and virus-containing control wells on each assay plate. The concentration required for a 50% response (EC₅₀) value was determined from these data using a 4-parameter logistic model. EC₅₀ curves were fit to a Hill slope of 3 when >3 and the top dose achieved ≥50% effect. If cytotoxicity was detected at greater than 30% effect, the corresponding concentration data was eliminated from the EC₅₀ determination.

For cytotoxicity plates, a percent effect at each concentration of test compound was calculated based on the values for the cell-only control wells and hyamine-containing control wells on each assay plate. The CC₅₀ value was calculated using a 4-parameter logistic model. A TI was then calculated by dividing the CC₅₀ value by the EC₅₀ value.

### SARS-CoV-2 Coronavirus 3C Protease FRET Assay and Analysis

The proteolytic activity of the main protease, 3CLpro, of SARS-CoV-2 was monitored using a continuous fluorescence resonance energy transfer (FRET) assay. The SARS-CoV-2 3CLpro assay measures the activity of full-length SARS-CoV-2 3CL protease to cleave a synthetic fluorogenic substrate peptide with the following sequence: Dabcyl-KTSAVLQ-SGFRKME-Edans modelled on a consensus peptide (V. Grum-Tokars et al. Evaluating the 3C-like protease activity of SARS-coronavirus: recommendations for standardized assays for drug discovery. Virus Research 133 (2008) 63-73). The fluorescence of the cleaved Edans peptide (excitation 340 nm/emission 490 nm) is measured using a fluorescence intensity protocol on a Flexstation reader (Molecular Devices). The fluorescent signal is reduced in the present of PF-835231, a potent inhibitor of SARS-CoV-2 3CLpro. The assay reaction buffer contained 20 mM Tris-HCl (pH 7.3), 100 nM NaCl, 1 mM EDTA and 25 μM peptide substrate. Enzyme reactions were initiated with the addition of 15 nM SARS-CoV-2 3CL protease and allowed to proceed for 60 minutes at 23° C. Percent inhibition or activity was calculated based on control wells containing no compound (0% inhibition/100% activity) and a control compound (100% inhibition/0% activity). IC₅₀ values were generated using a four-parameter fit model using ABASE software (IDBS). Ki values were fit to the Morrison equation with the enzyme concentration parameter fixed to 15 nM, the Km parameter fixed to 14 μM and the substrate concentration parameter fixed to 25 μM using ABASE software (IDBS).

Proteolytic activity of SARS-CoV-2 Coronavirus 3CL protease is measured using a continuous fluorescence resonance energy transfer assay. The SARS-CoV-2 3CL^{pro} FRET assay measures the protease catalyzed cleavage of TAMRA-SITSAVLQSGFRKMK-(DABCYL)-OH to TAMRA-SITSAVLQ and SGFRKMK(DABCYL)-OH. The fluorescence of the cleaved TAMRA (ex. 558 nm/em. 581 nm) peptide was measured using a TECAN SAFIRE fluorescence plate reader over the course of 10 min. Typical reaction solutions contained 20 mM HEPES (pH 7.0), 1 mM EDTA, 4.0 μM FRET substrate, 4% DMSO and 0.005% Tween-20. Assays were initiated with the addition of 25 nM SARS 3CL^{pro} (nucleotide sequence 9985-10902 of the Urbani strain of SARS coronavirus complete genome sequence (NCBI accession number AY278741)). Percent inhibition was determined in duplicate at 0.001 mM level of inhibitor. Data was analyzed with the non-linear regression analysis program Kalidagraph using the equation:

$$FU = offset + ( limit ) ( 1 - {e}^{- ( kobs )} t )$$

where offset equals the fluorescence signal of the un-cleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitor, and where the calculated limit is less than 20% of the theoretical maximum limit, the calculated kobs represents the rate of inactivation of coronavirus 3C protease. The slope (kobs/l) of a plot of kobs vs. [l] is a measure of the avidity of the inhibitor for an enzyme. For very fast irreversible inhibitors, kobs/l is calculated from observations at only one or two [l] rather than as a slope.

TABLE 2
Biological activity and IUPAC name for Examples 1-78.

| | | | Geometric | Count | |
|---|---|---|---|---|---|
| | Geometric | Count | Mean | Used | |
| | Mean | Used | EC₅₀ | EC₅₀ | |
| Example | Kᵢ (nM) | Kᵢ (nM) | (nM) | (nM) | IUPAC Name |
| 1 | 2.02 | 5 | 26.9 | 7 | (1R,2S,5S)-N-{(2S)-4-(2,4-difluorophenoxy)-3-oxo-1- |
| | | | | | [(3S)-2-oxopyrrolidin-3-yl]butan-2-yl}-6,6-dimethyl-3- |
| | | | | | [N-(trifluoroacetyl)-L-valyl]-3-azabicyclo[3.1.0]hexane- |
| | | | | | 2-carboxamide |
| 2 | 172 | 2 | 74.4 | 2 | (1R,2S,5S)-N-{(2R)-4-(2,4-difluorophenoxy)-3-oxo-1- |
| | | | | | [(3S)-2-oxopyrrolidin-3-yl]butan-2-yl}-6,6-dimethyl-3- |
| | | | | | [N-(trifluoroacetyl)-L-valyl]-3-azabicyclo[3.1.0]hexane- |
| | | | | | 2-carboxamide |
| 3 | 3.50 | 4 | 207 | 8 | (1R,2S,5S)-N-{(2S)-4-(2,4-difluorophenoxy)-3-oxo-1- |
| | | | | | [(3S)-2-oxopyrrolidin-3-yl]butan-2-yl}-6,6-dimethyl-3- |
| | | | | | {3-methyl-N-[(trifluoromethyl)sulfonyl]-L-valyl}-3- |
| | | | | | azabicyclo[3.1.0]hexane-2-carboxamide |
| 4 | 6.34 | 2 | 395 | 6 | (1R,2S,5S)-N-{(2S)-4-(2,4-difluorophenoxy)-3-oxo-1- |
| | | | | | [(3S)-2-oxopyrrolidin-3-yl]butan-2-yl}-6,6-dimethyl-3- |
| | | | | | {N-[(trifluoromethyl)sulfonyl]-L-valyl}-3- |
| | | | | | azabicyclo[3.1.0]hexane-2-carboxamide |
| 5 | 10.6 | 2 | 205 | 4 | (1R,2S,5S)-6,6-dimethyl-N-{(2S)-3-oxo-1-[(3S)-2- |
| | | | | | oxopyrrolidin-3-yl]-4-[3-(trifluoromethyl)phenoxy]butan- |
| | | | | | 2-yl}-3-{N-[(trifluoromethyl)sulfonyl]-L-valyl}-3- |
| | | | | | azabicyclo[3.1.0]hexane-2-carboxamide |
| 6 | 19.8 | 2 | 735 | 8 | (1R,2S,5S)-6,6-dimethyl-N-{(2S)-4-{[1-methyl-3- |
| | | | | | (trifluoromethyl)-1H-pyrazol-5-yl]oxy}-3-oxo-1- |
| | | | | | [(3S)-2-oxopyrrolidin-3-yl]butan-2-yl}-3-{3-methyl- |
| | | | | | N-[(trifluoromethyl)sulfonyl]-L-valyl}-3- |
| | | | | | azabicyclo[3.1.0]hexane-2-carboxamide |
| 7 | 2.88 | 2 | 1260 | 6 | (1R,2S,5S)-6,6-dimethyl-3-[3-methyl-N-(methylsulfonyl)- |
| | | | | | L-valyl]-N-{(2S)-4-{[1-methyl-3-(trifluoromethyl)-1H- |
| | | | | | pyrazol-5-yl]oxy}-3-oxo-1-[(3S)-2-oxopyrrolidin-3- |
| | | | | | yl]butan-2-yl}-3-azabicyclo[3.1.0]hexane-2-carboxamide |
| 8 | 2.87 | 3 | 49.1 | 4 | (1R,2S,5S)-6,6-dimethyl-3-[3-methyl-N-(trifluoroacetyl)- |
| | | | | | L-valyl]-N-{(2S)-4-{[1-methyl-3-(trifluoromethyl)-1H- |
| | | | | | pyrazol-5-yl]oxy}-3-oxo-1-[(3S)-2-oxopyrrolidin-3- |
| | | | | | yl]butan-2-yl}-3-azabicyclo[3.1.0]hexane-2-carboxamide |
| 9 | 42.8 | 2 | 265 | 5 | (1R,2S,5S)-6,6-dimethyl-N-{(2S)-4-{[1-methyl-3- |
| | | | | | (trifluoromethyl)-1H-pyrazol-5-yl]oxy}-3-oxo-1-[(3S)-2- |
| | | | | | oxopyrrolidin-3-yl]butan-2-yl}-3-[N-(trifluoroacetyl)-L- |
| | | | | | valyl]-3-azabicyclo[3.1.0]hexane-2-carboxamide, |
| | | | | | trifluoroacetate salt |
| 10 | 8.34 | 2 | 167 | 4 | (1R,2S,5S)-6,6-dimethyl-3-{3-methyl-N- |
| | | | | | [(trifluoromethyl)sulfonyl]-L-valyl}-N-{(2S)-3- |
| | | | | | oxo-1-[(3S)-2-oxopyrrolidin-3-yl]-4-[3- |
| | | | | | (trifluoromethyl)phenoxy]butan-2-yl}-3- |
| | | | | | azabicyclo[3.1.0]hexane-2-carboxamide |
| 11 | 13.5 | 2 | 2030 | 4 | (1R,2S,5S)-6,6-dimethyl-N-{(2S)-4-{[1-methyl- |
| | | | | | 3-(trifluoromethyl)-1H-pyrazol-5-yl]oxy}-3-oxo- |
| | | | | | 1-[(3S)-2-oxopyrrolidin-3-yl]butan-2-yl}-3-{N- |
| | | | | | [(trifluoromethyl)sulfonyl]-L-valyl}-3- |
| | | | | | azabicyclo[3.1.0]hexane-2-carboxamide |
| 12 | 6.38 | 2 | 51.2 | 8 | (1R,2S,5S)-6,6-dimethyl-3-[3-methyl-N-(trifluoroacetyl)- |
| | | | | | L-valyl]-N-{(2S)-3-oxo-1-[(3S)-2-oxopyrrolidin-3-yl]-4- |
| | | | | | [3-(trifluoromethyl)phenoxy]butan-2-yl}-3- |
| | | | | | azabicyclo[3.1.0]hexane-2-carboxamide |
| 13 | 1.78 | 2 | 98.6 | 6 | (1R,2S,5S)-N-{(2S)-4-(2,4-difluorophenoxy)-3-oxo-1- |
| | | | | | [(3S)-2-oxopyrrolidin-3-yl]butan-2-yl}-6,6-dimethyl-3- |
| | | | | | [3-methyl-N-(methylsulfonyl)-L-valyl]-3- |
| | | | | | azabicyclo[3.1.0]hexane-2-carboxamide |

| | Geometric | | | Count | |
|---|---|---|---|---|---|
| | Mean | Count | Geometric | Used | |
| | Ki (nM) | Used | Mean | EC50 | |
| Example | IC50 | Ki (nM) | EC50 (nM) | (nM) | IUPAC Name |
| 14 | 3.48 | 3 | 48.7 | 2 | (1R,2S,5S)-N-{(2S)-4-[(4- |
| | | | | | methoxyphenyl)methoxy]-3-oxo-1-[(3S)-2- |
| | | | | | oxopyrrolidin-3-yl]butan-2-yl}-6,6- |
| | | | | | dimethyl-3-[3-methyl-N-(trifluoroacetyl)- |
| | | | | | L-valyl]-3-azabicyclo[3.1.0]hexane- |
| | | | | | 2-carboxamide |
| 15 | 5.58 | 2 | 272 | 6 | (2R*,4S*)-4-tert-butyl-N-{(2S)-4-methoxy- |
| | | | | | 3-oxo-1-[(3S)-2-oxopyrrolidin-3-yl]butan- |
| | | | | | 2-yl}-1-[N-(trifluoromethanesulfonyl)-L- |
| | | | | | valyl]piperidine-2-carboxamide, DIAST-1 |
| 16 | 0.210 | 2 | 11.6 | 4 | methyl {(2S)-1-[(1R,2S,5S)-2-({(2S)-4-(3- |
| | | | | | chlorophenoxy)-3-oxo-1-[(3S)-2- |
| | | | | | oxopyrrolidin-3-yl]butan-2-yl}carbamoyl)- |
| | | | | | 6,6-dimethyl-3-azabicyclo[3.1.0]hexan-3- |
| | | | | | yl]-3,3-dimethyl-1-oxobutan-2-yl}carbamate |
| 17 | <0.443 | 3 | 13.3 | 6 | methyl {(2S)-1-[(1R,2S,5S)-6,6-dimethyl-2- |
| | | | | | ({(2S)-3-oxo-1-[(3S)-2-oxopyrrolidin-3- |
| | | | | | yl]-4-[3-(trifluoromethyl)phenoxy]butan-2- |
| | | | | | yl}carbamoyl)-3-azabicyclo[3.1.0]hexan-3- |
| | | | | | yl]-3,3-dimethyl-1-oxobutan-2-yl}carbamate |
| 18 | N.D¹ | | 114 | 2 | (1R,2S,5S)-6,6-dimethyl-N-{(2S)-4-[(6- |
| | | | | | methylpyridin-3-yl)oxy]-3-oxo-1-[(3S)-2- |
| | | | | | oxopyrrolidin-3-yl]butan-2-yl}-3-[N- |
| | | | | | (trifluoroacetyl)-L-valyl]-3- |
| | | | | | azabicyclo[3.1.0]hexane-2-carboxamide |
| 19 | N.D. | | 37.2 | 4 | (1R,2S,5S)-6,6-dimethyl-N-{(2S)-4-[(6- |
| | | | | | methylpyridin-3-yl)oxy]-3-oxo-1-[(3S)-2- |
| | | | | | oxopyrrolidin-3-yl]butan-2-yl}-3-[3-methyl- |
| | | | | | N-(trifluoroacetyl)-L-valyl]-3- |
| | | | | | azabicyclo[3.1.0]hexane-2-carboxamide |
| 20 | N.D. | | 14.7 | 4 | (1R,2S,5S)-6,6-dimethyl-3-[3-methyl-N- |
| | | | | | (trifluoroacetyl)-L-valyl]-N-{(2S)-3-oxo-1- |
| | | | | | [(3S)-2-oxopyrrolidin-3-yl]-4-phenoxybutan- |
| | | | | | 2-yl}-3-azabicyclo[3.1.0]hexane- |
| | | | | | 2-carboxamide |
| 21 | 2.20 | 2 | 18.8 | 2 | methyl {(2S)-1-[(1R,2S,5S)-2-({(2S)-4-(4- |
| | | | | | fluorophenoxy)-3-oxo-1-[(3S)-2- |
| | | | | | oxopyrrolidin-3-yl]butan-2-yl}carbamoyl)- |
| | | | | | 6,6-dimethyl-3-azabicyclo[3.1.0]hexan-3- |
| | | | | | yl]-3,3-dimethyl-1-oxobutan-2-yl}carbamate |
| 22 | 4.78 | 2 | 52 | 2 | methyl {(2S)-1-[(1R,2S,5S)-2-({(2S)-4-(2- |
| | | | | | fluorophenoxy)-3-oxo-1-[(3S)-2- |
| | | | | | oxopyrrolidin-3-yl]butan-2-yl}carbamoyl)- |
| | | | | | 6,6-dimethyl-3-azabicyclo[3.1.0]hexan-3- |
| | | | | | yl]-3,3-dimethyl-1-oxobutan-2-yl}carbamate |
| 23 | 1.78 | 1 | 32 | 4 | (1R,2S,5S)-6,6-dimethyl-N-{(2S)-3-oxo-1- |
| | | | | | [(3S)-2-oxopyrrolidin-3-yl]-4-phenoxybutan- |
| | | | | | 2-yl}-3-[N-(trifluoroacetyl)-L-valyl]-3- |
| | | | | | azabicyclo[3.1.0]hexane-2-carboxamide |
| 24 | 1.41 | 2 | 17 | 4 | methyl {(2S)-1-[(1R,2S,5S)-2-({(2S)-4-(2,4- |
| | | | | | difluorophenoxy)-3-oxo-1-[(3S)-2- |
| | | | | | oxopyrrolidin-3-yl]butan-2-yl}carbamoyl)- |
| | | | | | 6,6-dimethyl-3-azabicyclo[3.1.0]hexan-3- |
| | | | | | yl]-3,3-dimethyl-1-oxobutan-2-yl}carbamate |
| 25 | 1.76 | 1 | 38.2 | 4 | methyl {(2S)-1-[(1R,2S,5S)-2-({(2S)-4-(3- |
| | | | | | chlorophenoxy)-3-oxo-1-[(3S)-2- |
| | | | | | oxopyrrolidin-3-yl]butan-2-yl}carbamoyl)- |
| | | | | | 6,6-dimethyl-3-azabicyclo[3.1.0]hexan-3- |
| | | | | | yl]-3-methyl-1-oxobutan-2-yl}carbamate |
| 26 | N.D. | | 171 | 2 | (1R,2S,5S)-N-{(2S)-4-[(3-ethyl-1-methyl- |
| | | | | | 1H-pyrazol-5-yl)oxy]-3-oxo-1-[(3S)-2- |
| | | | | | oxopyrrolidin-3-yl]butan-2-yl}-6,6-dimethyl- |
| | | | | | 3-[3-methyl-N-(trifluoroacetyl)-L-valyl]-3- |
| | | | | | azabicyclo[3.1.0]hexane-2-carboxamide |
| 27 | 8.84 | 1 | 422 | 2 | (1R,2S,5S)-N-{(2S)-4-[(1-ethyl-3-methyl- |
| | | | | | 1H-pyrazol-5-yl)oxy]-3-oxo-1-[(3S)-2- |
| | | | | | oxopyrrolidin-3-yl]butan-2-yl}-6,6-dimethyl- |
| | | | | | 3-[3-methyl-N-(trifluoroacetyl)-L-valyl]-3- |
| | | | | | azabicyclo[3.1.0]hexane-2-carboxamide |
| 28 | 2.15 | 2 | 16.8 | 2 | (1R,2S,5S)-N-{(2S)-4-(3-chlorophenoxy)-3- |
| | | | | | oxo-1-[(3S)-2-oxopyrrolidin-3-yl]butan-2- |
| | | | | | yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |
| 29 | 4.69 | 2 | 57 | 2 | (1R,2S,5S)-N-{(2S)-4-(4-fluorophenoxy)-3- |
| | | | | | oxo-1-[(3S)-2-oxopyrrolidin-3-yl]butan-2- |
| | | | | | yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |
| 30 | 2.15 | 1 | 27.2 | 4 | methyl{(2S)-1-[(1R,2S,5S)-6,6-dimethyl-2- |
| | | | | | ({(2S)-3-oxo-1-[(3S)-2-oxopyrrolidin-3-yl]- |
| | | | | | 4-phenoxybutan-2-yl}carbamoyl)-3- |
| | | | | | azabicyclo[3.1.0]hexan-3-yl]-3,3-dimethyl- |
| | | | | | 1-oxobutan-2-yl}carbamate |
| 31 | N.D. | | 167 | 2 | (1R,2S,5S)-N-{(2S)-4-[(6-fluoropyridin-3- |
| | | | | | yl)oxy]-3-oxo-1-[(3S)-2-oxopyrrolidin-3- |
| | | | | | yl]butan-2-yl}-6,6-dimethyl-3-[N- |
| | | | | | (trifluoroacetyl)-L-valyl]-3- |
| | | | | | azabicyclo[3.1.0]hexane-2-carboxamide |
| 32 | 2.42 | 2 | 98.9 | 4 | (1R,2S,5S)-N-{(2S)-4-(2,4-difluorophenoxy)- |
| | | | | | 3-oxo-1-[(3S)-2-oxopyrrolidin-3-yl]butan- |
| | | | | | 2-yl}-6,6-dimethyl-3-[3′,3′,3′-trifluoro-N- |
| | | | | | (trifluoroacetyl)-L-isoleucyl]-3- |
| | | | | | azabicyclo[3.1.0]hexane-2-carboxamide |
| 33 | 3.04 | 1 | 1270 | 2 | methyl {(2S)-1-[(1R,2S,5S)-2-({(2S)-4-[(1- |
| | | | | | ethyl-3-methyl-1H-pyrazol-5-yl)oxy]-3-oxo- |
| | | | | | 1-[(3S)-2-oxopyrrolidin-3-yl]butan-2- |
| | | | | | yl}carbamoyl)-6,6-dimethyl-3- |
| | | | | | azabicyclo[3.1.0]hexan-3-yl]-3,3-dimethyl- |
| | | | | | 1-oxobutan-2-yl}carbamate |
| 34 | 6.95 | 2 | 86 | 2 | (1R,2S,5S)-N-{(2S)-4-(2,5-difluorophenoxy)- |
| | | | | | 3-oxo-1-[(3S)-2-oxopyrrolidin-3-yl]butan-2- |
| | | | | | yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |
| 35 | 2.76 | 2 | 93.8 | 10 | (1R,2S,5S)-N-{(2RS)-4-(2,4- |
| | | | | | difluorophenoxy)-3-oxo-1-[(3S)-2- |
| | | | | | oxopyrrolidin-3-yl]butan-2-yl}-6,6-dimethyl- |
| | | | | | 3-[N-(trifluoroacetyl)-L-valyl]-3- |
| | | | | | azabicyclo[3.1.0]hexane-2-carboxamide |
| 36 | N.D. | | 256 | 2 | methyl {(2S)-1-[(1R,2S,5S)-6,6-dimethyl-2- |
| | | | | | ({(2S)-4-{[1-methyl-3-(trifluoromethyl)-1H- |
| | | | | | pyrazol-5-yl]oxy}-3-oxo-1-[(3S)-2- |
| | | | | | oxopyrrolidin-3-yl]butan-2-yl}carbamoyl)-3- |
| | | | | | azabicyclo[3.1.0]hexan-3-yl]-3-methyl-1- |
| | | | | | oxobutan-2-yl}carbamate |
| 37 | N.D. | | 900 | 2 | methyl {(2S)-1-[(1R,2S,5S)-2-({(2S)-4-[(3- |
| | | | | | ethyl-1-methyl-1H-pyrazol-5-yl)oxy]-3-oxo- |
| | | | | | 1-[(3S)-2-oxopyrrolidin-3-yl]butan-2- |
| | | | | | yl}carbamoyl)-6,6-dimethyl-3- |
| | | | | | azabicyclo[3.1.0]hexan-3-yl]-3,3-dimethyl-1- |
| | | | | | oxobutan-2-yl}carbamate |
| 38 | 4.12 | 2 | 1390 | 2 | (1R,2S,5S)-N-{(2S)-4-(2,4-difluorophenoxy)- |
| | | | | | 1-[(3R)-5-hydroxy-2-oxopyrrolidin-3-yl]-3- |
| | | | | | oxobutan-2-yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |
| 39 | 3.70 | 1 | 306 | 2 | (1R,2S,5S)-N-{(2S)-4-(2,4-difluorophenoxy)- |
| | | | | | 1-[(3R)-5-hydroxy-2-oxopyrrolidin-3-yl]-3- |
| | | | | | oxobutan-2-yl}-6,6-dimethyl-3-[N- |
| | | | | | (trifluoroacetyl)-L-valyl]-3- |
| | | | | | azabicyclo[3.1.0]hexane-2-carboxamide |
| 40 | 4.22 | 1 | 112 | 2 | (1R,2S,5S)-N-{(2S)-4-(3-chlorophenoxy)- |
| | | | | | 3-oxo-1-[(3S)-2-oxopyrrolidin-3-yl]butan- |
| | | | | | 2-yl}-6,6-dimethyl-3-[N- |
| | | | | | (trifluoromethanesulfonyl)-L-valyl]-3- |
| | | | | | azabicyclo[3.1.0]hexane-2-carboxamide |
| 41 | 5.45 | 3 | 224 | 2 | (2R*,4S*)-N-{(2S)-4-(2,4-difluorophenoxy)- |
| | | | | | 3-oxo-1-[(3S)-2-oxopyrrolidin-3-yl]butan-2- |
| | | | | | yl}-1-[N-(methanesulfonyl)-L-valyl]-4- |
| | | | | | (trifluoromethyl)piperidine-2-carboxamide, |
| | | | | | DIAST-2 |
| 42 | 7.05 | 1 | 301 | 2 | (1R,2S,5S)-N-{(2S)-4-(2,4-difluorophenoxy)- |
| | | | | | 1-[(3R)-2,5-dioxopyrrolidin-3-yl]-3- |
| | | | | | oxobutan-2-yl}-6,6-dimethyl-3-[N- |
| | | | | | (trifluoroacetyl)-L-valyl]-3- |
| | | | | | azabicyclo[3.1.0]hexane-2-carboxamide |
| 43 | N.D. | | 642 | 2 | (1R,2S,5S)-N-{(2S)-4-[(1-ethyl-3-methyl- |
| | | | | | 1H-pyrazol-5-yl)oxy]-3-oxo-1-[(3S)-2- |
| | | | | | oxopyrrolidin-3-yl]butan-2-yl}-6,6-dimethyl- |
| | | | | | 3-[N-(trifluoroacetyl)-L-valyl]-3- |
| | | | | | azabicyclo[3.1.0]hexane-2-carboxamide |
| 44 | 8.95 | 2 | 408 | 2 | (1R,2S,5S)-N-{(2S)-4-(2,4-difluorophenoxy)- |
| | | | | | 1-[(3R)-2,5-dioxopyrrolidin-3-yl]-3- |
| | | | | | oxobutan-2-yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |
| 45 | N.D. | | 384 | 2 | (1R,2S,5S)-N-{(2S)-4-[(3-ethyl-1-methyl-1H- |
| | | | | | pyrazol-5-yl)oxy]-3-oxo-1-[(3S)-2- |
| | | | | | oxopyrrolidin-3-yl]butan-2-yl}-6,6-dimethyl- |
| | | | | | 3-[N-(trifluoroacetyl)-L-valyl]-3- |
| | | | | | azabicyclo[3.1.0]hexane-2-carboxamide |
| 46 | 8.21 | 2 | N.D. | | (1R,2S,5S)-6,6-dimethyl-N-{(2S)-4-[(4- |
| | | | | | methyl-1,2,5-thiadiazol-3-yl)oxy]-3-oxo-1- |
| | | | | | [(3S)-2-oxopyrrolidin-3-yl]butan-2-yl}-3-[N- |
| | | | | | (trifluoroacetyl)-L-valyl]-3- |
| | | | | | azabicyclo[3.1.0]hexane-2-carboxamide |
| 47 | 8.58 | 2 | 59.7 | 8 | (1R,2S,5S)-N-{(2S)-4-(2-fluorophenoxy)-3- |
| | | | | | oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 48 | 7.87 | 2 | 44.3 | 8 | (1R,2S,5S)-N-{(2S)-4-(3,4-difluorophenoxy)- |
| | | | | | 3-oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 49 | 9.93 | 1 | 2010 | 2 | (1R,2S,5S)-N-{(2S)-4-(2,4-difluorophenoxy)- |
| | | | | | 1-[(3R)-2,5-dioxopyrrolidin-3-yl]-3- |
| | | | | | oxobutan-2-yl}-6,6-dimethyl-3-[N- |
| | | | | | (trifluoromethanesulfonyl)-L-valyl]-3- |
| | | | | | azabicyclo[3.1.0]hexane-2-carboxamide |
| 50 | 10.7 | 2 | 76.2 | 8 | (1R,2S,5S)-N-{(2S)-4-(3-chlorophenoxy)-3- |
| | | | | | oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 51 | 10.5 | 2 | 94.2 | 8 | (1R,2S,5S)-6,6-dimethyl-N-{(2S)-4-[(6- |
| | | | | | methyl-1,2-benzoxazol-3-yl)oxy]-3-oxo-1- |
| | | | | | [(3S)-2-oxopyrrolidin-3-yl]butan-2-yl}-3-[N- |
| | | | | | (trifluoroacetyl)-L-valyl]-3- |
| | | | | | azabicyclo[3.1.0]hexane-2-carboxamide |
| 52 | 10.5 | 2 | 73.3 | 8 | (1R,2S,5S)-N-{(2S)-4-(4-fluorophenoxy)-3- |
| | | | | | oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 53 | 12 | 2 | 72.8 | 8 | (1R,2S,5S)-N-{(2S)-4-(2,5-difluorophenoxy)- |
| | | | | | 3-oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 54 | 11.3 | 1 | 2250 | 2 | (1R,2S,5S)-N-{(2S)-4-(2,4-difluorophenoxy)- |
| | | | | | 1-[(3R)-5-hydroxy-2-oxopyrrolidin-3-yl]-3- |
| | | | | | oxobutan-2-yl}-6,6-dimethyl-3-[N- |
| | | | | | (trifluoromethanesulfonyl)-L-valyl]-3- |
| | | | | | azabicyclo[3.1.0]hexane-2-carboxamide |
| 55 | 11.6 | 2 | 642 | 8 | (1R,2S,5S)-6,6-dimethyl-N-{(2S)-4-{[1- |
| | | | | | methyl-3-(trifluoromethyl)-1H-1,2,4-triazol- |
| | | | | | 5-yl]oxy}-3-oxo-1-[(3S)-2-oxopyrrolidin-3- |
| | | | | | yl]butan-2-yl}-3-[N-(trifluoroacetyl)-L- |
| | | | | | valyl]-3-azabicyclo[3.1.0]hexane- |
| | | | | | 2-carboxamide |
| 56 | 14.4 | 1 | 119 | 4 | (1R,2S,5S)-6,6-dimethyl-N-{(2S)-3-oxo-1- |
| | | | | | [(3S)-2-oxopyrrolidin-3-yl]-4-[3-(propan-2- |
| | | | | | yl)phenoxy]butan-2-yl}-3-[N- |
| | | | | | (trifluoroacetyl)-L-valyl]-3- |
| | | | | | azabicyclo[3.1.0]hexane-2-carboxamide |
| 57 | 17.3 | 2 | 275 | 4 | (1R,2S,5S)-N-{(2S)-4-(2,4-difluorophenoxy)- |
| | | | | | 3-oxo-1-[(3S)-2-oxopyrrolidin-3-yl]butan-2- |
| | | | | | yl}-6,6-dimethyl-3-[3′,3′,3′-trifluoro-N- |
| | | | | | (trifluoroacetyl)-L-isoleucyl]-3- |
| | | | | | azabicyclo[3.1.0]hexane-2-carboxamide |
| 58 | 17.1 | 2 | 88.7 | 8 | (1R,2S,5S)-N-{(2S)-4-(3,5-difluorophenoxy)- |
| | | | | | 3-oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 59 | 9.27 | 2 | 299 | 2 | (5R*)-N-{(2S)-4-(2,4-difluorophenoxy)-3- |
| | | | | | oxo-1-[(3S)-2-oxopyrrolidin-3-yl]butan-2- |
| | | | | | yl}-6-[N-(methanesulfonyl)-L-valyl]-6- |
| | | | | | azaspiro[2.5]octane-5-carboxamide, DIAST-1 |
| 60 | 21.8 | 2 | 456 | 6 | (2R*)-N-{(2S)-4-(2,4-difluorophenoxy)-3- |
| | | | | | oxo-1-[(3S)-2-oxopyrrolidin-3-yl]butan-2- |
| | | | | | yl}-1-[N-(methanesulfonyl)-L-valyl]-4,4- |
| | | | | | dimethylpiperidine-2-carboxamide |
| 61 | 31.7 | 2 | 1800 | 8 | (1R,2S,5S)-6,6-dimethyl-N-{(2S)-4-[(5- |
| | | | | | methyl-1,2-oxazol-3-yl)oxy]-3-oxo-1-[(3S)- |
| | | | | | 2-oxopyrrolidin-3-yl]butan-2-yl}-3-[N- |
| | | | | | (trifluoroacetyl)-L-valyl]-3- |
| | | | | | azabicyclo[3.1.0]hexane-2-carboxamide |
| 62 | 55.7 | 2 | 1870 | 8 | (1R,2S,5S)-6,6-dimethyl-N-{(2S)-3-oxo-1- |
| | | | | | [(3S)-2-oxopyrrolidin-3-yl]-4-[(1,2,5- |
| | | | | | thiadiazol-3-yl)oxy]butan-2-yl}-3-[N- |
| | | | | | (trifluoroacetyl)-L-valyl]-3- |
| | | | | | azabicyclo[3.1.0]hexane-2-carboxamide |
| 63 | 54.2 | 1 | 5550 | 2 | (1R,2S,5S)-N-{(2S)-4-(2,4-difluorophenoxy)- |
| | | | | | 3-oxo-1-[(3S)-2-oxopyrrolidin-3-yl]butan-2- |
| | | | | | yl}-6,6-dimethyl-3-[3′,3′,3′-trifluoro-N- |
| | | | | | (trifluoromethanesulfonyl)-L-isoleucyl]-3- |
| | | | | | azabicyclo[3.1.0]hexane-2-carboxamide |
| 64 | 62.2 | 2 | 5360 | 6 | (1R,2S,5S)-6,6-dimethyl-N-{(2S)-3-oxo-1- |
| | | | | | [(3S)-2-oxopyrrolidin-3-yl]-4-[(1-phenyl-1H- |
| | | | | | 1,2,4-triazol-5-yl)oxy]butan-2-yl}-3-[N- |
| | | | | | (trifluoroacetyl)-L-valyl]-3- |
| | | | | | azabicyclo[3.1.0]hexane-2-carboxamide |
| 65 | 96.7 | 2 | 133 | 2 | (1R,2S,5S)-6,6-dimethyl-3-[N-methyl-N- |
| | | | | | (propan-2-yl)-L-leucyl]-N-{(2R*)-4-{[1- |
| | | | | | methyl-3-(trifluoromethyl)-1H-pyrazol-5- |
| | | | | | yl]oxy}-3-oxo-1-[(3S)-2-oxopyrrolidin-3- |
| | | | | | yl]butan-2-yl}-3-azabicyclo[3.1.0]hexane-2- |
| | | | | | carboxamide, DIAST-1 |
| 66 | 15.7 | 3 | 669 | 2 | (5R*)-N-{(2S)-4-(2,4-difluorophenoxy)-3- |
| | | | | | oxo-1-[(3S)-2-oxopyrrolidin-3-yl]butan-2- |
| | | | | | yl}-6-[N-(methanesulfonyl)-L-valyl]-6- |
| | | | | | azaspiro[2.5]octane-5-carboxamide, |
| | | | | | DIAST-2 |
| 67 | 107 | 2 | 1750 | 6 | (1R,2S,5S)-N-{(2S)-4-[(4-ethyl-5-methyl- |
| | | | | | 1,2-oxazol-3-yl)oxy]-3-oxo-1-[(3S)-2- |
| | | | | | oxopyrrolidin-3-yl]butan-2-yl}-6,6-dimethyl- |
| | | | | | 3-[N-(trifluoroacetyl)-L-valyl]-3- |
| | | | | | azabicyclo[3.1.0]hexane-2-carboxamide |
| 68 | 129 | 2 | 97 | 4 | (1R,2S,5S)-N-{(2R)-4-(2,4- |
| | | | | | difluorophenoxy)-3-oxo-1-[(3S)-2- |
| | | | | | oxopyrrolidin-3-yl]butan-2-yl}-6,6-dimethyl- |
| | | | | | 3-[3-methyl-N-(trifluoromethanesulfonyl)-L- |
| | | | | | valyl]-3-azabicyclo[3.1.0]hexane- |
| | | | | | 2-carboxamide |
| 69 | 219 | 2 | 562 | 2 | (2R*,4S*)-N-{(2S)-4-(2,4-difluorophenoxy)- |
| | | | | | 3-oxo-1-[(3S)-2-oxopyrrolidin-3-yl]butan-2- |
| | | | | | yl}-4-methyl-1-[N-(trifluoroacetyl)-L- |
| | | | | | valyl]piperidine-2-carboxamide |
| 70 | 203 | 1 | 5440 | 2 | (1R,2S,5S)-N-{(2S)-4-(2,4-difluorophenoxy)- |
| | | | | | 3-oxo-1-[(3S)-2-oxopyrrolidin-3-yl]butan-2- |
| | | | | | yl}-6,6-dimethyl-3-[3′,3′,3′-trifluoro-N- |
| | | | | | (trifluoromethanesulfonyl)-L-isoleucyl]-3- |
| | | | | | azabicyclo[3.1.0]hexane-2-carboxamide |
| 71 | 220 | 2 | 8310 | 2 | (2R*,4S*)-N-{(2S)-4-(2,4-difluorophenoxy)- |
| | | | | | 3-oxo-1-[(3S)-2-oxopyrrolidin-3-yl]butan-2- |
| | | | | | yl}-1-[N-(methanesulfonyl)-L-valyl]-4- |
| | | | | | (trifluoromethyl)piperidine-2- |
| | | | | | carboxamide, DIAST-1 |
| 72 | 505 | 2 | 19000 | 6 | (1R,2S,5S)-6,6-dimethyl-N-{(2S)-4-[(6- |
| | | | | | methyl-1,3-benzoxazol-2-yl)oxy]-3-oxo-1- |
| | | | | | [(3S)-2-oxopyrrolidin-3-yl]butan-2-yl}-3-[N- |
| | | | | | (trifluoroacetyl)-L-valyl]-3- |
| | | | | | azabicyclo[3.1.0]hexane-2-carboxamide |
| 73 | 962 | 2 | 140 | 2 | (1R,2S,5S)-6,6-dimethyl-3-[N-methyl-N- |
| | | | | | (propan-2-yl)-L-leucyl]-N-{(2R*)-4-{[1- |
| | | | | | methyl-3-(trifluoromethyl)-1H-pyrazol-5- |
| | | | | | yl]oxy}-3-oxo-1-[(3S)-2-oxopyrrolidin-3- |
| | | | | | yl]butan-2-yl}-3-azabicyclo[3.1.0]hexane-2- |
| | | | | | carboxamide, DIAST-2 |
| 74 | 919 | 2 | 25700 | 6 | (1R,2S,5S)-6,6-dimethyl-N-{(2S)-4-{[4- |
| | | | | | methyl-5-(trifluoromethyl)-4H-1,2,4-triazol- |
| | | | | | 3-yl]oxy}-3-oxo-1-[(3S)-2-oxopyrrolidin-3- |
| | | | | | yl]butan-2-yl}-3-[N-(trifluoroacetyl)-L- |
| | | | | | valyl]-3-azabicyclo[3.1.0]hexane- |
| | | | | | 2-carboxamide |
| 75 | 2900 | 2 | >76800 | 6 | (1R,2S,5S)-N-{(2S)-4-[(8- |
| | | | | | fluoro[1,2,4]triazolo[4,3-a]pyridin-3-yl)oxy]- |
| | | | | | 3-oxo-1-[(3S)-2-oxopyrrolidin-3-yl]butan-2- |
| | | | | | yl}-6,6-dimethyl-3-[N-(trifluoroacetyl)-L- |
| | | | | | valyl]-3-azabicyclo[3.1.0]hexane- |
| | | | | | 2-carboxamide |
| 76 | 3840 | 2 | >100000 | 1 | (1R,2S,5S)-6,6-dimethyl-N-{(2S)-4-[(3- |
| | | | | | methyl-1,2,4-oxadiazol-5-yl)oxy]-3-oxo-1- |
| | | | | | [(3S)-2-oxopyrrolidin-3-yl]butan-2-yl}-3- |
| | | | | | [N-(trifluoroacetyl)-L-valyl]-3- |
| | | | | | azabicyclo[3.1.0]hexane-2-carboxamide |
| 77 | >10800 | 1 | >100000 | 1 | (2R*,4S*)-4-tert-butyl-N-{(2S)-4-methoxy- |
| | | | | | 3-oxo-1-[(3S)-2-oxopyrrolidin-3-yl]butan- |
| | | | | | 2-yl}-1-[N-(trifluoromethanesulfonyl)-L- |
| | | | | | valyl]piperidine-2-carboxamide, DIAST-2 |
| 78 | N.D. | | 7370 | 2 | (1R,2S,5S)-N-{(2S)-4-[(5-tert-butyl-4- |
| | | | | | methyl-4H-1,2,4-triazol-3-yl)oxy]-3-oxo-1- |
| | | | | | [(3S)-2-oxopyrrolidin-3-yl]butan-2-yl}-6,6- |
| | | | | | dimethyl-3-[N-(trifluoroacetyl)-L-valyl]-3- |
| | | | | | azabicyclo[3.1.0]hexane-2-carboxamide |
| 114 | 2.53 | 2 | N.D. | | (1R,2S,5S)-N-{(2S)-4-(3,4-difluorophenoxy)- |
| | | | | | 3-oxo-1-[(3S)-2-oxopyrrolidin-3-yl]butan-2- |
| | | | | | yl}-6,6-dimethyl-3-[3-methyl-N- |
| | | | | | (trifluoromethanesulfonyl)-L-valyl]-3- |
| | | | | | azabicyclo[3.1.0]hexane-2-carboxamide |

N.D. = not determined

All patents and publications described hereinabove are hereby incorporated by reference in their entirety. While the invention has been described in terms of various preferred embodiments and specific examples, the invention should be understood as not being limited by the foregoing detailed description, but as being defined by the appended claims and their equivalents.

1.-6. (canceled)

7. A compound selected from the group consisting of formulae Ia-1 through Ia-8 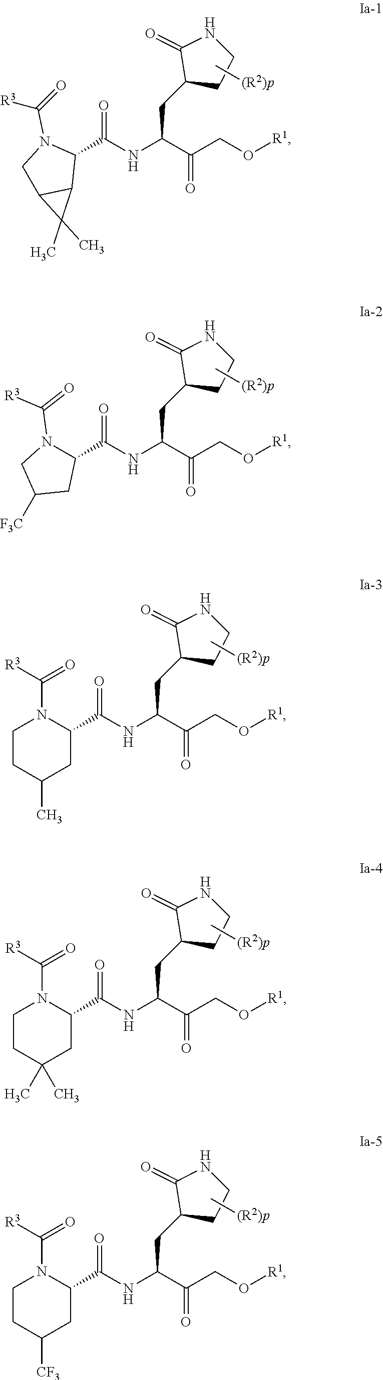 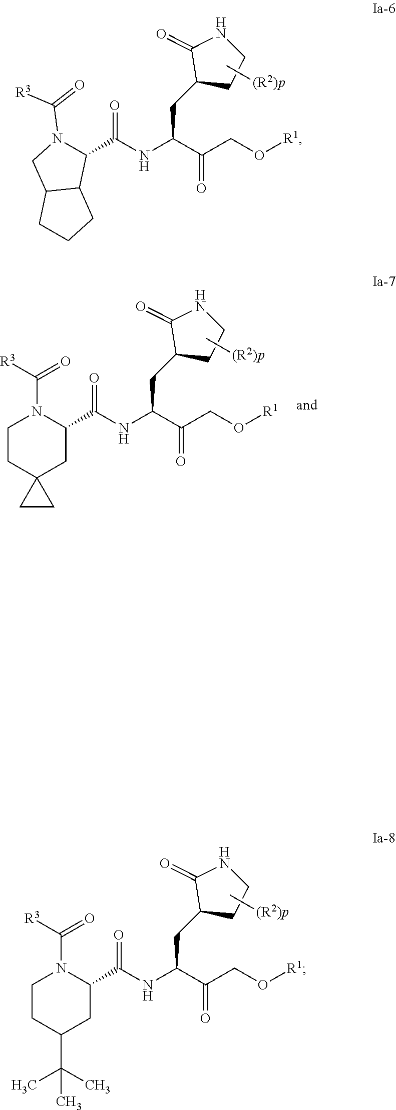
wherein
R¹ is selected from the group consisting of C₁-C₆ alkyl, C₃-C₆ cycloalkyl, (C₃-C₆ cycloalkyl)-C₁-C₆ alkyl, C₆-C₁₀ aryl, (C₆-C₁₀ aryl)-C₁-C₆ alkyl, (C₆-C₁₀ aryl)-C₂-C₆ alkenyl, (C₆-C₁₀ aryloxy)-C₁-C₆ alkyl, 5- to 10-membered heteroaryl wherein the heteroaryl moiety comprises one to five heteroatoms independently selected from N, O and S; (5- to 10-membered heteroaryl)-(C₁-C₆) alkyl wherein the heteroaryl moiety comprises one to five heteroatoms independently selected from N, O and S, (5- to 10-membered heteroaryl)-(C₂-C₆) alkenyl wherein the heteroaryl moiety comprises one to five heteroatoms independently selected from N, O and S, (5- to 10-membered heteroaryloxy)-(C₁-C₆) alkyl wherein the heteroaryl moiety comprises one to five heteroatoms independently selected from N, O and S, 4- to 12-membered heterocycloalkyl wherein said heterocycloalkyl moiety comprises one to four heteroatoms independently selected from N, O and S(O)ₙ, and (4- to 12-membered heterocycloalkyl)-C₁-C₆ alkyl wherein said heterocycloalkyl moiety comprises one to four heteroatoms independently selected from N, O and S(O)ₙ; wherein each R¹ is optionally independently substituted with one to four R^{1A}; R^{1A} at each occurrence is independently selected from the group consisting of halo, hydroxy, cyano, phenyl, benzyl, amino, (C₁-C₆ alkyl)amino optionally substituted with one to five fluoro, di(C₁-C₆ alkyl)amino optionally substituted with one to ten fluoro, C₁-C₆ alkyl optionally substituted with one to five fluoro, C1-C alkoxy optionally substituted with one to five fluoro, C₁-C₃ alkoxy-C₁-C₃ alkyl optionally substituted with one to five fluoro, C₃-C₆ cycloalkyl optionally substituted with one to three fluoro or C₁-C₃ alkyl;
R² at each occurrence is independently hydroxy or oxo;
p is 0, 1 or 2;
R³ is selected from the group consisting of C₁-C₈ alkyl, C₁-C₈ alkoxy, (C₁-C₆ alkoxy)-C₁-C₆ alkyl, C₂-C₆ alkynyl, C₂-C₆ alkynyloxy, C₃-C₁₂ cycloalkyl optionally fused with a 5- to 6-membered heteroaryl or phenyl, (C₃-C₁₂ cycloalkyl)-C₁-C₆ alkyl, C₃-C₁₂ cycloalkoxy, (C₃-C₁₂ cycloalkoxy)-C₁-C₆ alkyl, 4- to 12-membered heterocycloalkyl which is optionally fused with a 5- to 6-membered heteroaryl or phenyl and wherein said heterocycloalkyl comprises one to four heteroatoms independently selected from N, O and S(O)ₙ, (4- to 12-membered heterocycloalkyl)-C₁-C₆ alkyl wherein said heterocycloalkyl moiety comprises one to four heteroatoms independently selected from N, O and S(O)ₙ, C₆-C₁₀ aryl optionally fused with a C₄-C₆ cycloalkyl or a 4- to 7-membered heterocycloalkyl, (C₆-C₁₀ aryl)-C₁-C₆ alkyl, (C₆-C₁₀ aryloxy)-C₁-C₆ alkyl, (C₆-C₁₀ aryl)-(C₂-C₆) alkenyl, 5- to 10-membered heteroaryl comprising one to five heteroatoms independently selected from N, O and S, which is optionally fused with a C₅-C₆ cycloalkyl; (5- to 10-membered heteroaryl)-C₁-C₆ alkyl wherein the heteroaryl moiety comprises one to five heteroatoms independently selected from N, O and S; (5- to 10-membered heteroaryl)-(C₂-C₆) alkenyl wherein the heteroaryl moiety comprises one to five heteroatoms independently selected from N, O and S; (C₆-C₁₀ aryl)-(5- to 10-membered heteroaryl)- wherein the heteroaryl moiety comprises one to five heteroatoms independently selected from N, O and S, (5- to 10-membered heteroaryloxy)-C₁-C₆ alkyl wherein the heteroaryl moiety comprises one to five heteroatoms independently selected from N, O and S; (5- to 6-membered heteroaryl)-(5- to 6-membered heteroaryl)- wherein each heteroaryl moiety comprises one to four heteroatoms independently selected from N, O and S; (4- to 7-membered heterocycloalkyl)-(5- to 6-membered heteroaryl)- wherein the heterocycloalkyl moiety comprises one to three heteroatoms independently selected from N, O and S(O)ₙ and the heteroaryl moiety comprises one to four heteroatoms independently selected from N, O and S; (5- to 6-membered heteroaryl)-(4- to 7-membered heterocycloalkyl)- wherein the heterocycloalkyl moiety comprises one to three heteroatoms independently selected from N, O and S(O)ₙ and the heteroaryl moiety comprises one to four heteroatoms independently selected from N, O and S; wherein each R³ group is optionally substituted with one to five R⁴;
R⁴ at each occurrence is independently selected from the group consisting of oxo, halo, hydroxy, cyano, phenyl, benzyl, amino, (C₁-C₆ alkyl)amino optionally substituted with one to five fluoro, di(C₁-C₆ alkyl)amino optionally substituted with one to ten fluoro, C₁-C₆ alkyl optionally substituted with one to five fluoro, C₁-C₆ alkoxy optionally substituted with one to five fluoro, C₁-C₃ alkoxy-C₁-C₃ alkyl optionally substituted with one to five fluoro, C₃-C₆ cycloalkyl optionally substituted with one to three fluoro or C₁-C₃ alkyl, C₁-C₆ alkyl-C(O)NH— optionally substituted with one to five fluoro or with one R⁵, C₁-C₆ alkyl-OC(O)NH— optionally substituted with one to five fluoro or with one R⁵, C₁-C₆ alkyl-NHC(O)NH— optionally substituted with one to five fluoro or with one R⁵, C₁-C₆ alkyl-S(O)₂NH— optionally substituted with one to five fluoro or with one R⁵, C₁-C₆ alkyl-C(O)— optionally substituted with one to five fluoro or with one R⁵, C₁-C₆ alkyl-S(O)ₙ— optionally substituted with one to five fluoro or with one R⁵;
R⁵ is selected from phenyl, phenoxy, C₃-C₆ cycloalkyl, C₃-C₆ cycloalkoxy, 4- to 7-membered heterocycloalkyl- wherein the heterocycloalkyl moiety comprises one to three heteroatoms independently selected from N, O and S(O)ₙ and 5- to 6-membered heteroaryl- wherein the heteroaryl moiety comprises one to four heteroatoms independently selected from N, O and S; wherein each R⁵ is optionally independently substituted with one to three halo, C₁-C₃ alkyl and C₁-C₃ alkoxy; and
n at each occurrence is independently selected from 0, 1 and 2;
or a pharmaceutically acceptable salt thereof.

8. The compound of claim 7 selected from the group consisting of 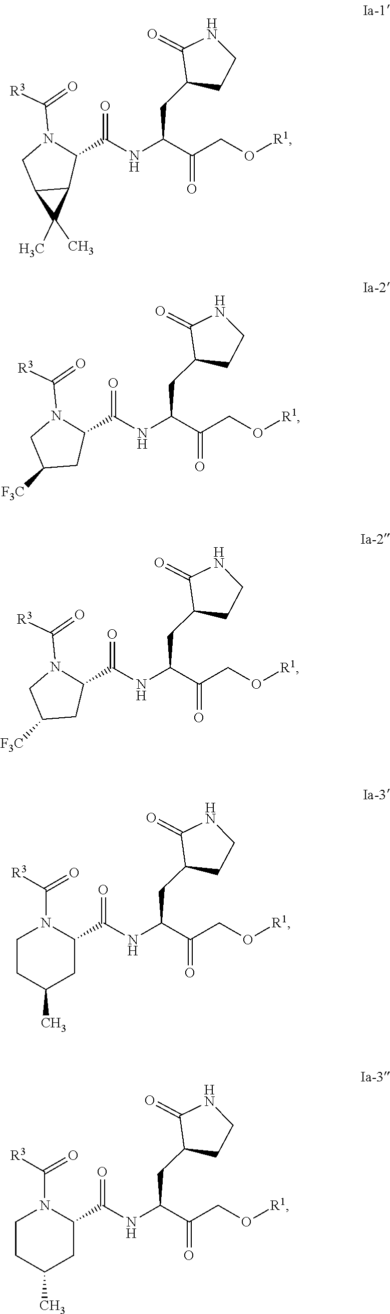 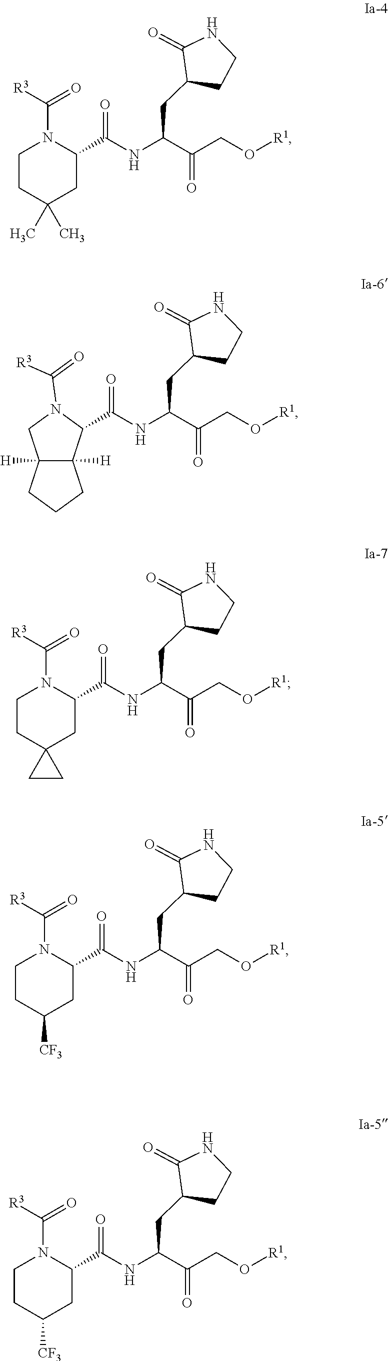 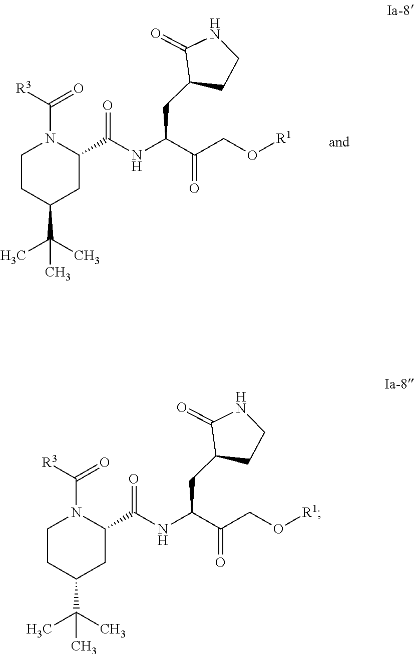
or a pharmaceutically acceptable salt thereof.

9. The compound of claim 8 wherein R³ is substituted with one R⁴ selected from the group consisting of C₁-C₆ alkyl-C(O)NH— optionally substituted with one to five fluoro or with one R⁵, C₁-C₆ alkyl-OC(O)NH— optionally substituted with one to five fluoro or with one R⁵, C₁-C₆ alkyl-NHC(O)NH— optionally substituted with one to five fluoro or with one R⁵, C₁-C₆alkyl-S(O)₂NH— optionally substituted with one to five fluoro or with one R⁵, C₁-C₆ alkyl-C(O)— optionally substituted with one to five fluoro or with one R⁵, C₁-C₆ alkyl-S(O)ₙ— optionally substituted with one to five fluoro or with one R⁵; and the R³ is optionally substituted with one to three R⁴; or a pharmaceutically acceptable salt thereof.

10. The compound of claim 9 wherein R³ is selected from the group consisting of C₁-C₆ alkyl, (C₁-C₆ alkoxy)-C₁-C₆ alkyl, C₃-C₇ cycloalkyl and (C₃-C₇ cycloalkyl)-C₁-C₆ alkyl, each of which is substituted with one R⁴ selected from the group consisting of CF₃S(O)₂NH—, CH₃S(O)₂NH—, CF₃C(O)NH—, CH₃C(O)NH— and CH₃OC(O)NH—, and is optionally substituted with one to three R⁴; or a pharmaceutically acceptable salt thereof.

11. The compound of claim 10 wherein R³ is selected from the group consisting of 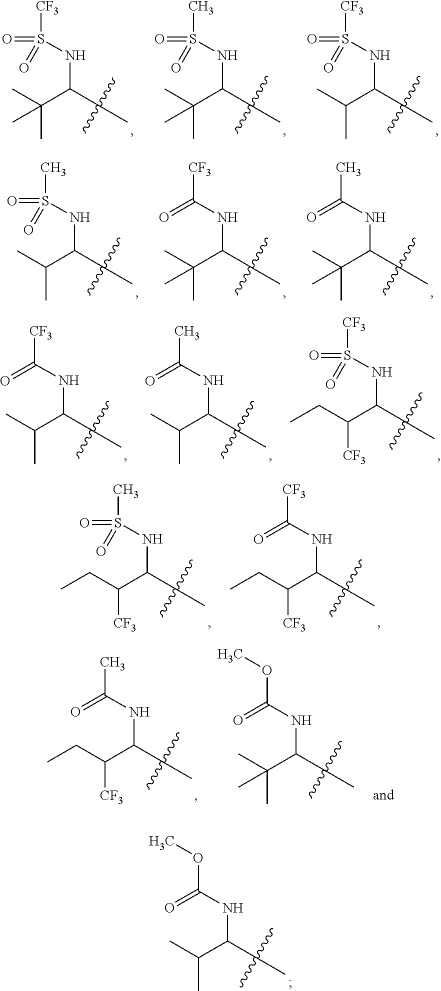
or a pharmaceutically acceptable salt thereof.

12. The compound of claim 7 wherein R¹ is phenyl which is optionally substituted with one to three R^{1A}; or a pharmaceutically acceptable salt thereof.

13. The compound of claim 7 wherein R¹ is benzyl which is optionally substituted with one to three R^{1A}; or a pharmaceutically acceptable salt thereof.

14. The compound of claim 7 wherein R¹ is a 5- to 10-membered heteroaryl wherein the heteroaryl moiety comprises one to four heteroatoms independently selected from N, O and S, and the heteroaryl is optionally substituted with one to three R^{1A}; or a pharmaceutically acceptable salt thereof.

15. The compound of claim 14 wherein R¹ is a 5- to 10-membered heteroaryl selected from the group consisting of pyrrolyl, furanyl, thienyl, imidazolyl, pyrazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl, indolyl, benzoxazolyl, benzimidazolyl, benzothiazolyl, pyridinopyrrolyl, quinolinyl, quinoxalinyl, benzotriazolyl, imidazo[1,2-a]pyridinyl, imidazo[2,1-b][1,3]thiazolyl, 4H-furo[3,2-b]pyrrolyl, 4H-thieno[3,2-b]pyrrolyl, [1,2,4]triazolo[1,5-a]pyrimidinyl, [1,2,3]triazolo[1,5-a]pyridinyl and naphthyridinyl; each of which is optionally substituted with one to three R^{1A}; or a pharmaceutically acceptable salt thereof.

16. The compound of claim 7 wherein R¹ is C₁-C₆ alkyl which is optionally substituted with one to three R^{1A}; or a pharmaceutically acceptable salt thereof.

17. The compound of claim 16 wherein R¹ is selected from methyl, ethyl, isopropyl, 2,2,2-trifluoroethyl and 1,1,1,3,3,3-hexafluoropropan-2-yl; or a pharmaceutically acceptable salt thereof.

18. The compound of claim 7 wherein R^{1A} at each occurrence is independently selected from the group consisting of chloro, fluoro, cyano, methyl, difluoromethyl, trifluoromethyl, ethyl, propyl, isopropyl, butyl, tert-butyl, methoxy and trifluoromethoxy; or a pharmaceutically acceptable salt thereof.

19. A compound selected from the group consisting of
(1R,2S,5S)-N-{(2S)-4-[(4-methoxyphenyl)methoxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(2R*,4S*)-4-tert-butyl-N-{(2S)-4-methoxy-3-oxo-1-[(3S)-2-oxopyrrolidin-3-yl]butan-2-yl}-1-[N-(trifluoromethanesulfonyl)-L-valyl]piperidine-2-carboxamide, DIAST-1;
methyl {(2S)-1-[(1R,2S,5S)-2-({(2S)-4-(3-chlorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-6,6-dimethyl-2-({(2S)-3-oxo-1-[(3S)-2-oxopyrrolidin-3-yl]-4-[3-(trifluoromethyl)phenoxy]butan-2-yl}carbamoyl)-3-azabicyclo[3.1.0]hexan-3-yl]-3,3-dimethyl-1-oxobutan-2-yl}carbamate;
(1R,2S,5S)-6,6-dimethyl-N-{(2S)-4-[(6-methylpyridin-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-[(6-methylpyridin-3-yl)oxy]-3-oxo-1-[(3S)-2-oxopyrrolidin-3-yl]butan-2-yl}-3-[3-methyl-N-(trifluoroacetyl)-L-valyl]-3-azabicyclo[3.1.0]hexane-2-carboxamide;
(1R,2S,5S)-6,6-dimethyl-3-[3-methyl-N-(trifluoroacetyl)-L-valyl]-N-{(2S)-3-oxo-1-[(3S)-2-oxopyrrolidin-3-yl]-4-phenoxybutan-2-yl}-3-azabicyclo[3.1.0]hexane-2-carboxamide
methyl {(2S)-1-[(1R,2S,5S)-2-({(2S)-4-(4-fluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-2-({(2S)-4-(2-fluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-6,6-dimethyl-N-{(2S)-3-oxo-1-[(3S)-2-oxopyrrolidin-3-yl]-4-phenoxybutan-2-yl}-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-3-[N-(methanesulfonyl)-3-methyl-L-valyl]-6,6-dimethyl-3-azabicyclo[3.1.0]hexane-2-carboxamide;
methyl {(2S)-1-[(1R,2S,5S)-2-({(2S)-4-(2,4-difluorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
methyl {(2S)-1-[(1R,2S,5S)-2-({(2S)-4-(3-chlorophenoxy)-3-oxo-1-[(3S)-2-oxopyrrolidin-3-yl]butan-2-yl}carbamoyl)-6,6-dimethyl-3-azabicyclo[3.1.0]hexan-3-yl]-3-methyl-1-oxobutan-2-yl}carbamate;
(1R,2S,5S)-3-[N-(methanesulfonyl)-3-methyl-L-valyl]-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-[(3-ethyl-1-methyl-1H-pyrazol-5-yl)oxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-[(1-ethyl-3-methyl-1H-pyrazol-5-yl)oxy]-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2R)-4-(4-fluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
methyl {(2S)-1-[(1R,2S,5S)-6,6-dimethyl-2-({(2S)-3-oxo-1-[(3S)-2-oxopyrrolidin-3-yl]-4-phenoxybutan-2-yl}carbamoyl)-3-azabicyclo[3.1.0]hexan-3-yl]-3,3-dimethyl-1-oxobutan-2-yl}carbamate;
(1R,2S,5S)-N-{(2S)-4-[(6-fluoropyridin-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoroacetyl)-L-isoleucyl]-3-azabicyclo[3.1.0]hexane-2-carboxamide;
methyl {(2S)-1-[(1R,2S,5S)-2-({(2S)-4-[(1-ethyl-3-methyl-1H-pyrazol-5-yl)oxy]-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-6,6-dimethyl-3-[3-methyl-N-(trifluoroacetyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-(2,5-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2RS)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
methyl {(2S)-1-[(1R,2S,5S)-6,6-dimethyl-2-({(2S)-4-{[1-methyl-3-(trifluoromethyl)-1H-pyrazol-5-yl]oxy}-3-oxo-1-[(3S)-2-oxopyrrolidin-3-yl]butan-2-yl}carbamoyl)-3-azabicyclo[3.1.0]hexan-3-yl]-3-methyl-1-oxobutan-2-yl}carbamate;
methyl {(2S)-1-[(1R,2S,5S)-2-({(2S)-4-[(3-ethyl-1-methyl-1H-pyrazol-5-yl)oxy]-3-oxo-1-[(3S)-2-oxopyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoroacetyl)-L-valyl]-N-{(2S)-3-oxo-1-[(3S)-2-oxopyrrolidin-3-yl]-4-[3-(trifluoromethyl)phenoxy]butan-2-yl}-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[N-(trifluoromethanesulfonyl)-L-valyl]-3-azabicyclo[3.1.0]hexane-2-carboxamide;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[N-(trifluoromethanesulfonyl)-L-valyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4-(trifluoromethyl)piperidine-2-carboxamide, DIAST-2;
(1R,2S,5S)-N-{(2S)-4-(2,4-difluorophenoxy)-1-[(3R)-2,5-dioxopyrrolidin-3-yl]-3-oxobutan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[3-(trifluoromethyl)phenoxy]butan-2-yl}-3-azabicyclo[3.1.0]hexane-2-carboxamide;
(1R,2S,5S)-N-{(2S)-4-[(1-ethyl-3-methyl-1H-pyrazol-5-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-1-[(3R)-2,5-dioxopyrrolidin-3-yl]-3-oxobutan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-[(3-ethyl-1-methyl-1H-pyrazol-5-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(4-methyl-1,2,5-thiadiazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[3-(trifluoromethyl)phenoxy]butan-2-yl}-3-[N-(trifluoromethanesulfonyl)-L-valyl]-3-azabicyclo[3.1.0]hexane-2-carboxamide;
(1R,2S,5S)-N-{(2S)-4-(2-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(3,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-[N-(trifluoromethanesulfonyl)-L-valyl]-3-azabicyclo[3.1.0]hexane-2-carboxamide;
(1R,2S,5S)-N-{(2S)-4-(2,4-difluorophenoxy)-1-[(3R)-2,5-dioxopyrrolidin-3-yl]-3-oxo butan-2-yl}-6,6-dimethyl-3-[N-(trifluoromethanesulfonyl)-L-valyl]-3-azabicyclo[3.1.0]hexane-2-carboxamide;
(1R,2S,5S)-N-{(2S)-4-(3-chlorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(6-methyl-1,2-benzoxazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(4-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,5-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-(2,4-difluorophenoxy)-1-[(3R)-5-hydroxy-2-oxopyrrolidin-3-yl]-3-oxobutan-2-yl}-6,6-dimethyl-3-[N-(trifluoromethanesulfonyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-1,2,4-triazol-5-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[3-(propan-2-yl)phenoxy]butan-2-yl}-3-[N-(trifluoroacetyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoroacetyl)-L-isoleucyl]-3-azabicyclo[3.1.0]hexane-2-carboxamide;
(1R,2S,5S)-N-{(2S)-4-(3,5-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(5R*)-N-{(2S)-4-(2,4-difluorophenoxy)-3-oxo-1-[(3S)-2-oxopyrrolidin-3-yl]butan-2-yl}-6-[N-(methanesulfonyl)-L-valyl]-6-azaspiro[2.5]octane-5-carboxamide, DIAST-1;
(2R*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4,4-dimethylpiperidine-2-carboxamide;
(1R,2S,5S)-6,6-dimethyl-N-{(2S)-4-[(5-methyl-1,2-oxazol-3-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[(1,2,5-thiadiazol-3-yl)oxy]butan-2-yl}-3-[N-(trifluoroacetyl)-L-valyl]-3-azabicyclo[3.1.0]hexane-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoromethanesulfonyl)-L-isoleucyl]-3-azabicyclo[3.1.0]hexane-2-carboxamide;
(1R,2S,5S)-6,6-dimethyl-N-{(2S)-3-oxo-1-[(3S)-2-oxopyrrolidin-3-yl]-4-[(1-phenyl-1H-1,2,4-triazol-5-yl)oxy]butan-2-yl}-3-[N-(trifluoroacetyl)-L-valyl]-3-azabicyclo[3.1.0]hexane-2-carboxamide;
(1R,2S,5S)-6,6-dimethyl-3-[N-methyl-N-(propan-2-yl)-L-leucyl]-N-{(2R*)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide, DIAST-1;
(5R*)-N-{(2S)-4-(2,4-difluorophenoxy)-3-oxo-1-[(3S)-2-oxopyrrolidin-3-yl]butan-2-yl}-6-[N-(methanesulfonyl)-L-valyl]-6-azaspiro[2.5]octane-5-carboxamide, DIAST-2;
(1R,2S,5S)-N-{(2S)-4-[(4-ethyl-5-methyl-1,2-oxazol-3-yl)oxy]-3-oxo-1-[(3S)-2-oxo pyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2R)-4-(2,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2R)-4-(2,4-difluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-4-methyl-1-[N-(trifluoroacetyl)-L-valyl]piperidine-2-carboxamide;
(1R,2S,5S)-N-{(2S)-4-(2,4-difluorophenoxy)-3-oxo-1-[(3S)-2-oxopyrrolidin-3-yl]butan-2-yl}-6,6-dimethyl-3-[3′,3′,3′-trifluoro-N-(trifluoromethanesulfonyl)-L-isoleucyl]-3-azabicyclo[3.1.0]hexane-2-carboxamide;
(2R*,4S*)-N-{(2S)-4-(2,4-difluorophenoxy)-3-oxo-1-[(3S)-2-oxopyrrolidin-3-yl]butan-2-yl}-1-[N-(methanesulfonyl)-L-valyl]-4-(trifluoromethyl)piperidine-2-carboxamide, DIAST-1;
(1R,2S,5S)-6,6-dimethyl-N-{(2S)-4-[(6-methyl-1,3-benzoxazol-2-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-3-[N-methyl-N-(propan-2-yl)-L-eucyl]-N-{(2R*)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide, DIAST-2;
(1R,2S,5S)-6,6-dimethyl-N-{(2S)-4-{[4-methyl-5-(trifluoromethyl)-4H-1,2,4-triazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-[(8-fluoro[1,2,4]triazolo[4,3-a]pyridin-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-[(3-methyl-1,2,4-oxadiazol-5-yl)oxy]-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(2R*,4S*)-4-tert-butyl-N-{(2S)-4-methoxy-3-oxo-1-[(3S)-2-oxopyrrolidin-3-yl]butan-2-yl}-1-[N-(trifluoromethanesulfonyl)-L-valyl]piperidine-2-carboxamide, DIAST-2;
(1R,2S,5S)-N-{(2S)-4-[(5-tert-butyl-4-methyl-4H-1,2,4-triazol-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[4-methyl-5-(propan-2-yl)-4H-1,2,4-triazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-6,6-dimethyl-N-{(2S)-4-(2-methylphenoxy)-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(3-cyanophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-[(5-tert-butyl-4-methyl-1,2-oxazol-3-yl)oxy]-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-6,6-dimethyl-N-{(2S)-4-{[1-methyl-5-(trifluoromethyl)-1H-pyrazol-3-yl]oxy}-3-oxo-1-[(3S)-2-oxopyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-[(5-fluoro-1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-methoxy-3-oxo-1-[(3S)-2-oxopyrrolidin-3-yl]butan-2-yl}-6,6-dimethyl-3-[3-methyl-N-(trifluoroacetyl)-L-valyl]-3-azabicyclo[3.1.0]hexane-2-carboxamide;
(1R,2S,5S)-N-{(2S)-4-methoxy-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
methyl {(2S)-1-[(1R,2S,5S)-6,6-dimethyl-2-({(2S)-4-{[1-methyl-3-(trifluoromethyl)-1H-pyrazol-5-yl]oxy}-3-oxo-1-[(3S)-2-oxopyrrolidin-3-yl]butan-2-yl}carbamoyl)-3-azabicyclo[3.1.0]hexan-3-yl]-3,3-dimethyl-1-oxobutan-2-yl}carbamate;
methyl {(2S)-1-[(1R,2S,5S)-6,6-dimethyl-2-({(2S)-4-[(6-methylpyridin-3-yl)oxy]-3-oxo-1-[(3S)-2-oxopyrrolidin-3-yl]butan-2-yl}carbamoyl)-3-azabicyclo[3.1.0]hexan-3-yl]-3,3-dimethyl-1-oxobutan-2-yl}carbamate;
(1R,2S,5S)-N-{(2S)-4-(2-fluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
methyl {(2S)-1-[(1R,2S,5S)-2-({(2S)-4-(2,5-difluorophenoxy)-3-oxo-1-[(3S)-2-oxo pyrrolidin-3-yl]butan-2-yl}carbamoyl)-6,6-dimethyl-3-azabicyclo[3.1.0]hexan-3-yl]-3,3-dimethyl-1-oxobutan-2-yl}carbamate;
(1R,2S,5S)-N-{(2S)-4-methoxy-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-N-{(2S)-4-[(2-methylpyridin-4-yl)oxy]-3-oxo-1-[(3S)-2-oxo pyrrolidin-3-yl]butan-2-yl}-3-[N-(trifluoroacetyl)-L-valyl]-3-azabicyclo[3.1.0]hexane-2-carboxamide;
(1R,2S,5S)-N-{(2S)-4-(5-chloro-2-fluorophenoxy)-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(1R,2S,5S)-N-{(2S)-4-{[5-(difluoromethyl)-1-methyl-1H-pyrazol-3-yl]oxy}-3-oxo-1-[(3S)-2-oxopyrrolidin-3-yl]butan-2-yl}-6,6-dimethyl-3-[N-(trifluoroacetyl)-L-valyl]-3-azabicyclo[3.1.0]hexane-2-carboxamide;
(2S,4R)-4-tert-butyl-N-{(2S)-4-methoxy-3-oxo-1-[(3S)-2-oxopyrrolidin-3-yl]butan-2-yl}-1-[3-methyl-N-(trifluoromethanesulfonyl)-L-valyl]piperidine-2-carboxamide;
3-methyl-N-(trifluoromethanesulfonyl)-L-valyl-(4R)-N-{(2S)-4-(2,4-difluorophenoxy)-3-oxo-1-[(3S)-2-oxopyrrolidin-3-yl]butan-2-yl}-4-(trifluoromethyl)-L-prolinamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[3-(trifluoromethoxy)phenoxy]butan-2-yl}-3-azabicyclo[3.1.0]hexane-2-carboxamide;
(1R,2S,5S)-N-{(2S)-4-(4-cyanophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[(1,2-thiazol-3-yl)oxy]butan-2-yl}-3-azabicyclo[3.1.0]hexane-2-carboxamide;
(1R,2S,5S)-N-{(2S)-4-[(1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-N-{(2S)-4-[(2-methylpyridin-3-yl)oxy]-3-oxo-1-[(3S)-2-oxopyrrolidin-3-yl]butan-2-yl}-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-phenoxybutan-2-yl}-3-azabicyclo[3.1.0]hexane-2-carboxamide;
(1R,2S,5S)-N-{(2S)-4-[(5-chloro-1,3-benzoxazol-2-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-N-{(2S)-4-(3-ethylphenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2S)-3-oxo-1-[(3S)-2-oxopyrrolidin-3-yl]-4-[(1,3-thiazol-2-yl)oxy]butan-2-yl}-3-azabicyclo[3.1.0]hexane-2-carboxamide;
(1R,2S,5S)-N-{(2S)-4-[(1-ethyl-1H-pyrazol-3-yl)oxy]-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
3-methyl-N-(trifluoromethanesulfonyl)-L-valyl-(4R)-N-{(2S)-4-(3-chlorophenoxy)-3-oxo-1-[(3S)-2-oxopyrrolidin-3-yl]butan-2-yl}-4-(trifluoromethyl)-L-prolinamide;
3-methyl-N-(trifluoromethanesulfonyl)-L-valyl-(4R)-N-{(2S)-4-{[1-methyl-3-(trifluoromethyl)-1H-pyrazol-5-yl]oxy}-3-oxo-1-[(3S)-2-oxopyrrolidin-3-yl]butan-2-yl}-4-(trifluoromethyl)-L-prolinamide;
(1R,2S,5S)-N-{(2S)-4-(3,4-difluorophenoxy)-3-oxo-1-[(3S)-2-oxopyrrolidin-3-yl]butan-2-yl}-6,6-dimethyl-3-[3-methyl-N-(trifluoromethanesulfonyl)-L-valyl]-3-azabicyclo[3.1.0]hexane-2-carboxamide;
(1R,2S,5S)-6,6-dimethyl-3-[3-methyl-N-(trifluoromethanesulfonyl)-L-valyl]-N-{(2R)-4-{[1-methyl-3-(trifluoromethyl)-1H-pyrazol-5-yl]oxy}-3-oxo-1-[(3S)-2-oxopyrrolidin-3-yl]butan-2-yl}-3-azabicyclo[3.1.0]hexane-2-carboxamide;
(2S,4R)-4-tert-butyl-1-{(2S)-2-cyclopentyl-2-[(trifluoromethanesulfonyl)amino]acetyl}-N-{(2S)-4-methoxy-3-oxo-1-[(3S)-2-oxopyrrolidin-3-yl]butan-2-yl}piperidine-2-carboxamide;
(1S,3aR,6aS)-N-{(2S)-4-methoxy-3-oxo-1-[(3S)-2-oxopyrrolidin-3-yl]butan-2-yl}-2-{(2S)-3-methyl-2-[(trifluoromethanesulfonyl)amino]butanoyl}octahydrocyclopenta[c]pyrrole-1-carboxamide; and
(1S,3aR,6aS)-2-{(2S)-3,3-dimethyl-2-[(trifluoromethanesulfonyl)amino]butanoyl}-N-{(2S)-4-methoxy-3-oxo-1-[(3S)-2-oxopyrrolidin-3-yl]butan-2-yl}octahydrocyclopenta[c]pyrrole-1-carboxamide;
or a pharmaceutically acceptable thereof.

20. A pharmaceutical composition comprising a therapeutically effective amount of a compound according to claim 7; or a pharmaceutically acceptable salt thereof and a pharmaceutically acceptable carrier.

21. A method of treating a coronavirus infection in a patient, the method comprising administering a therapeutically effective amount of a compound according to claim 7; or a pharmaceutically acceptable salt thereof.

22. The method of claim 21 wherein the coronavirus infection is COVID-19.

23. The method of claim 21 wherein the compound; or a pharmaceutically acceptable salt thereof is administered orally.

24. The method of claim 21 further comprising administration of one or more additional therapeutic agents.

25. The method of claim 24 wherein the one or more additional therapeutic agent is selected from the group consisting of remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, ABX464, dexamethasone, hydrocortisone, convalescent plasma, gelsolin (Rhu-p65N), regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, casirivimab/imdevimab (REGN-Cov2), MK-7110 (CD24Fc/SACCOVID), heparin, apixaban, tocilizumab (Actemra), sarilumab (Kevzara), apilimod dimesylate, DNL758, DC402234, PB1046, dapaglifozin, abivertinib, ATR-002, bemcentinib, acalabrutinib, baricitinib, tofacitinib, losmapimod, famotidine, niclosamide and diminazene.

26. The method of claim 25 wherein the one or more additional therapeutic agent is selected from the group consisting of remdesivir, dexamethasone, bamlanivimab, casirivimab/imdevimab, tofacitinib and baricitinib.

27.-32. (canceled)

33. A pharmaceutical composition comprising a therapeutically effective amount of a compound according to claim 19; or a pharmaceutically acceptable salt thereof and a pharmaceutically acceptable carrier.

34. A method of treating a coronavirus infection in a patient, the method comprising administering a therapeutically effective amount of a compound according to claim 19; or a pharmaceutically acceptable salt thereof.

35. The method of claim 34 wherein the coronavirus infection is COVID-19.
